(19) United States
(12) Patent Application Publication
(10) Pub. No.: US 2017/0151149 A1
(43) Pub. Date: Jun. 1, 2017

(54) TOPICAL SKIN COMPOSITIONS HAVING PROTEINS AND METHODS OF USE

(71) Applicant: Dermaforce Holdings, LLC, Phoenix, TX (US)

(72) Inventors: Jonah SHACKNAI, Phoenix, AZ (US); Mitchell S. WORTZMAN, Phoenix, AZ (US); David H. McDANIEL, Phoenix, AZ (US); Stephen NEWHART, Phoenix, AZ (US)

(21) Appl. No.: 15/365,045

(22) Filed: Nov. 30, 2016

Related U.S. Application Data
(60) Provisional application No. 62/261,285, filed on Nov. 30, 2015.

(51) Int. Cl. A61K 8/64; A61K 9/00; A61Q 19/00; A61Q 19/02; A61Q 17/04; A61K 35/33; A61Q 19/08

(52) U.S. Cl. CPC A61K 8/64; A61K 35/33; A61K 9/0014; A61Q 19/08; A61Q 19/02; A61Q 17/04; A61Q 19/00; A61K 2800/74

(57) ABSTRACT

There is provided a topical skin composition directed to one or more desired benefits. The composition includes one or more proteins selected based on the desired skin benefit to be derived by application of the topical skin composition. In one embodiment, each protein is from one donor group having two or more individuals. In another embodiment, the protein is from two or more donor group.

### CROSS REFERENCE TO RELATED APPLICATIONS

This application claims the benefit of U.S. Provisional Application Ser. No. 62/261,285, filed Nov. 30, 2015, the contents of which are incorporated by reference herein.

### BACKGROUND OF THE DISCLOSURE

1. Field of the Disclosure

The present disclosure relates to a determination of protein production levels in cultured fibroblast from human dermal tissue samples. The present disclosure also relates to a determination of protein production levels in cultured fibroblast supernatant from human dermal tissue samples from different ethnic populations through protein microarray performance. The present disclosure further relates to analyzing protein microarray performance to understand protein production levels that are present and unique in each ethnic group. Proteins can be blended together in a product that imparts one or more desired characteristics to the skin of a recipient who is not a source of the mixture and any individual fibroblasts, hereinafter a non-autologous product.

2. Description of the Related Art

The growth of eukaryotic cells is modulated by various influences, of which growth factors are among the most important. Factors which inhibit growth also exist. Growth factors have mitogenic effects on a range of cells. These mitogens stimulate the growth and activation of various cell types, including fibroblasts (which produce collagen and elastin precursors, and ground substance) and epithelial cells (e.g. skin cells or keratinocytes).

One primary function of a fibroblast is to maintain the structural integrity of connective tissues by continuously secreting precursors of the extracellular matrix. A fibroblast secretes precursors of all components of the extracellular matrix, primarily the ground substance and a variety of fibers or structural proteins. The fibroblasts also secrete small molecular weight diffusible factors that influence and coordinate the function and product of neighboring cells to enhance tissue response. The composition of the extracellular matrix significantly determines the physical properties of connective tissues.

Known in the art are methods of treatment using autologous fibroblasts (i.e. fibroblast obtained from a donor who will also be the recipient of cultured fibroblasts). Among the known uses of such fibroblasts are a method of promoting healing of wounds, such as an epithelial wound or fistula, by administering cultured fibroblasts; a method of corrective surgery by the augmentation of tissue sub-adjacent to a vocal cord defect; and a method of treatment of vocal fold scarring and repair of skin and soft tissue defects.

Also known in the art are dosage units consisting of autologous fibroblasts grown for an individual who is also the donor. Further, there are known methods of growing fibroblasts for use in autologous applications.

Applicants of the present application have filed an earlier patent application directed to a non-autologous product that is a homogeneous or heterogeneous mixture of two or more fibroblasts cultures, extracts derived therefrom, and/or diffusible elements recovered from the culture media from the same sex. It was believed that each mixture of homogeneous and/or heterogeneous fibroblasts cultures or extracts therefrom, from the same sex, can have “weighted” factors based on the characteristics desired to be obtained by the mixture and, thus, the resultant product could be tailored for certain objectives. The resultant product based on weighted factors and/or tailoring could be a product for one ethnic group, or could combine factors from two or more ethnic groups for a resultant product that would have product benefits that are universal. The mechanism to achieve these products were based on a belief that certain proteins or enzymes are more prevalent or unique or inherent from people of certain ethnic groups and thus a blending of factors in an ethnic group or a blend of factors from two or more ethnic groups would achieve the optimum product. Heretofore, there was a need to better understand what proteins and levels thereof are clearly different in each ethic group and, if so, how different.

### SUMMARY OF THE DISCLOSURE

The present disclosure provides a determination of protein production levels in cultured fibroblast supernatant from human dermal tissue samples from different ethnic populations using a protein microarray.

The present disclosure also provides for protein microarray analysis to understand differences in protein production levels in an ethnic group and/or between or amongst two or more ethnic groups.

The present disclosure further provides analysis of relative measurements of protein production levels, interactions, and/or functions, to determine what proteins or cells or factors, can be blended together, from a single ethnic group, or two or more ethnic groups, to formulate a product that imparts desired characteristics or benefits to the skin of a recipient who is not a source of the mixture or any individual fibroblasts, so that the product is defined as a non-autologous product.

The present disclosure still further provides that based on the knowledge of the measurement of the relationship of protein levels, interactions, and/or functions in an ethnic group, as well as between or amongst two or more ethnic groups, methods of optimizing the potency or potential of the proteins in a mixture to impart the desired characteristics to the recipient's skin can be achieved.

The present disclosure also provides that such measured proteins can lead to the derivation of a homogeneous and/or heterogeneous mixture that is based on the characteristics desired to be obtained to produce the desired characteristics in a resultant product.

The present disclosure further provides that different resultant products can be made with each resultant product directed to enhance, modulate, or treat one or more desired characteristics of the user of the composition of the present disclosure.

The present disclosure also provides that Biological Significance demonstrated a majority of proteins met the criteria in at least one ethnic group.

The present disclosure further provides that there are uniquely altered proteins, which is Biologically Significant proteins.

The present disclosure shows that there are average production level changes relative to control in all three groups.

The present disclosure provides a topical composition to treat a targeted skin disorder based on a desired benefit or treatment.

The present disclosure also shows that according to statistical significance, the Asian skin demonstrated the largest number or amount of protein production levels altered/dysregulated/varied relative to African skin, and the second largest amount of protein production levels altered/dysregulated/varied relative to Caucasian skin, while the least amount of protein production levels altered/dysregulated/varied was African skin relative to Caucasian skin.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is a concept map describing various embodiments of the present disclosure.

FIG. 2 shows an exemplary process for producing the embodiments of the present disclosure.

FIG. 3 shows an overall protein network expressed for the African and Asian groups.

FIG. 4 shows an overall protein network expressed for the Caucasian and Asian groups.

FIG. 5 shows an overall protein network expressed for the Caucasian and African groups.

FIG. 6 is a pie chart of a Caucasian to African interpretation of top-50 biological processes.

FIG. 7 is a pie chart of an African to Asian interpretation of top-50 biological processes.

FIG. 8 is a pie chart of a Caucasian to Asian interpretation of top-50 biological processes.

FIG. 9 shows an overall protein network expressed for the African to Asian groups relating to a wound healing process.

FIG. 10 shows an overall protein network expressed for the Caucasian to Asian groups relating to an immune system process.

FIG. 11 shows an overall protein network expressed for the Caucasian to African groups relating to an immune system process.

FIG. 12 shows several Venn Diagrams comparing the differential gene expression among the Caucasian, African, and Asian ethnic groups.

### DESCRIPTION OF THE PREFERRED EMBODIMENTS

As used herein, the term “homogeneous” means the use of fibroblast cultures obtained from donors (of the same-sex) who constitute a group whose bloodlines are 80% or greater of a single race or ethnicity, preferably 90% or greater, more preferably 95% or greater, and most preferably essentially 100%. Thus, a fibroblast culture obtained from a homogeneous set of donors (of the same-sex), such as a group of: Asian women, African women, Caucasian women, Asian men, African men, or Caucasian men, are from a single source or ethnic group or ethnic subgroup. Mixed-sex combinations are also envisioned.

The term “heterogeneous” as used herein means the use of non-ethnic homogeneity fibroblasts, such as fibroblasts obtained from same-sex, but a combination of two from different groups or sources, such as, for example, a group of: African women and Asian women, African women and Caucasian women, or Asian women and Caucasian women, Asian women and Caucasian women and African women, African men and Asian men, African men and Caucasian men, Asian men and Caucasian men, or Asian men and Caucasian men and African men. Although discussed with respect to three ethnic groups, the present disclosure provides for many more ethnic groups and subgroups so that the heterogenous mixtures can be a blend or combination of four or more ethnic groups or subgroups. Mixed-sex combinations are also envisioned.

Biological Significance or Biologically Significant means greater than a twofold increase/decrease relative to a comparator sample or a Control.

Unique is defined herein as Biologically Significant in only one group relative to a comparator sample or a Control.

A donor, as used herein, is an individual from whom cells are obtained for culturing to derive the product of the present disclosure. The donors are either male or female, or both. A donor is selected for having a desired benefit or characteristic or combination of skin characteristics.

A Caucasian donor is defined as an individual of northern European descent, whose grandparents and great-grand parents are known to have genetic roots from the area. It is envisioned that a Caucasian donor group can be further subdivided into, for example, Latin European, Hispanic European, Anglo-Saxon European, and Slavic.

An African donor is defined as an individual of sub-Saharan descent, whose grandparents and great-grandparents are known to have genetic roots from the area. It is envisioned that an African donor group can be further subdivided, including, but not limited to, West African, and East African.

An Asian donor is defined as an individual of Asian descent, or subgroups thereof including, for example: northern and southern Asia. Moreover, Asian donor group can be subdivided into; China, Japan, and Korea subgroups. An Asian donor is one whose grandparents and great-grandparents are known to have genetic roots from the area.

A donor is preferably screened for disease. Also, premature aging due to environmental conditions, such as free radical generating, namely sun and higher neoplasms, as well as premature aging due to smoking, should be eliminated from the donor “pool”.

A donor preferably has a bloodline that is 80% or greater of a single race or ethnicity, preferably 90% or greater, more preferably 95% or greater, and most preferably essentially 100%.

A donor is preferably of a young age since their skin and fibroblasts are at an optimal state of life. Such an age range can vary based on ethnicity. It is believed that the preferred age range is from 18 to 35, more preferably 18 to 30 years of age. However, it is envisioned that the age range can have a lower limit, as low as permitted by applicable law where the donor resides and where the biopsy occurs.

In contrast, a recipient is an individual having skin onto which the product of the present disclosure is applied or administered, and preferably skin with one or more of the skin conditions discussed herein. A recipient is not a source of the product, mixture, or any individual fibroblasts, and thus is not subject the same scrutiny discussed above for a donor. A recipient derives the benefit or characteristic or combination of skin characteristics as discussed herein.

A Caucasian recipient is defined as an individual who self-identifies as being genetically most identified as having European genetic background and typically present with Fitzpatrick skin types I-IV.

An African recipient is defined as an individual who self-identifies as being genetically most identified as having sub-Saharan genetic background and typically present with Fitzpatrick skin types IV-VI.

An Asian recipient is defined as an individual self-identifies as being genetically identified as having Asian genetic background and typically present with Fitzpatrick skin types III-V.

The following terms used in this application (“as used herein”) are defined as follows. Skin pigmentation means skin color imparted by the deposition of various melanin pigments in the skin. Skin pigmentation includes hyper-pigmentation, hypo-pigmentation, de-pigmentation, and uneven pigmentation. Hyper-pigmentation means areas of uneven pigmentation where portions of the skin appear darker or more pigmented than the common or background color. Hypo-pigmentation means areas of uneven pigmentation where portions of the skin appear lighter or less pigmented than the common or background. De-pigmentation means skin devoid of melanin pigment. Uneven pigmentation means areas of the skin with mixed or mottled pigmentation, and a random or non-contiguous pattern of normal and hypo and hyper pigmentation.

As used herein, wound healing means the ability of the skin to self-direct and regulate its own repair under the influence of locally produced and disseminated proteins, cells that comprise the dermal matrix, and other biological or cellular molecules. Cells that comprise the dermal matrix include fibroblast, blood vessel endothelial cells, blood cells (neutrophils and lymphocytes), lymphatic cells, macrophages, and mast cells. A wound can be acute or chronic. Acute means that the wound heals quickly on its own. Chronic means that the wound heals slowly and often requiring treatment.

As used herein, inflammation means the response of skin to an internal or external stimulus that may or may not cause explicit visual injury. Once initiated, the response to the stimulus is inflammation. It is controlled or regulated by locally produced proteins.

Inflammation includes reduction and prevention. As used herein, reduction means the limiting the cascade of proteins that are pro-inflammatory. This also includes the process of reversing the effects of inflammation. As used herein, prevention means the use of biologic molecules to reverse, without visual effect on the surface of the skin, the effects of internal or external (environmental) insults that may induce pro-inflammatory cascades of biological proteins and other cellular products.

As used herein, dermal matrix means the mix of one or more collagen, elastin, hyaluronic acid, proteoglycans, and other macromolecules that creates the reticular or basement layer of the dermis. Dermal matrix includes wrinkles, aging, scarring and thinning.

These following terms used in this application are defined as. Wrinkles means the uneven surface of the skin caused by sub-sufficient levels of moisture in the epidermis or damage to the reticular dermis often caused by internal or external inflammatory stimulus. Pre-mature aging means the appearance of the surface of skin marked by wrinkles, sagging, uneven pigmentation and/or superficial blood vessel damage, making the appearance of age to appear older than the chronological age of the individual. Scarring means the response to severe injury of the skin in which the deposition of collagen and other dermal macromolecules produce an uneven surface that makes it distinctly differentiated from the surrounding skin. Thinning means the reduced capacity of the skin to fully repair and regenerate resulting in a reduced depth and density of the dermal and epidermal layers of the skin.

As used herein, scar prevention means reduction of the healing process that allows abnormal amounts of collagen to be deposited in a random, not basket weave, pattern that typifies the surrounding or undamaged skin. Scar prevention includes prevention of a keloid scar, a hypertrophic scar, an atrophic scar, a contracting scar, a hyper-pigmented scar, and a hypo-pigmented scar. As used herein, keloid scar is a form of hypertrophic scarring in which the excessive production of collagen continues to grow indefinitely into large raised lesions that extend outside the area of the initial wound. As used herein, a hypertrophic scar is a scar resulting from the overproduction and deposition of collagen during the healing of a serious wound that results in the resulting scar to be raised over the surface of the surrounding skin. As used herein, atrophic scar is a scar that has an appearance that is depressed below the surface of the surrounding skin. Atrophic scar is typically caused by an inflammatory process that damages the surrounding support tissue. As used herein, hyper-pigmented scar is a scar with a degree of pigmentation that is higher or darker than the surrounding skin. As used herein, hypo-pigmented scar is a scar with a degree of pigmentation that is less or lighter than the surrounding skin.

As used herein, epidermis means the layer of the skin that covers the body and forms a protective membrane, layer, or covering over the body. The epidermis includes a layer known as the stratum corneum. The epidermis has significant role in immunity, and contains the melanin pigment that determines skin color. Epidermis includes pores, skin texture and barrier function. As used herein, a pore is an anatomical structure of the skin created by the sebaceous follicle, and serves as a duct for passage of sweat and/or sebum. A pore appears as an unfilled or filled opening in the surface of the skin. As used herein, skin texture means the normal topography and glyphic pattern apparent on the surface of the skin. Skin texture is influenced by the perceived moisture level, softness, resilience, and elasticity of the skin. As used herein, barrier function means the ability of the skin to regulate: the degree of moisture permeability, the protection from external (environmental) insults, and the immune functions protecting the skin and underlying tissues and structure.

As used herein, up regulated means increase in normalized gene expression, and/or protein production in a given cell line/type or individual relative to a control sample or a cell line/type standard, or a baseline value of an individual from an earlier time point or prior to treatment.

As used herein, down regulated means decrease in normalized gene expression, and/or protein production in a given cell line/type or individual relative to a control sample or a cell line/type standard, or a baseline value of an individual from an earlier time point or prior to treatment.

The use of fibroblast cell cultures, coculture of cells, extracts from cells or cultures, diffusible elements that form the cell culture, or culture media, either alone or with environmental conditions, are used to grow cells and induce the cells to produce their array of biologically active elements. These elements are produced by and secreted by or extracted from the cells. These cells, cultures, coculture of cells, diffusible elements form the cell culture and culture media.

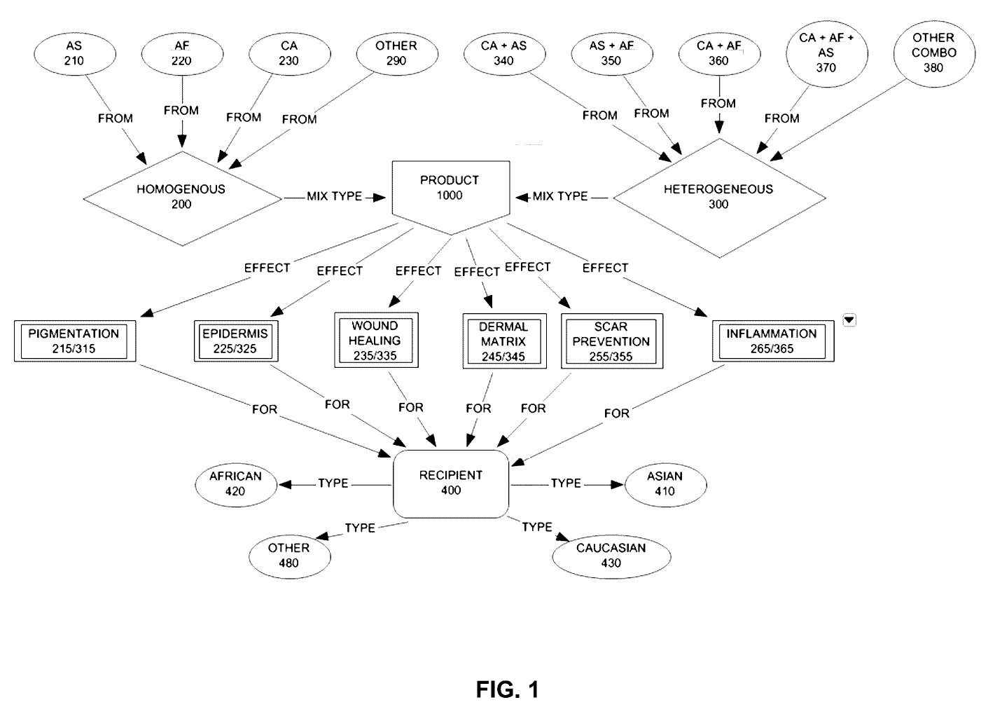

Referring to FIG. 1, the present disclosure provides a product generally represented by reference numeral 1000 that modifies cellular communication at a skin site to stimulate the production of new tissues. Specifically, the present disclosure provides a composition having proteins obtained from one or more donors, and formulating a topical product to stimulate gene expression. Specifically, the proteins can be derived from fibroblasts, cells, cocultures, cultures, diffusible elements that form the cell culture, and culture media and/or elements. For example, fibroblasts can grow and form a new, provisional extracellular matrix (ECM) by excreting collagen and fibronectin (collectively hereinafter called tissue cultures). The proteins derived from any of the above are the subject of the present disclosure. Proteins from cell signaling pathways can modulate the ‘behavior’ of fibroblasts including proliferation and migration, and this aspect is also the subject of the present disclosure.

For each embodiment of the present disclosure, recipient 400 of product 1000 is not a source or donor of the cell mixture or any individual fibroblast. Thus, recipient 400 of the proteins is not the same person whose cells were used to produce the proteins so that application to recipient 400 of the derived product 1000 is “non-autologous”.

In the present disclosure, product 400 is directed to treat a condition or provide a benefit that recipient 400 desires.

In product 1000, the proteins are blended together. However, each product 1000 is intended for a specific sex, namely women or men, so that only proteins from one sex are blended together.

The present disclosure provides a topical composition to treat a targeted skin disorder based on a desired benefit or desired characteristic. Some desired benefits or characteristics that have been found by the studies of the present disclosure are to prevent, reduce, and/or reverse visual signs of aged or prematurely aged skin (hereinafter collectively called “skin and related benefits”). Such skin and related benefits include, but are not limited to, improve or normalize pigmentation; improved barrier function; improve dermal matrix; improve the epidermis or skin quality; facilitate wound healing; minimize or prevent scarring; reduce or eliminate inflammation; treat wrinkles, stretch marks, sagging skin, scars such as surgical, traumatic, acne or chickenpox scars.

The derived products are a blend of proteins derived from certain donors, and will result in a product composition that achieves desired benefits including those benefits set forth above. The present disclosure has now proven by the studies of the present disclosure that demonstrate that certain ethnic donors have certain genes that are up-regulated gene expressions, and others that are down-regulated gene expression, and yet others that are neither.

The present disclosure contemplates that product 1000 can be either a homogenous product 200 or a heterogenous product 300.

Homogenous products 200 will first be described.

In one embodiment, homogenous product 200 is a product that has proteins derived from tissue cultures solely from a single ethnic group, such as, for example, female Asian donors 210. Analogously, in another embodiment, homogenous product 200 is derived from tissue cultures obtained solely from a group of male Asian donors 210. Likewise, homogenous product 200 is derived from tissue cultures obtained from a mixed group of male and female Asian donors 210.

In still another embodiment, homogenous product 200 is derived from tissue cultures obtained solely from a group of female African donors 220. In yet another embodiment, homogenous product 200 is derived from tissue cultures obtained solely from a group of male African donors 220. Analogously, homogenous product 200 is derived from tissue cultures obtained from a mixed group of male and female African donors 220.

In a further embodiment, homogenous product 200 is derived from tissue cultures obtained solely from a group of female Caucasian donors 230. In yet further embodiment, homogenous product 200 is derived from tissue cultures obtained solely from a group of male Caucasian donors 230. In a still yet further embodiment, homogenous product 200 is derived from tissue cultures obtained solely from a mixed group of male and female Caucasian donors 230.

Other donors 290 could also form the basis of homogenous product 200, consistent with this disclosure.

Also, it should be understood that recipient 400 can be from the same ethnic group as the donor ethnic group in homogenous product 200 or from another ethnic group, yet the product is still considered a homogenous product.

Heterogenous products 300 will now be described.

In one embodiment, heterogenous product 300 is derived from tissue cultures obtained from two or more ethnic groups. For example, one heterogenous product 300 is derived from a group 340 of female Asian and female Caucasian donors. In another embodiment, heterogenous product 300 is derived from tissue cultures obtained from a group 340 of male Asian and male Caucasian donors. Additionally, heterogenous product 300 is derived from tissue cultures obtained from a group 340 of male Asian and female Caucasian donors, or a group 340 of female Asian and male Caucasian donors, or a mixed group 340 of male and female Asian and male and female Caucasian donors.

In another embodiment, heterogenous product 300 is derived from tissue cultures obtained solely from a group 350 of female Asian and female African donors. In yet another embodiment, heterogenous product 300 is derived from tissue cultures obtained solely from a group 350 of male Asian and male African donors. Still further, heterogenous product 300 is derived from tissue cultures obtained solely from a group 350 of male Asian and female African donors, or heterogenous product 300 is derived from tissue cultures obtained solely from a group 350 of female Asian and male African donors, or heterogenous product 300 is derived from tissue cultures obtained from a mixed group 350 of male or female Asian and male or female African donors.

In a further embodiment, heterogenous product 300 is derived from tissue cultures obtained solely from a group 360 of female Caucasian and female African donors. In yet another embodiment, heterogenous product 300 is derived from tissue cultures obtained solely from a group 360 of male Caucasian and male African donors. In a still further embodiment, heterogenous product 300 is derived from tissue cultures obtained solely from a group 360 of male Caucasian and female African donors, a group 360 of female Caucasian and male African donors, or a mixed group 360 of male and female Caucasian and male and female African donors.

In a still yet further embodiment, heterogenous product 300 is derived from tissue cultures obtained solely from a group 370 of female Caucasian and female African and female Asian donors. In another embodiment, heterogenous product 300 is derived from tissue cultures obtained solely from a group 370 of male Caucasian and male African and male Asian donors. In another embodiment, heterogenous product 300 is derived from tissue cultures obtained solely from a group 370 of male Caucasian and male African and male Asian donors.

Other groups 380 of donors could also form the basis of heterogenous product 300, consistent with this disclosure.

The homogenous and heterogenous embodiments discussed above and further herein are exemplary and non-limiting, and the scope of the present disclosure will be apparent to a person having ordinary skill in the art upon understanding the present disclosure.

Homogenous product 200 and heterogenous product 300 are products that are targeted to have a desired effect imparted to the skin of recipient 400.

These targeted products are shown in FIG. 1 (as species of product 1000), with targeted homogenous products represented by a 200-series numeral and targeted heterogenous products represented by a 300-series numeral. Thus, the targeted products contemplated by the present disclosure include, at least, pigmentation 215, 315, epidermis 225, 325, wound healing 235, 335, dermal matrix 245, 345, scar prevention 255, 355, and inflammation 265, 365. Further, the proof of concept and details for the composition of such targeted products are established by the studies discussed herein.

As discussed, targeted products 215, 315, 225, 335, 245, 345, 255, 355, 265, and 365 are for an intended recipient, such as recipient 400. Recipient 400 is a female or male Asian 410, a female or male African 420, a female or male Caucasian 430, or a female or male of another ethnic origin, other 480.

As discussed in the present disclosure, the selection and weighting of cell culture proteins for product 1000 can be predicated on the particular property or pigment or other characteristic desired to be improved or imparted to the skin of recipient 400. Thus, a selection of donors of a single ethnic group, or groups of donors of different ethnic groups, and a “weighted” composition to achieve a resultant product that delivers at least one property, preferably to the skin, is possible. Moreover, one property can be improved on any person (of any ethnic background) that uses product 1000.

Thus, the data set forth herein concerning the differences in certain protein expression levels and their Biological Significance makes it clear that mixtures of homogeneous and/or heterogeneous tissue or fibroblast cultures can also be “tailored” to provide a specific desirable skin benefit or characteristic or combination of skin characteristics. Thus, “tailoring” can include a larger percentage of fibroblast cultures or derived proteins from one homogeneous group or amongst donors of the heterogeneous group. The “tailoring” can include more of one ethnic group than another ethnic group. Further, the “tailoring” can include consideration of factors based on the characteristics (noted above) desired to be obtained by the mixture. The factors include, but are not limited to, age, DNA testing, ethnic homogeneity, health, and physical beauty. For example, physical beauty could be determined by adherence to classic beauty as described by the golden ratio of physical or physiological proportions, or other similar derivations or approximations using a Fibonacci series.

What the data clearly shows is the ability to determine what fibroblasts or cells (and as stated above) of different donors in an ethnic group or different donors in different ethnic groups can be mixed to obtain the desired characteristics. This can be done at the protein level. Accordingly, the discussion herein is directed to the benefits or characteristics or combination of skin characteristics derived from proteins, and preferably derived from fibroblast cells or cell cultures of a donor to a recipient by means of a product.

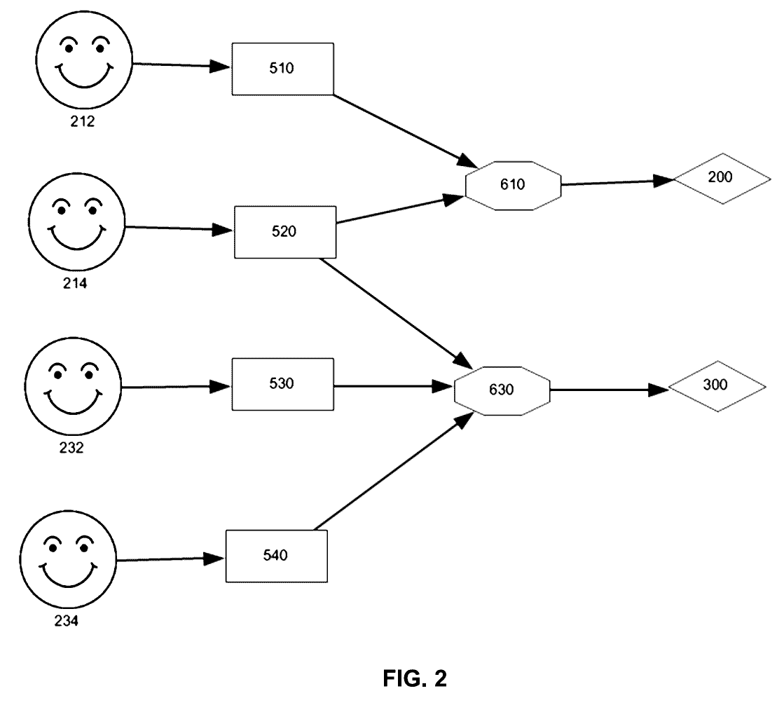

FIG. 2 is exemplary, and is by no means exhaustive, of the present disclosure. In practice, a master cell bank will be created for each individual donor in donor groups 210, 220, 230, 340, 350, 360, 370, 380, and 290.

In one example of a homogenous product 200, master cell banks 510 and 520 are created for individual donors 212 and 214 of donor group 210, respectively. A working cell bank is 610 is derived therefrom by mixing the cells produced from master cell banks 510 and 520. Homogenous product 200 of Asian donors 210 is formulated from working cell bank 610. A targeted product, such as pigmentation 215, is then derived from homogenous product 200 for a recipient 400.

In another example for a heterogeneous product 300, a master cell bank 520 is created for an individual donor 214 of donor group 210, and master cell banks 530 and 540 are created for each of individual donor 232 and 234 of donor group 230, respectively. Cells produced from master cell banks 520, 530 and 540 are blended together in a working cell bank 630. Since donor 214 is Asian, and donors 232 and 234 are Caucasian, heterogenous product 300 is created. A targeted product, such as pigmentation or pigmentation enhancing product 315 can then derived from heterogenous product 300 for recipient 400.

Product 1000 is a topical composition. The preferred topical composition is a cream, serum, or lotion. The composition can include delivery vehicles, such as liposomes and micelles. The composition can also include transport molecules, such as a protein or a macromolecule that promotes or provides molecular sledding. The use of such transport molecules enables delivery of the elements to the epidermis or dermis of a recipient. However, the composition can be parental (e.g. injectable, intravenous, or the like), delivered by device (e.g. laser, micron needle, inhaler, or the like), or an oral periodontal including a mouthwash.

The compositions comprise, in some embodiments, a pharmaceutically and/or topically acceptable vehicle to provide bulk and physical form. In other embodiments, the vehicle is hypoallergenic, as allergens and other irritating agents exacerbate pigmentation. Suitable vehicles for these benefits include, but are not limited to, cetyl alcohol, ethanol, glycerin, myristyl palmitate, polyvinyl alcohol, propylene glycol, propanol, and water, and mixtures thereof.

The topical composition can be easily prepared in any method known in the art, using the proteins herein together with at least one carrier and additives, which are commonly used in the field of preparing topical compositions. Examples of topical agents include emollients, humectants, colorants, pigments, fragrances, moisturizers, viscosity modifiers and any other topical forming agent. One or more topical agents can be included in the topical composition. The form of the topical composition can be a powder, lotion, gel, spray, stick cream, ointment, liquid, emulsion, or foam. Additional active ingredients as known in the art may also be used. Examples of the carriers may include, but are not limited to, a skin softener, a skin permeation enhancer, a colorant, an aromatic, an emulsifier, a thickener, and a solvent. Also, the topical composition may further comprise a perfumery, a pigment, a bactericidal agent, an antioxidant, a preservative and a moisturizer, and also a thickener, inorganic salts and synthetic polymer substances, for the purpose of improving physical properties.

Lists of such materials, and formulations for the creation of particular types of lotions, creams, and other such forms are widely available in the patent literature and in commercial handbooks and can be used by those skilled in the preparation of such formulations to incorporate the composition herein.

In one example, the cream or Product 1000 can be prepared by adding the composition to a general oil-in-water (O/W) cream base. The cream may further comprise a perfumery, a chelating agent, a pigment, an antioxidant, and a preservative, and also synthetic or natural materials, minerals, and vitamins, for the purpose of improving physical properties.

Product 1000 can have other biologically compatible constituents in addition to the proteins discussed above. These constituents can include one or more antioxidants, polypeptides, vitamins, plant extracts, materials derived from plant stem cells, oils, preservatives, thickening agents, ceramides, skin lighteners, exfoliants, anti-aging and anti-wrinkle agents, sunscreens, skin barrier repair agents, moisturizing ingredients, essential fatty acids, humectants, emollients, solvents, surfactants, emulsifiers, fillers, polymers, buffers, temperature regulating agents, and the like, and combinations thereof.

These constituents can comprise the delivery vehicle in the topical product. It is envisioned that the vehicle can include water. Further, the vehicle will be up to 99 wt. % of the resultant product.

It is envisioned that all the cells in the epidermis and dermis (including sub dermis) can be impacted by a topical formulation. Certain delivery vehicles can have an impact even deeper than the sub dermis.

The present disclosure has proven that proteins derived from working cell bank 610 or 630 applied in a composition to the skin of recipient 400 can have one or more of the specific effects discussed below.

In one mechanism of the embodiments of the present disclosure, the protein itself can be absorbed and utilized directly. For example, collagen can be deposited through the skin to be used as matrix structural glycoprotein or other macromolecular ground substance. Alternately MMP-1 can be absorbed to degrade collagen.

In another mechanism of the embodiments of the present disclosure, the protein can stimulate future gene expression changes. For example, cells detecting excessive amounts of deposited/delivered pro-inflammatory proteins may stimulate anti-inflammatory gene responses. It may also create a positive feedback loop that can ‘supercharge’ more pro-inflammatory protein production. Alternatively, it can bind a specific membrane site and initiate a second messenger response by activating gene or protein synthesis.

In yet another mechanism of the embodiments of the present disclosure, a protein can bind to active sites or cellular receptors. This can either halt a process by tying up all available cellular machinery or cause conformational changes in the cells and/or cellular network.

In still another mechanism of the embodiments of the present disclosure, tProtein can be broken down and subsequent components can be utilized to fuel skin/cellular process.

In yet still another mechanism of the embodiments of the present disclosure, tProtein can be used to replace “non-functional” or “mutated” proteins and restore proper cellular function/processes. Yet further, tProtein can also replace “functional” with “mutated/nonfunctional” or variant proteins.

The present disclosure contemplated different combinations and applications of the embodiments discussed herein.

TYK2 directly regulates IL-22 dependent inflammation and epidermal hyperplasia. TYK2 deficiency has multiple effects leading to reduced immune response and increased infection (viral and mycobacterial). The Asian population has reduced levels of TYK2. Thus, supplementation of TYK2 protein from African cells (who produce greater amounts of TYK2 than the other groups) could boost immune response and/or lower infection rates. Such a formulation or composition can include proteins derived from African donor group 220 in a homogenous product, or proteins derived from Caucasian and African donor group 360.

Asian skin has lower amounts of GDF3 that is a negative regulator of TGFB, which is pro-fibrotic. Application of African and/or Caucasian GDF3 can assist to decrease the rate/amount of scarring or keloid formation. Such a formulation or composition would thus include cells derived from donors of African donors 220 and Caucasian donors 230 in a homogenous product 200, or Caucasian and African donors 360, in a heterogeneous product 300.

Asian skin demonstrates a lower concentration of Visfatin enzyme, which is anti-fibrotic. Supplementation of this enzyme could decrease scarring, keloid formation and/or psoriasis. Thus, a targeted product or composition would include cells derived from donors of African donors 220 and Caucasian donors 230 in a homogenous product 200, or Caucasian and African donors 360, in a heterogeneous product 300.

Asian skin has lower levels of VitaminK1 which when applied topically can suppress pigmentation and improve wound healing. Addition of Caucasian VITK1 could function as a pigment lightener. A targeted product or composition 300 would have cells derived from donors of donors 230 in a homogenous product 200 and cells derived from donors of Caucasian and African donors 360.

In one embodiment, to treat an Asian recipient for the epidermis, a product 1000 has TSH. A homogeneous product 200 is derived from African donors 220. A heterogeneous product 300 is derived from Caucasian and African donors 360. Heterogeneous product 300 has a ratio of African Donors to Caucasian donors that is about 50% African Donors to 50% Caucasian donors, 60% African Donors to 40% Caucasian donors, 70% African Donors to 30% Caucasian donors, 80% African Donors to 20% Caucasian donors or 90% African Donors to 10% Caucasian donors. About in this paragraph means plus or minus 8%, preferably plus or minus 5%, and most preferably, plus or minus 3%, the total not exceeding 100%.

In another embodiment, to treat an Asian recipient for skin pigmentation, a product 1000 has Vitamin K1. A homogeneous product 200 is derived from Caucasian donors 230.

Caucasian skin produces more EDAR protein, which contributes to dryness and eczema through sebaceous gland interaction. Application of EDAR might be a potential treatment for oily skin. A targeted product or composition would have cells derived from donors of donors 230 in a homogenous product 200. Another targeted product or composition 300 would have cells derived from donors of Caucasian and Asian donors 340, or Caucasian and Asian and African donors 370.

Other formulation components and considerations for product 1000 are contemplated. For anti-Scarring, Follistatin, MMP7, MMP1, GDF3, Visfatin, TPA, Vasopressin, MMP10, and Pro-MMP13 are the potential proteins that when applied directly in some combination(s) have been shown to be direct components in scar and/or keloid formation. Thus, a target product would have proteins from a particular ethnic group in which these proteins are expressed, either up-regulated or down regulated based on the desired effect.

Likewise, for wound healing, the proteins TYR10, RBP4, uPA, bFGF, MMP7, IL24, Latent TGFB1, TPA, TPM1, Thrombin, IL-19, MMP8, MBL, NM23-H1/H2, NOV/CCN3, IL6, PYK2, Pro-MMP13 are considered important. Accordingly, a target product would have proteins from a particular ethnic group or groups in which these proteins are expressed, based on the desired effects.

Thus, a target product would have proteins from a particular ethnic group in which these proteins are expressed as set forth below.

For skin pigmentation, the proteins SHBG, DTK, GDNF, and Vitamin K dependent protein have been found to have a positive remedial effect.

For anti-Inflammatory effects, IL28A, Follistatin, MMP8, FIH, SERPINA12, and SSTR2 are useful.

For anti-aging effects, the effective proteins are NRG2, Kallikrein 6, RBP4, bFGF, Kallikrein 14, Kallikrein 8, Latent TGFB1, VDUP1, INSL3, Thrombin, Trappin-2/Elafin, GRP75, PARK7, Mammaglobin A, TMEFF1/Tomoregulin1.

Anti-Oxidant Enzymes include A1M and GPX3.

An anti-aging/wrinkles formulation has one or more of: RBP4, bFGF, Latent TGFB1, INSL3, Trappin-2, GRP75, PARK7, Mammaglobin A, TMEFF1/Tomoregulin1, A1M, and GPX3.

These proteins have certain benefits. For example, RBP4 is the main carrier of retinol. bFGF stimulates the growth of fibroblasts. Latent TGFB1 stimulates elastic fiber production. INSL3 reduces skin wrinkling, enhance skin appearance and improve barrier function. Trappin-2 facilitates inhibition of elastin breakdown. GRP75 prevents wrinkles and supports collagen production. PARK7 stimulates Glycation repair. Mammaglobin is a boosts barrier function. TMEFF1/Tomoregulin1 inhibit BMP signaling and generally exhibit anti-aging properties. Al M is a radical scavenger and heme binder. GPX3 is an anti-oxidant.

An anti-scarring formulation could have Follistatin. Follistatin is an activin antagonist that is anti-fibrotic and anti-inflammatory. Alternatively, or in combination, such an anti-scarring formulation could have Visfatin, which is an anti-fibrotic via enhancing inflammatory response. MMP1, MMP7, MMP10, and MMP13 assist extracellular matrix remodeling and degradation of structural components of skin. TPA has a role in wound healing and is anti-fibrotic.

A pigment lightening formulation could include SHBG. With age and decreased estrogen levels, SHBG decreases. This can lead to higher testosterone levels and pigment dyschromia as well as unwanted facial hair.

A pigment lightening formulation could also have Vitamin K dependent protein S for suppression of pigmentation.

A wound healing formulation could include: TYRO10, RBP4, uPA, bFGF, MMP1, MMP7, MMP8, Latent TGFB1, TPA, TPM1, Thrombin, IL19, NM23-H1/H2, NOV/CCN3, IL6, and PYK2.

TYRO10 mediates fibroblast migration and contributes to cutaneous wound healing. RBP4 is a main carrier of Retinol. uPA is a plasminogen activator that is a key regulator of wound healing processes. bFGF stimulates growth of fibroblasts. MMP1, MMP7, MMP8 facilitate the matrix remodeling, which is a phase of wound healing. Latent TGFB1 stimulates elastic fiber production. TPA is a plasminogen activator, has a role in wound healing, and is anti-fibrotic. TPM1 facilitates cell migration during the wound healing process. Thrombin mediates fibroblast proliferation. IL19 promotes wound healing by increasing other growth factor expression (KGF). NM23-H1/H2 is implicated in wound healing. NOV/CCN3 effects NOTCH1 mediated signaling that helps wound healing process. IL6 is required for normal wound healing. PYK2 promotes re-epithelialization.

It is envisioned that product 1000 will be proportioned based on ratios of differential protein expressions. In some embodiments, formulation methods involve a 2 to 1 to 1 ratio of a first protein to second protein to a third protein C. Further, product 1000 has a concentration or an amount of 0.0001 weight percent (wt %) to 10.0 wt % of the actives (or proteins), preferably 0.001 wt. % to 5.0 wt. %, and most preferably, 0.001 wt. % to 1.0 wt. %, based on the total weight of the product composition.

The method of application of the product to the skin will be by any topical application method known in the art. It is envisioned that the application will be administered once daily. However, it is also envisioned that the application can be twice daily if the desired benefit, such as scar healing, will tolerate repeated applications in a day. Also, the recommend number of applications per day and the period of time of application will vary based on the desired benefits of the product that will be imparted to the recipient.

Further, it is envisioned that the application of the product can continue for prolong periods of time.

Different embodiments of product 1000 can be used sequentially, mirroring the phases of wound healing. For example, a first embodiment of product 1000 that is anti-inflammatory is administered at a first time. At a second time, a second embodiment of product 1000 that is anti-hyperpigmentation is administered. At a third time, a third embodiment of product 1000 that is anti-scarring is administered.

For acute injuries, it is desirable to begin treatment as quickly as possible after the injury occurs. For example, to treat an acute injury such as a sunburn, an thermal burn, an abrasion, a traumatic injury or an elective procedure such as a laser treatment or surgical incision for Asian skin, Asian 410, at the first time, product 1000 is formulated to have IL28A, at the second time, product 1000 is formulated to have Vitamin K, and at the third time, product 1000 is formulated to have GDF3.

More specifically, frequent treatment for an initial healing phase (typically 1-10 days), up to 3 or more times per day, is desirable for the formulations of product 1000 that accelerate wound healing.

After the initial healing phase or re-epithelialization occurs treatment with product 1000 can change to 1-2 times per day using a formulation of product 1000 that reduce inflammations, reduce abnormal pigmentations, reduces risk of hypertrophic or atrophic scarring. For this second phase, administering to the skin can be in a single two or three in one combination of the product 1000 or alternating in the AM and PM with single or two in one combinations of product 1000. This second phase could continue typically for one to four (or more) weeks. In some cases, to prevent hyperpigmentation or prevent scarring, this second phase can continue for months or longer. Preferably, an anti-inflammatory product 1000 would cease to be administered after four weeks.

It is also envisioned that recipient 400 can be treated with a product 1000 before an elective procedure to minimize the risk of abnormal pigmentation. Treatment can be one to four weeks prior to the elective procedures. Specifically, a product 1000 formulated for wound healing will prepare the skin to heal faster. Such a product 1000 would include tretinoin and application would be daily for one to two weeks prior to the elective procedure.

Besides the targeted products discussed herein, additional products can focus or have properties that derive the following desired particular property or characteristics that include, but is not limited to, accelerating wound healing, improving, reducing or improving stretch marks, reducing bruising, improving sebum production, hair growth, enhancing or modulating skin firmness or smoothness, the appearance of skin, the beauty of skin, more fullness of the skin, skin tone, skin elasticity, reduced scarring, reduced wrinkles, reduced pore size, response to inflammatory stimulus, minimizing bruising, ability to retain moisture, propensity to produce new vasculature and deliver nutrients and skin thickness or density, improve response to injury or free-radical damage, reduced scarring, improved wound healing, skin disease prevention, or combination of positive skin characteristics that will be apparent to those of skill in the art. Also, the present disclosure can be used to treat ‘damaged’ skin from environmental damage, aging or disease. Further, the present disclosure can be used in veterinary applications. The present disclosure can be used as adjunctive care for treatment of skin diseases including but not limited to acne, atopic dermatitis, rosacea and psoriasis.

It is envisioned that although discussed with respect to skin, the present disclosure applies to nails, and hair. For example, the nail plate, the hard part of the nail, made of translucent keratin protein, undergoes bio-processes analogous to the skin. Likewise, hair is a protein filament that grows from follicles in the skin, and is primarily comprised of protein, and notably keratin.

The present disclosure also applies to a commercial production of an artificial gene that can be tailored to specific needs. An envisioned exemplary technique follows. Techniques include sequencing of proteins or DNA from desirable genes or gene products or complete synthetic construction of de novo gene sequences. Once a desired double stranded DNA sequence has been identified, combination into plasmid vectors with appropriate coding and non-coding sectors, as well as promotor sequences, can occur. For example, the DNA sequence can be inserted into compatible bacteria and used as factories for replication of RNA or protein product. Sequence optimization and oligo design occurs first, followed by oligo synthesis, gene assembly, sequence verification and error correction, and finally preparation of synthetic DNA for application.

As stated above, the present disclosure describes testing and analysis to determine protein production levels in cultured fibroblast supernatant from human dermal tissue samples from three (3) different ethnic populations through protein microarray performance.

### Example 1

An assay was performed in which six (6) samples of cell culture supernatant from healthy volunteers that satisfied the ethnic ancestry requirements from each of the three (3) ethnic skin groups (Asian, African, Caucasian/European) and one concentrated media control were examined for differing protein production levels (for 1,000 human proteins) as measured by protein microarrays (AAH-BLM-1000, RayBiotech, Norcross; Ga.). The volunteers were female and ranged in age from 18 to 30 years. Although females were selected, it is believed similar results would be obtained if males were selected as volunteers, or combinations thereof. The following is an explanation of the microarray process performed by RayBiotech. The first step in using the Human L-1000 arrays is to biotinylate the primary amine of the proteins in the sample. The membrane arrays are then blocked in a fashion similar to standard Western blot, and the biotin-labeled sample is added onto the pre-printed array. The array is pre-printed with capture antibodies specific to a target protein and the proteins in the sample bind with the antibodies on the array during the incubation period. After incubation using an HRP-Conjugated Streptavidin, the signal can be visualized by chemiluminescent methods and quantified. Images of the arrays are generated and analyzed for strength of signal.

The following is an analysis of the results.

The samples were accounted for as follows. Nineteen (19) samples arrived, de-identified and coded to the testing facility. All samples performed within manufacturer's limitations and data tables and array images were generated.

Data was derived including array Images. Individual images of each sample array following completion by coded identification number (sample groups are designated by prefix: As=Asian, Af=African, and Ca=Caucasian). The amount of produced protein can be visualized through the size and density of the spot generated on the array. Each array has the same proteins in the same locations for ease of visual reference.

The following are Data Tables.

Table 1 is a list of average production levels for each sample ethnic group for all proteins on the protein microarray (AAH-BLM-1000). An Asterisk (“*”) marks protein levels indicating Biological Significance (greater than a twofold increase/decrease relative to comparator sample; in this case, the Control=conditioned media). The three sample groups were As=Asian, Af=African, and Ca=Caucasian.

TABLE 1

| | CON- | AS | AF | CA |
|---|---|---|---|---|
| | TROL | Average | Average | Average |
| Positive | 68,183.33 | 68,097.33 | 68,097.33 | 68,183.33 |
| Control | | | | |
| Neg | 924.62 | 718.07 | 600.39 | 837.75 |
| 6Ckine | 129.67 | 1.04* | 87.80 | 156.30 |
| Activin A | 162.69 | 1.04* | 26.95* | 252.29 |
| Activin B | 184.35 | 1.04* | 134.33 | 387.17* |
| Activin C | 319.16 | 41.87* | 275.53 | 982.78* |
| Activin | 301.83 | 78.60* | 370.14 | 898.16* |
| RIA/ALK-2 | | | | |
| Activin | 177.85 | 55.59* | 328.09 | 814.56* |
| RIB/ALK-4 | | | | |
| Activin | 318.07 | 132.49* | 613.17 | 962.78* |
| RII A/B | | | | |
| Activin | 390.62 | 51.76* | 582.13 | 997.10* |
| RIIA | | | | |
| Adiponectin/ | 746.32 | 166.10* | 892.57 | 1,626.73* |
| Acrp30 | | | | |
| AgRP | 448.55 | 157.54* | 590.86 | 941.67* |
| ALCAM | 501.07 | 138.10* | 523.27 | 778.31 |
| Angiogenin | 397.66 | 262.88 | 492.45 | 815.27* |
| Angiopoietin-1 | 1,187.56 | 635.44 | 889.09 | 1,739.25 |
| Angiopoietin-2 | 709.50 | 378.18 | 472.85 | 1,406.26 |
| Angiopoietin-4 | 685.14 | 249.09* | 400.68 | 1,302.65 |
| Angiopoietin- | 842.69 | 259.31* | 477.29 | 1,500.64 |
| like 1 | | | | |
| Angiopoietin- | 710.59 | 590.06* | 711.49 | 1,438.05 |
| like 2 | | | | |
| Angiopoietin- | 627.21 | 116.81* | 361.17 | 996.98 |
| like Factor | | | | |
| Angiostatin | 1,071.70 | 216.87* | 711.23 | 1,343.92 |
| APJ | 536.26 | 11.82* | 184.92* | 403.49 |
| AR | 661.86 | 37.18* | 223.18* | 441.40 |
| (Amphiregulin) | | | | |
| APRIL | 823.74 | 116.65* | 326.49* | 812.58 |
| Artemin | 557.37 | 199.55* | 66.39* | 393.26 |
| Axl | 628.84 | 272.47* | 85.71* | 400.63 |
| B7-1/CD80 | 801.54 | 228.52* | 16.48* | 449.74 |
| BAFF R/ | 648.33 | 107.81* | 1.01* | 617.99 |
| TNFRSF13C | | | | |
| BCMA/ | 1.08 | 67.49* | 208.10* | 194.35* |
| TNFRSF17 | | | | |
| BD-1 | 129.12 | 65.17 | 382.42* | 402.77* |
| BDNF | 339.73 | 75.17* | 613.54 | 834.53* |
| beta-Catenin | 158.36 | 56.20* | 273.78 | 391.54* |
| BAX | 90.68 | 552.02* | 357.12* | 759.17* |
| beta-NGF | 199.51 | 358.85* | 478.62* | 897.43* |
| BIK | 5,551.78 | 7,225.66 | 9,648.28 | 14,321.51* |
| BLC/BCA-1/ | 330.52 | 557.36 | 909.49* | 1,321.99* |
| CXCL13 | | | | |
| BMP-2 | 331.61 | 244.58 | 621.09 | 1,045.93* |
| BMP-3 | 612.05 | 409.46 | 862.35 | 1,394.85* |
| BMP-3b/ | 450.17 | 304.92 | 575.64 | 916.69* |
| GDF-10 | | | | |
| BMP-4 | 492.40 | 466.21 | 819.89 | 1,234.96* |
| BMP-5 | 352.72 | 442.04 | 607.76 | 911.57* |
| BMP-6 | 699.76 | 767.21 | 1,230.53 | 1,785.35* |
| BMP-7 | 500.52 | 425.00 | 642.05 | 925.88 |
| BMP-8 | 522.18 | 269.31 | 594.91 | 975.93 |
| BMP-15 | 609.35 | 89.63* | 531.69 | 768.08 |
| BMPR-IA/ | 803.71 | 201.22* | 685.58 | 865.25 |
| ALK-3 | | | | |
| BMPR-IB/ | 812.91 | 126.71* | 614.33 | 753.41 |
| ALK-6 | | | | |
| BMPR-II | 679.19 | 122.46* | 506.20 | 587.06 |
| BTC | 643.99 | 99.02* | 385.47 | 319.45* |
| Cardiotrophin- | 679.19 | 52.57* | 330.43* | 721.03 |
| 1/CT-1 | | | | |
| CCL14/ | 482.66 | 74.14* | 187.07* | 236.13* |
| HCC-1/ | | | | |
| HCC-3 | | | | |
| CCL28/VIC | 491.86 | 23.82* | 1,115.09* | 743.42 |
| CCR1 | 642.91 | 38.55* | 369.10 | 357.10 |
| CCR2 | 560.62 | 43.11* | 297.77 | 324.95 |
| CCR3 | 245.52 | 21.27* | 275.09 | 319.12 |
| CCR4 | 181.10 | 221.30 | 159.21 | 306.58 |
| CCR5 | 71.19 | 143.29* | 192.98* | 313.52* |
| CCR6 | 111.26 | 122.49 | 321.72* | 660.49* |
| CCR7 | 437.72 | 452.52 | 841.57 | 1,092.91* |
| CCR8 | 1,018.10 | 598.45 | 1,257.90 | 1,677.89 |
| CCR9 | 352.72 | 211.93 | 720.76* | 746.79* |
| CD14 | 175.14 | 181.90 | 426.88* | 590.71* |
| CD27/ | 119.92 | 239.53 | 468.66* | 828.46* |
| TNFRSF7 | | | | |
| CD30/ | 111.26 | 272.19* | 446.20* | 489.25* |
| TNFRSF8 | | | | |
| CD30 Ligand/ | 438.80 | 597.57 | 908.21* | 1,295.22* |
| TNFSF8 | | | | |
| CD40/ | 262.85 | 484.17 | 672.82* | 904.00* |
| TNFRSF5 | | | | |
| CD40 Ligand/ | 659.15 | 710.35 | 1,131.54 | 1,455.18* |
| TNFSF5/CD154 | | | | |
| CD 163 | 944.47 | 1,775.95 | 2,269.77* | 3,457.06* |
| Cerberus 1 | 551.42 | 844.48 | 784.21 | 1,473.97* |
| Chem R23 | 457.75 | 689.12 | 680.21 | 1,006.74* |
| Chordin-Like 1 | 755.52 | 760.62 | 918.92 | 1,233.52 |
| Chordin-Like 2 | 609.35 | 683.02 | 931.67 | 1,299.52* |
| Csk | 2,524.28 | 3,252.64 | 5,507.31* | 5,510.66* |
| CLC | 497.82 | 539.58 | 763.91 | 990.60 |
| CNTF | 719.25 | 653.90 | 1,577.91* | 1,661.29* |
| CNTF R alpha | 1,247.11 | 897.87 | 1,679.58 | 2,264.41 |
| Coagulation | 538.96 | 435.74 | 693.02 | 747.54 |
| Factor III/ | | | | |
| Tissue Factor | | | | |
| CRIM 1 | 815.08 | 527.66 | 766.91 | 819.49 |
| Cripto-1 | 581.73 | 291.49 | 497.48 | 510.38 |
| CRTH-2 | 658.07 | 175.22* | 475.55 | 488.39 |
| Cryptic | 584.98 | 161.00* | 470.95 | 550.17 |
| CTACK/CCL27 | 871.38 | 440.23 | 903.29 | 1,020.30 |
| CTGF/CCN2 | 433.93 | 178.17* | 445.48 | 270.78 |
| CTLA-4/CD152 | 419.31 | 165.58* | 588.91 | 228.33 |
| CV-2/ | 108.01 | 18.99* | 450.71* | 45.32* |
| Crossveinless-2 | | | | |
| CXCL14/BRAK | 687.85 | 730.98 | 662.88 | 799.70 |
| CXCL16 | 44.67 | 293.17* | 389.68* | 380.72* |
| CXCR1/IL-8 RA | 353.26 | 382.73 | 735.14* | 658.46 |
| CXCR2/IL-8 RB | 328.90 | 485.84 | 763.62* | 696.30* |
| CXCR3 | 300.21 | 318.38 | 588.38 | 438.89 |
| CXCR4 (fusin) | 353.26 | 393.13 | 1,955.26* | 615.73 |
| CXCR5/BLR-1 | 465.33 | 310.15 | 650.04 | 650.99 |
| CXCR6 | 438.26 | 431.89 | 748.94 | 1,253.17* |
| D6 | 216.83 | 435.25* | 652.24* | 453.43* |
| DAN | 686.77 | 1,597.31* | 2,464.56* | 2,888.04* |
| DANCE | 454.51 | 511.09 | 737.37 | 709.65 |
| DcR3/ | 622.34 | 927.20 | 1,286.49* | 1,462.71* |
| TNFRSF6B | | | | |
| Decorin | 461.00 | 1,900.72* | 4,089.23* | 3,161.92* |
| Dkk-1 | 609.89 | 2,836.46* | 3,118.77* | 3,029.36* |
| Dkk-3 | 863.26 | 6,466.27* | 5,277.86* | 6,745.95* |
| Dkk-4 | 754.44 | 853.57 | 1,099.56 | 1,228.59 |
| DR3/ | 620.17 | 520.59 | 812.51 | 874.13 |
| TNFRSF25 | | | | |
| DR6/ | 617.47 | 587.92 | 1,141.45 | 1,039.20 |
| TNFRSF21 | | | | |
| Dtk | 732.24 | 1,115.96 | 2,856.32* | 3,481.06* |
| EDA-A2 | 813.45 | 45,352.87* | 86,934.18* | 102,337.42* |
| EDAR | 578.49 | 1,715.75* | 2,752.31* | 3,558.44* |
| EDG-1 | 753.36 | 517.24 | 1,101.42 | 980.91 |
| EGF | 1,057.08 | 804.21 | 1,455.25 | 1,379.46 |
| EGF R/ | 596.89 | 572.81 | 829.08 | 668.71 |
| ErbB1 | | | | |
| EG-VEGF/ | 967.75 | 477.34* | 889.30 | 782.98 |
| PK1 | | | | |
| EMAP-II | 595.81 | 282.08 | 724.99 | 537.13 |
| ENA-78 | 559.54 | 334.91 | 801.05 | 403.78 |
| Endocan | 671.61 | 1,233.80 | 1,919.73* | 1,631.78* |
| Endoglin/ | 511.35 | 348.01 | 878.27 | 333.41 |
| CD105 | | | | |
| Endostatin | 1,248.20 | 2,031.72 | 3,492.62* | 2,175.26 |
| EN-RAGE | 515.14 | 635.45 | 620.78 | 729.35 |
| Eotaxin/ | 1,498.32 | 559.22* | 563.75* | 583.23* |
| CCL11 | | | | |
| Eotaxin-2/ | 397.66 | 545.51 | 578.71 | 525.92 |
| MPIF-2 | | | | |
| Eotaxin-3/ | 272.05 | 475.03 | 437.37 | 499.31 |
| CCL26 | | | | |
| Epiregulin | 510.81 | 956.19 | 710.28 | 1,017.19 |
| ErbB2 | 516.22 | 687.68 | 643.10 | 779.11 |
| ErbB3 | 568.20 | 505.73 | 557.91 | 902.36 |
| ErbB4 | 710.59 | 929.88 | 961.35 | 1,377.37 |
| Erythro- | 1,011.61 | 1,328.05 | 1,363.61 | 1,787.40 |
| poietin | | | | |
| E-Selectin | 434.47 | 621.02 | 822.96 | 766.18 |
| Endothelin | 4,035.32 | 3,698.55 | 3,500.99 | 6,526.71 |
| FADD | 471.83 | 861.06 | 1,015.13* | 1,006.16* |
| FAM3B | 1,191.89 | 2,408.77* | 2,220.45 | 2,201.66 |
| Fas/ | 659.15 | 963.98 | 1,126.12 | 1,045.59 |
| TNFRSF6 | | | | |
| Fas Ligand | 869.22 | 1,041.61 | 1,047.34 | 1,297.45 |
| FGF Basic | 892.50 | 913.25 | 1,070.88 | 1,014.87 |
| FGF-BP | 812.91 | 743.57 | 822.53 | 862.25 |
| FGF R3 | 837.82 | 896.54 | 921.54 | 922.49 |
| FGF R4 | 717.63 | 823.16 | 1,041.09 | 1,105.28 |
| FGF R5 | 674.85 | 1,554.38* | 2,334.15* | 2,276.60* |
| FGF-4 | 742.53 | 693.36 | 992.91 | 945.37 |
| FGF-5 | 795.05 | 569.83 | 1,207.66 | 857.43 |
| FGF-6 | 690.01 | 533.12 | 1,219.22 | 783.20 |
| FGF-7/KGF | 698.68 | 419.83 | 974.20 | 663.87 |
| FGF-8 | 684.06 | 268.04* | 928.30 | 562.73 |
| FGF-9 | 688.93 | 356.72 | 1,032.44 | 707.72 |
| FGF-10/ | 664.03 | 364.21 | 928.11 | 859.52 |
| KGF-2 | | | | |
| FGF-11 | 858.93 | 452.56 | 1,027.67 | 590.53 |
| FGF-12 | 568.20 | 313.15 | 918.76 | 531.95 |
| FGF-13 1B | 713.84 | 333.88 | 1,055.18 | 604.71 |
| FGF-16 | 760.94 | 1,063.44 | 628.74 | 929.24 |
| FGF-17 | 792.88 | 881.75 | 898.80 | 1,133.31 |
| FGF-18 | 327.28 | 542.26 | 452.27 | 506.29 |
| FGF-19 | 409.57 | 560.82 | 496.34 | 503.71 |
| FGF-20 | 482.66 | 624.20 | 715.54 | 588.27 |
| FGF-21 | 559.54 | 687.95 | 780.45 | 752.21 |
| FGF-23 | 1,106.89 | 1,228.41 | 1,178.19 | 1,927.55 |
| FLRG | 654.82 | 470.54 | 657.76 | 907.18 |
| Flt-3 Ligand | 561.70 | 580.12 | 786.25 | 588.98 |
| Follistatin | 1,096.61 | 1,517.05 | 1,970.24 | 2,259.23* |
| Follistatin-like 1 | 3,372.11 | 2,632.57 | 3,143.13 | 5,471.44 |
| Fractalkine | 601.77 | 725.25 | 970.37 | 855.59 |
| Frizzled-1 | 634.79 | 1,193.13 | 1,234.68 | 1,294.11 |
| Frizzled-3 | 702.47 | 927.29 | 1,281.45 | 1,096.65 |
| Frizzled-4 | 813.45 | 936.57 | 1,105.16 | 1,098.27 |
| Frizzled-5 | 1,000.24 | 976.11 | 1,221.93 | 1,295.82 |
| Frizzled-6 | 814.54 | 752.23 | 923.13 | 857.59 |
| Frizzled-7 | 875.71 | 726.64 | 837.95 | 704.22 |
| Galectin-3 | 935.27 | 1,214.96 | 1,576.53 | 1,065.51 |
| GASP-1/ | 863.26 | 774.45 | 1,194.13 | 1,008.42 |
| WFIKKNRP | | | | |
| GASP-2/ | 949.89 | 588.74 | 1,026.76 | 728.60 |
| WFIKKN | | | | |
| GCP-2/CXCL6 | 791.26 | 438.11 | 1,088.36 | 624.91 |
| GCSF | 828.61 | 332.82* | 921.43 | 491.36 |
| G-CSF R/ | 1,033.26 | 354.90* | 1,141.81 | 590.72 |
| CD 114 | | | | |
| GDF1 | 1,111.76 | 483.82* | 1,610.55 | 823.26 |
| GDF3 | 3,578.92 | 4,531.00 | 11,028.23* | 8,524.73* |
| GDF5 | 1,168.07 | 1,085.02 | 2,955.55* | 1,591.81 |
| GDF8 | 709.50 | 438.75 | 1,003.97 | 528.23 |
| GDF9 | 1,021.89 | 550.33 | 1,530.81 | 969.54 |
| GDF11 | 782.05 | 326.46* | 1,788.77* | 916.98 |
| GDF-15 | 1,169.15 | 1,695.58 | 1,474.42 | 1,852.05 |
| GDNF | 852.43 | 1,048.30 | 855.87 | 1,229.68 |
| GFR alpha-1 | 706.26 | 806.45 | 782.02 | 893.06 |
| GFR alpha-2 | 486.99 | 791.81 | 890.19 | 984.65* |
| GFR alpha-3 | 773.93 | 1,090.87 | 1,645.07* | 1,332.54 |
| GFR alpha-4 | 600.14 | 717.99 | 873.03 | 1,197.73 |
| GITR/TNFRF18 | 579.57 | 672.36 | 834.29 | 1,503.39* |
| GITR Ligand/ | 684.60 | 830.03 | 1,169.62 | 2,155.31* |
| TNFSF18 | | | | |
| Glucagon | 750.11 | 668.12 | 861.19 | 889.11 |
| Glut1 | 734.95 | 669.05 | 831.57 | 841.10 |
| Glut2 | 963.42 | 1,011.25 | 1,528.02 | 1,533.56 |
| Glut3 | 718.71 | 841.86 | 1,552.83* | 1,146.30 |
| Glut5 | 823.74 | 1,037.65 | 1,527.07 | 1,228.58 |
| Glypican 3 | 5,638.95 | 8,992.11 | 15,523.37* | 15,039.53* |
| Glypican 5 | 1,064.12 | 1,177.50 | 2,206.01* | 1,877.10 |
| GM-CSF | 942.31 | 776.65 | 1,291.47 | 1,212.68 |
| GM-CSF R alpha | 1,122.05 | 807.13 | 1,555.05 | 1,316.34 |
| Granzyme A | 950.43 | 798.26 | 1,362.27 | 1,141.08 |
| GREMLIN | 3,976.85 | 3,596.94 | 6,204.15 | 6,163.27 |
| GRO | 874.63 | 756.08 | 1,429.54 | 932.18 |
| GRO-a | 842.15 | 427.97 | 1,064.41 | 683.69 |
| Growth Hormone | 1,610.39 | 818.75 | 1,920.17 | 1,539.95 |
| (GH) | | | | |
| Growth Hormone | 1,234.12 | 875.68 | 1,958.46 | 1,421.15 |
| R (GHR) | | | | |
| HB-EGF | 933.64 | 496.70 | 1,334.73 | 851.17 |
| HCC-4/CCL16 | 1,837.24 | 938.99 | 1,451.66 | 1,266.74 |
| HCR/CRAM-A/B | 1,137.75 | 611.82 | 1,456.60 | 963.83 |
| Hepassocin | 1,105.81 | 527.18* | 1,257.02 | 939.06 |
| GLO-1 | 948.80 | 377.68* | 954.76 | 689.95 |
| HGF | 768.52 | 307.61* | 987.65 | 536.86 |
| HGFR | 860.01 | 257.00* | 1,025.92 | 280.02 |
| HRG-alpha/ | 878.42 | 1,668.37 | 675.14 | 1,100.20 |
| NRG1-alpha | | | | |
| HRG-beta 1/ | 992.66 | 1,820.68 | 1,078.54 | 1,636.71 |
| NRG1-beta 1 | | | | |
| HVEM/ | 814.54 | 1,446.50 | 954.58 | 1,290.55 |
| TNFRSF14 | | | | |
| I-309 | 751.19 | 1,100.69 | 905.79 | 1,023.92 |
| ICAM-1 | 858.39 | 1,142.13 | 1,189.69 | 1,381.08 |
| ICAM-2 | 1,928.19 | 3,678.69 | 3,997.86* | 4,048.00* |
| ICAM-3 (CD50) | 597.43 | 815.45 | 847.23 | 3,273.74* |
| ICAM-5 | 680.81 | 853.84 | 996.91 | 3,453.60* |
| IFN-alpha/ | 760.94 | 822.80 | 980.25 | 1,248.04 |
| beta R1 | | | | |
| IFN-alpha/ | 743.07 | 943.90 | 1,274.51 | 1,106.74 |
| beta R2 | | | | |
| IFN-beta | 767.43 | 946.59 | 1,351.76 | 985.88 |
| IFN-gamma | 784.22 | 938.59 | 1,450.03 | 1,107.31 |
| IFN-gamma R1 | 1,054.38 | 1,213.84 | 1,667.96 | 1,360.48 |
| IGFBP-1 | 1,446.89 | 1,990.44 | 2,757.24 | 2,804.42 |
| IGFBP-2 | 1,230.33 | 11,164.80* | 22,591.35* | 23,988.15* |
| IGFBP-3 | 1,211.38 | 4,239.29* | 5,899.29* | 7,880.22* |
| IGFBP-4 | 1,045.17 | 1,306.00 | 2,071.58 | 1,725.43 |
| IGFBP-6 | 1,520.52 | 2,899.78 | 4,019.98* | 4,500.41* |
| IGFBP-rp1/ | 11,808.74 | 76,254.45* | 90,204.78* | 94,029.81* |
| IGFBP-7 | | | | |
| IGF-I | 1,396.00 | 2,727.98 | 3,375.51* | 3,317.66* |
| IGF-I SR | 1,114.47 | 698.60 | 1,327.13 | 1,320.74 |
| IGF-II | 1,063.04 | 691.36 | 1,447.24 | 1,306.61 |
| IGF-II R | 1,063.04 | 761.02 | 1,690.45 | 1,400.97 |
| IL-1 alpha | 851.89 | 470.82 | 892.28 | 962.01 |
| IL-1 beta | 998.61 | 483.09* | 915.40 | 780.48 |
| IL-1 F5/ | 998.61 | 538.11 | 1,032.52 | 908.99 |
| FIL1delta | | | | |
| IL-1 F6/FIL1 | 1,609.85 | 1,150.85 | 2,492.58 | 2,461.83 |
| epsilon | | | | |
| IL-1 F7/FIL1 | 1,202.18 | 598.54* | 1,232.35 | 1,262.28 |
| zeta | | | | |
| IL-1 F8/FIL1 | 1,098.23 | 599.85 | 1,113.49 | 931.43 |
| eta | | | | |
| IL-1 F9/ | 1,138.83 | 483.57* | 1,141.34 | 719.34 |
| IL-1 H1 | | | | |
| IL-1 F10/ | 871.38 | 1,635.97 | 1,270.14 | 1,312.48 |
| IL-1HY2 | | | | |
| IL-1 R3/ | 823.20 | 1,955.99 | 865.40 | 995.25 |
| IL-1 R AcP | | | | |
| IL-1 R4/ST2 | 1,274.73 | 2,118.88 | 1,817.45 | 1,822.67 |
| IL-1 R6/ | 898.45 | 1,525.80 | 978.33 | 1,154.15 |
| IL-1 Rrp2 | | | | |
| IL-1 R8 | 1,002.94 | 1,332.27 | 1,107.02 | 1,556.52 |
| IL-1 R9 | 1,022.43 | 1,167.67 | 1,206.69 | 2,452.64* |
| IL-1 ra | 1,111.22 | 1,200.55 | 1,430.59 | 3,565.28* |
| IL-1 sRI | 821.03 | 1,025.90 | 1,353.87 | 4,205.60* |
| IL-1 sRII | 874.63 | 1,253.81 | 1,732.58 | 1,719.92 |
| IL-2 | 887.08 | 1,210.26 | 1,539.61 | 1,469.06 |
| IL-2 R alpha | 859.47 | 1,427.19 | 2,055.22* | 2,053.81* |
| IL-2 R beta/ | 772.85 | 1,432.81 | 1,731.74* | 1,822.64* |
| CD122 | | | | |
| IL-2 R gamma | 979.66 | 1,440.19 | 1,602.61 | 1,821.47 |
| IL-3 | 1,118.26 | 1,818.82 | 1,971.30 | 2,369.44* |
| IL-3 R alpha | 982.37 | 1,837.07 | 2,077.86* | 2,169.15* |
| IL-4 | 1,123.68 | 1,557.32 | 1,772.86 | 1,757.93 |
| IL-4 R | 3,284.40 | 2,700.92 | 3,384.74 | 4,704.27 |
| IL-5 | 1,240.08 | 1,682.21 | 2,004.37 | 2,070.95 |
| IL-5 R alpha | 1,489.12 | 3,137.10* | 3,075.82* | 3,982.92* |
| IL-6 | 978.04 | 1,502.19 | 1,930.21 | 2,591.63* |
| IL-6 R | 1,025.68 | 866.20 | 1,630.12 | 1,619.06 |
| IL-7 | 1,214.63 | 942.45 | 1,551.42 | 1,707.75 |
| IL-7 R alpha | 1,212.46 | 1,221.60 | 1,899.36 | 1,908.75 |
| IL-8 | 1,300.71 | 836.08 | 1,193.16 | 1,494.92 |
| IL-9 | 995.90 | 708.33 | 1,137.45 | 1,018.95 |
| IL-10 | 1,135.04 | 677.63 | 1,044.99 | 1,018.25 |
| IL-10 R alpha | 1,062.50 | 710.57 | 1,132.07 | 1,058.13 |
| IL-10 R beta | 1,111.76 | 605.73 | 986.08 | 886.46 |
| IL-11 | 1,251.99 | 699.67 | 1,003.71 | 959.33 |
| IL-12 p40 | 1,555.17 | 859.87 | 1,676.54 | 1,090.19 |
| IL-12 p70 | 1,191.89 | 1,424.88 | 1,556.92 | 1,681.00 |
| IL-12 R beta 1 | 1,076.57 | 1,411.21 | 1,352.07 | 1,740.12 |
| IL-12 R beta 2 | 2,022.94 | 4,457.84* | 6,617.74* | 7,987.21* |
| IL-13 | 2,448.48 | 2,611.50 | 2,626.23 | 4,435.29 |
| IL-13 R alpha 1 | 930.40 | 1,270.79 | 1,345.81 | 1,593.24 |
| IL-13 R alpha 2 | 892.50 | 1,314.15 | 1,352.48 | 1,931.95* |
| IL-15 | 921.73 | 1,288.79 | 1,317.45 | 1,713.47 |
| IL-15 R alpha | 1,080.90 | 1,586.74 | 1,575.32 | 2,851.23* |
| IL-16 | 1,167.53 | 1,581.88 | 1,486.05 | 1,967.21 |
| IL-17 | 1,481.54 | 1,530.49 | 1,458.11 | 2,146.07 |
| IL-17B | 1,593.61 | 1,771.99 | 1,664.38 | 2,534.18 |
| IL-17B R | 1,605.52 | 1,572.19 | 1,524.19 | 2,281.38 |
| IL-17C | 1,988.83 | 1,797.99 | 1,902.92 | 2,841.57 |
| IL-17D | 1,481.54 | 1,287.88 | 1,303.66 | 1,806.32 |
| IL-17E | 1,305.04 | 1,355.70 | 1,397.57 | 1,925.20 |
| IL-17F | 1,136.67 | 986.78 | 1,134.73 | 1,391.13 |
| IL-17R | 1,126.92 | 1,097.36 | 1,530.86 | 1,771.58 |
| IL-17RC | 2,280.65 | 2,719.20 | 4,631.07* | 5,247.83* |
| IL-17RD | 1,538.93 | 2,293.43 | 2,337.81 | 3,481.11* |
| IL-18 BPa | 1,312.08 | 996.32 | 1,569.63 | 1,678.07 |
| IL-18 R alpha/ | 1,191.35 | 879.37 | 1,461.92 | 1,427.80 |
| IL-1 R5 | | | | |
| IL-18 R beta/ | 1,242.78 | 917.24 | 1,163.68 | 1,338.43 |
| AcPL | | | | |
| IL-19 | 1,488.58 | 1,048.58 | 1,678.47 | 1,749.11 |
| IL-20 | 1,178.36 | 744.74 | 1,053.19 | 1,074.57 |
| IL-20 R alpha | 1,506.99 | 906.17 | 1,358.78 | 1,365.94 |
| IL-20 R beta | 1,235.20 | 863.38 | 1,775.17 | 1,189.55 |
| IL-21 | 991.57 | 1,359.62 | 1,013.24 | 1,326.37 |
| IL-21 R | 845.94 | 1,251.77 | 1,101.00 | 1,313.91 |
| IL-22 | 873.55 | 1,188.37 | 1,224.95 | 1,631.76 |
| IL-22 BP | 823.74 | 1,009.44 | 1,078.76 | 1,465.05 |
| IL-22 R | 783.13 | 994.00 | 917.09 | 1,365.21 |
| IL-23 | 1,063.58 | 1,488.63 | 1,315.07 | 2,084.62 |
| IL-23 R | 876.80 | 1,333.15 | 1,135.22 | 1,674.62 |
| IL-24 | 1,015.40 | 1,464.60 | 1,190.86 | 1,896.41 |
| IL-26 | 1,270.94 | 1,529.58 | 1,263.03 | 2,256.14 |
| IL-27 | 1,355.94 | 1,651.98 | 1,476.52 | 2,472.08 |
| IL-28A | 1,259.57 | 1,388.22 | 1,146.49 | 1,986.92 |
| IL-29 | 1,473.42 | 1,478.44 | 1,255.22 | 2,577.44 |
| IL-31 | 1,153.45 | 960.07 | 925.66 | 1,633.63 |
| IL-31 RA | 1,146.41 | 1,002.13 | 909.86 | 1,459.91 |
| BACE-1 | 1,196.22 | 1,108.51 | 1,040.26 | 1,572.98 |
| FACX | 1,047.88 | 996.02 | 1,014.70 | 1,394.57 |
| Insulin | 1,070.08 | 857.52 | 1,081.85 | 1,131.61 |
| Insulin R | 1,126.92 | 930.01 | 1,277.26 | 1,439.58 |
| Insulysin/IDE | 1,817.21 | 1,685.20 | 2,563.70 | 3,264.46 |
| IP-10 | 1,358.10 | 910.12 | 1,517.16 | 1,720.63 |
| I-TAC/CXCL11 | 1,892.46 | 1,099.39 | 2,026.82 | 2,420.30 |
| Kininostatin/ | 2,319.63 | 1,645.60 | 2,622.11 | 3,234.01 |
| kininogen | | | | |
| Kremen-1 | 980.75 | 851.20 | 1,145.99 | 1,321.05 |
| Kremen-2 | 1,010.52 | 1,194.89 | 1,865.48 | 1,931.15 |
| Lck | 1,020.81 | 864.75 | 1,387.45 | 1,542.06 |
| Latent | 922.82 | 4,029.66* | 7,681.24* | 6,544.87* |
| TGF-beta bp1 | | | | |
| LBP | 739.82 | 1,164.35 | 1,527.46 | 1,327.60 |
| LECT2 | 936.89 | 1,051.80 | 1,210.37 | 1,191.53 |
| Lefty - A | 864.89 | 950.60 | 1,267.29 | 1,277.01 |
| Leptin R | 988.33 | 1,196.73 | 1,412.49 | 1,880.36 |
| Leptin (OB) | 722.50 | 1,616.88 | 1,276.53 | 1,720.56* |
| LFA-1 alpha | 611.51 | 1,354.79* | 1,250.51* | 1,524.15* |
| LIF | 624.50 | 1,169.36 | 1,020.34 | 1,561.29* |
| LIFR alpha | 932.02 | 1,288.14 | 1,185.20 | 1,951.66* |
| LIGHT/TNFSF14 | 1,070.62 | 1,452.53 | 1,412.29 | 2,078.84 |
| Lipocalin-1 | 1,293.67 | 1,330.40 | 1,165.17 | 2,006.71 |
| LRP-1 | 2,188.61 | 2,890.83 | 3,069.30 | 4,717.33* |
| LRP-6 | 1,394.92 | 2,364.58 | 2,607.56 | 4,087.56* |
| L-Selectin | 1,267.69 | 1,382.26 | 1,261.10 | 1,929.25 |
| (CD62L) | | | | |
| Lipocalin-2 | 2,476.63 | 1,714.70 | 1,896.41 | 3,278.27 |
| Lymphotactin/ | 1,098.77 | 1,312.00 | 1,330.06 | 2,054.79 |
| XCL1 | | | | |
| Lymphotoxin | 1,365.14 | 994.38 | 1,087.69 | 1,611.76 |
| beta/TNFSF3 | | | | |
| Lymphotoxin | 1,200.01 | 977.19 | 1,166.55 | 1,623.79 |
| beta R/TNFRSF3 | | | | |
| MAC-1 | 1,048.42 | 862.67 | 1,163.06 | 1,504.15 |
| MCP-1 | 1,129.63 | 1,265.71 | 2,430.37* | 2,927.03* |
| MCP-2 | 1,419.82 | 928.78 | 1,394.29 | 1,690.51 |
| MCP-3 | 1,783.64 | 715.44* | 1,247.71 | 1,365.85 |
| MCP-4/CCL13 | 1,465.30 | 727.71* | 1,057.67 | 1,252.35 |
| M-CSF | 1,502.11 | 1,201.53 | 1,923.83 | 2,237.99 |
| M-CSF R | 1,508.07 | 826.40 | 1,295.48 | 1,580.06 |
| MDC | 1,169.69 | 846.17 | 1,091.48 | 1,415.72 |
| MFG-E8 | 1,131.80 | 872.70 | 1,128.30 | 1,462.37 |
| MFRP | 1,345.65 | 1,467.26 | 822.38 | 1,120.95 |
| MIF | 1,268.77 | 1,505.63 | 1,009.50 | 1,418.99 |
| MIG | 919.03 | 1,182.19 | 1,157.38 | 1,197.39 |
| MIP-1a | 1,062.50 | 1,603.22 | 1,442.09 | 2,240.14* |
| MIP-1b | 1,432.81 | 1,780.78 | 1,923.16 | 2,801.09 |
| MIP-1d | 720.33 | 957.50 | 1,033.11 | 1,025.64 |
| MIP 2 | 812.91 | 1,202.63 | 1,345.46 | 1,554.83 |
| MIP-3 alpha | 752.27 | 1,098.79 | 1,299.21 | 2,124.54* |
| MIP-3 beta | 1,536.22 | 3,864.34* | 4,525.98* | 5,350.64* |
| MMP-1 | 974.25 | 46,620.53* | 53,622.33* | 38,420.38* |
| MMP-2 | 890.87 | 2,317.71* | 2,954.82* | 2,737.83* |
| MMP-3 | 871.92 | 6,219.51* | 7,061.18* | 8,496.31* |
| MMP-7 | 1,365.68 | 1,847.91 | 2,036.66 | 2,255.43 |
| MMP-8 | 1,148.58 | 1,599.00 | 1,500.34 | 1,771.33 |
| MMP-9 | 1,063.58 | 1,463.73 | 1,136.51 | 1,667.24 |
| MMP-10 | 1,000.24 | 1,414.52 | 1,193.34 | 1,726.21 |
| MMP-11/ | 1,012.69 | 1,254.77 | 1,157.85 | 1,693.37 |
| Stromelysin-3 | | | | |
| MMP-12 | 994.82 | 1,143.14 | 979.01 | 1,512.12 |
| MMP-13 | 1,191.35 | 1,078.47 | 1,011.75 | 1,495.32 |
| MMP-14 | 1,486.95 | 916.70 | 907.97 | 1,338.15 |
| MMP-15 | 884.92 | 917.37 | 866.83 | 1,260.41 |
| MMP-16/ | 1,966.63 | 2,112.37 | 2,387.14 | 3,465.00 |
| MT3-MMP | | | | |
| MMP-19 | 1,672.65 | 1,421.14 | 1,371.97 | 1,941.23 |
| MMP-20 | 3,688.29 | 2,978.21 | 3,537.24 | 5,325.25 |
| MMP-24/ | 2,324.50 | 811.10* | 1,058.40* | 1,195.20 |
| MT5-MMP | | | | |
| MMP-25/ | 1,484.25 | 1,220.55 | 1,056.60 | 1,111.16 |
| MT6-MMP | | | | |
| Musk | 1,294.76 | 1,250.11 | 2,918.24* | 2,445.54 |
| MSP alpha Chain | 1,335.90 | 967.98 | 1,256.59 | 1,535.42 |
| MICA | 1,621.22 | 946.70 | 1,401.84 | 1,595.52 |
| NAP-2 | 1,169.69 | 806.53 | 1,202.42 | 1,087.58 |
| NCAM-1/CD56 | 1,980.71 | 1,778.31 | 1,443.41 | 2,299.71 |
| Neuritin | 1,378.67 | 1,336.50 | 972.64 | 1,591.44 |
| NeuroD1 | 909.82 | 1,149.84 | 994.24 | 1,234.65 |
| Neuropilin-2 | 789.09 | 1,196.15 | 1,099.20 | 1,328.89 |
| Neurturin | 1,096.61 | 1,375.09 | 1,208.00 | 1,520.63 |
| NGF R | 750.65 | 1,103.03 | 941.67 | 1,170.63 |
| NOV/CCN3 | 620.71 | 1,086.36 | 1,005.70 | 1,320.93* |
| NRG1 Isoform | 736.03 | 1,192.45 | 1,237.58 | 1,436.60 |
| GGF2 | | | | |
| Nidogen-1 | 1,167.53 | 3,590.00* | 4,048.72* | 4,497.40* |
| NrCam | 1,193.52 | 2,442.48* | 3,271.70* | 3,210.07* |
| NRG2 | 1,441.48 | 1,749.67 | 2,059.84 | 2,382.91 |
| NRG3 | 1,374.34 | 1,736.13 | 1,891.69 | 2,313.99 |
| NT-3 | 1,120.43 | 1,392.99 | 1,202.29 | 1,353.57 |
| NT-4 | 942.31 | 1,557.07 | 1,357.60 | 1,357.37 |
| Orexin A | 1,120.43 | 1,809.08 | 1,600.93 | 1,877.55 |
| Orexin B | 771.22 | 1,572.81* | 1,347.76 | 1,699.51* |
| OSM | 1,122.05 | 1,536.35 | 1,426.09 | 1,918.03 |
| Osteoactivin/ | 839.98 | 1,279.25 | 1,262.17 | 1,559.33 |
| GPNMB | | | | |
| Osteocrin | 822.66 | 1,153.43 | 1,176.48 | 1,445.54 |
| Osteoprotegerin/ | 974.25 | 2,120.02* | 2,149.40* | 3,157.01* |
| TNFRSF11B | | | | |
| OX40 Ligand/ | 1,029.47 | 1,051.41 | 1,275.32 | 1,572.17 |
| TNFSF4 | | | | |
| PARC/CCL18 | 1,117.72 | 1,029.19 | 1,384.60 | 1,576.61 |
| PD-ECGF | 1,084.15 | 1,043.40 | 1,267.25 | 1,343.42 |
| PDGF R alpha | 1,214.09 | 986.83 | 1,122.81 | 1,231.32 |
| PDGF R beta | 2,061.38 | 1,254.24 | 2,032.44 | 1,900.76 |
| PDGF-AA | 1,408.45 | 1,310.35 | 1,532.25 | 1,467.41 |
| PDGF-AB | 1,268.23 | 924.10 | 2,110.42 | 1,431.92 |
| PDGF-BB | 1,284.47 | 953.54 | 2,303.68 | 1,426.55 |
| PDGF-C | 1,236.29 | 1,084.41 | 2,432.17 | 1,591.44 |
| PDGF-D | 1,211.38 | 933.24 | 2,300.94 | 1,362.53 |
| PECAM-1/CD31 | 2,262.24 | 1,106.12* | 844.02* | 1,558.11 |
| Pentraxin3/ | 1,721.92 | 1,381.56 | 1,620.95 | 1,868.87 |
| TSG-14 | | | | |
| Persephin | 1,104.73 | 911.80 | 1,150.43 | 1,114.11 |
| PF4/CXCL4 | 1,026.22 | 1,244.27 | 1,435.22 | 1,387.35 |
| PIGF | 796.13 | 956.91 | 1,122.33 | 1,188.07 |
| PLUNC | 662.40 | 896.18 | 1,058.82 | 1,080.51 |
| Pref-1 | 859.47 | 1,358.95 | 1,536.54 | 1,644.12 |
| Progranulin | 653.74 | 1,963.25* | 2,328.68* | 1,521.58* |
| Prolactin | 880.05 | 1,536.04 | 1,752.35* | 1,937.09* |
| P-selectin | 1,210.84 | 1,728.37 | 2,182.05 | 2,064.46 |
| RAGE | 1,138.29 | 1,214.77 | 1,404.05 | 1,597.04 |
| RANK/ | 1,444.73 | 1,779.27 | 1,990.66 | 2,221.48 |
| TNFRSF11A | | | | |
| RANTES | 975.87 | 1,489.53 | 1,341.73 | 1,519.37 |
| RELM beta | 852.43 | 1,569.27 | 1,380.69 | 1,447.78 |
| RELT/ | 1,193.52 | 2,109.96 | 1,898.43 | 2,192.58 |
| TNFRSF19L | | | | |
| ROBO4 | 927.69 | 1,908.04* | 1,408.25 | 1,820.76 |
| S100 A8/A9 | 1,204.88 | 2,363.44 | 2,269.53 | 2,659.45* |
| S100A10 | 6,838.69 | 10,077.83 | 10,485.84 | 10,679.37 |
| SAA | 1,109.06 | 1,550.81 | 1,548.44 | 1,894.56 |
| SCF | 3,868.03 | 4,734.89 | 5,077.24 | 6,759.07 |
| SCF R/ | 1,331.57 | 1,372.95 | 1,607.01 | 1,790.92 |
| CD117 | | | | |
| SDF-1/ | 1,002.40 | 1,153.57 | 1,404.86 | 1,486.05 |
| CXCL12 | | | | |
| sFRP-1 | 835.65 | 993.15 | 1,428.34 | 1,157.91 |
| sFRP-3 | 1,607.69 | 1,452.67 | 1,932.14 | 2,041.60 |
| sFRP-4 | 18,001.81 | 24,248.20 | 32,713.49 | 41,545.55* |
| sgp130 | 2,145.30 | 2,375.82 | 2,949.22 | 3,282.83 |
| SIGIRR | 3,403.51 | 2,541.45 | 3,038.71 | 3,827.48 |
| Siglec-5/ | 1,778.23 | 1,727.81 | 1,953.84 | 2,069.61 |
| CD170 | | | | |
| Siglec-9 | 1,628.80 | 1,493.06 | 2,025.24 | 2,542.46 |
| SLPI | 1,126.92 | 1,139.11 | 1,708.26 | 2,184.82 |
| Smad 1 | 2,072.75 | 1,431.28 | 1,354.99 | 2,104.24 |
| Smad 4 | 2,805.26 | 2,835.98 | 8,232.29* | 4,904.66 |
| Smad 5 | 1,359.73 | 1,243.34 | 5,963.74* | 1,906.20 |
| Smad 7 | 791.26 | 1,267.67 | 2,347.11* | 1,278.30 |
| Smad 8 | 862.18 | 1,451.05 | 1,283.31 | 1,418.07 |
| Prdx | 533.01 | 1,162.32* | 1,306.85* | 1,280.77* |
| Soggy-1 | 966.13 | 2,179.15* | 1,993.02* | 2,347.68* |
| Sonic Hedgehog | 704.63 | 1,508.09* | 1,550.51* | 1,747.30* |
| (Shh N-terminal) | | | | |
| SPARC | 956.92 | 8,863.21* | 8,852.43* | 14,857.38* |
| Spinesin | 743.61 | 1,736.29* | 2,210.92* | 2,700.87* |
| TACI/ | 1,051.13 | 1,835.72 | 2,038.77 | 2,507.01* |
| TNFRSF13B | | | | |
| Tarc | 873.01 | 1,469.03 | 1,386.36 | 2,127.31* |
| TCCR/WSX-1 | 1,014.85 | 1,512.51 | 1,455.58 | 1,956.80 |
| TECK/CCL25 | 1,024.06 | 1,636.63 | 1,511.86 | 1,868.04 |
| TFPI | 1,264.98 | 2,263.32 | 2,041.19 | 2,294.98 |
| TGF-alpha | 727.91 | 1,742.60* | 1,428.26 | 1,881.89* |
| TGF-beta 1 | 876.26 | 1,795.74* | 1,418.00 | 1,906.18* |
| TGF-beta 2 | 1,190.27 | 1,874.23 | 1,888.48 | 2,330.21 |
| TGF-beta 3 | 1,098.77 | 1,771.08 | 1,947.87 | 2,354.60* |
| TGF-beta 5 | 5,353.09 | 7,576.21 | 9,248.68 | 7,894.59 |
| TGF-beta | 1,720.84 | 2,988.49 | 3,184.01 | 3,341.05 |
| RI/ALK-5 | | | | |
| TGF-beta RII | 1,030.55 | 1,407.11 | 1,687.88 | 1,817.73 |
| Grb2 | 943.39 | 1,110.77 | 1,390.05 | 1,438.76 |
| TGF-beta RIII | 1,486.41 | 1,385.01 | 1,861.39 | 1,824.49 |
| Thrombopoietin | 2,431.70 | 2,241.07 | 3,233.90 | 2,987.64 |
| (TPO) | | | | |
| TPX | 4,423.51 | 3,688.17 | 3,254.43 | 4,908.28 |
| Thrombo- | 64,279.03 | 65,678.75 | 49,684.26 | 82,706.21 |
| spondin-1 | | | | |
| Thrombo- | 5,093.76 | 5,089.23 | 4,487.73 | 9,647.94 |
| spondin-2 | | | | |
| Thrombo- | 1,697.02 | 1,385.86 | 1,678.72 | 2,814.18 |
| spondin-4 | | | | |
| Thymopoietin | 1,039.22 | 998.68 | 1,456.02 | 2,411.46* |
| Tie-1 | 1,588.20 | 1,547.54 | 2,148.41 | 2,450.29 |
| Tie-2 | 1,206.51 | 2,185.01 | 2,345.60 | 3,182.89* |
| TIMP-1 | 1,295.84 | 14,201.02* | 16,620.57* | 25,661.55* |
| TIMP-2 | 820.49 | 28,405.89* | 22,696.06* | 26,642.47* |
| TIMP-3 | 559.00 | 1,753.62* | 2,267.47* | 2,468.84* |
| TIMP-4 | 1,091.73 | 1,806.14 | 2,138.00 | 2,983.50* |
| TL1A/T | 1,014.31 | 1,508.55 | 1,601.68 | 2,125.42* |
| NFSF15 | | | | |
| TLR1 | 943.93 | 1,607.92 | 1,872.94 | 2,282.20* |
| TLR2 | 932.56 | 1,872.01* | 2,033.23* | 2,700.80* |
| TLR3 | 1,183.23 | 2,016.94 | 1,977.37 | 2,862.29* |
| TLR4 | 1,130.71 | 1,801.69 | 1,803.61 | 2,677.74* |
| TMEFF1/ | 1,668.32 | 2,141.06 | 2,194.53 | 3,544.15* |
| Tomoregulin-1 | | | | |
| TMEFF2 | 1,064.12 | 1,974.74 | 2,133.12* | 2,626.45* |
| TNF-alpha | 1,092.27 | 1,822.24 | 1,569.82 | 2,494.98* |
| TNF-beta | 1,312.62 | 2,217.41 | 2,084.36 | 3,590.22* |
| TNF RI/ | 1,078.20 | 1,586.44 | 1,452.81 | 2,172.68* |
| TNFRSF1A | | | | |
| TNF RII/ | 2,177.78 | 1,927.28 | 2,208.75 | 3,175.53 |
| TNFRSF1B | | | | |
| TRADD | 1,475.04 | 1,476.87 | 1,520.04 | 2,104.60 |
| TRAIL/ | 1,321.29 | 1,307.47 | 1,593.30 | 2,226.75 |
| TNFSF10 | | | | |
| TRAIL | 1,504.82 | 1,329.39 | 1,531.22 | 1,924.02 |
| R1/DR4/ | | | | |
| TNFRSF10A | | | | |
| TRAIL | 1,995.87 | 1,518.44 | 1,932.76 | 2,443.97 |
| R2/DR5/ | | | | |
| TNFRSF10B | | | | |
| TRAIL R3/ | 2,222.17 | 2,274.87 | 3,171.18 | 2,904.21 |
| TNFRSF10C | | | | |
| TRAIL R4/ | 2,354.28 | 1,965.74 | 2,118.98 | 2,338.33 |
| TNFRSF10D | | | | |
| TRANCE | 5,532.83 | 4,363.26 | 4,194.06 | 6,347.90 |
| TREM-1 | 1,413.87 | 1,333.65 | 1,923.92 | 2,503.24 |
| TROY/ | 1,098.23 | 1,546.46 | 1,980.55 | 2,656.45* |
| TNFRSF19 | | | | |
| TSG-6 | 1,122.59 | 1,787.06 | 2,297.39* | 2,782.84* |
| TSLP R | 1,051.67 | 1,285.14 | 1,500.27 | 1,813.06 |
| TWEAK/ | 1,177.27 | 1,341.84 | 1,620.97 | 2,050.17 |
| TNFSF12 | | | | |
| TWEAK R/ | 1,171.32 | 1,830.00 | 1,680.44 | 2,274.93 |
| TNFRSF12 | | | | |
| Ubiquitin + 1 | 1,439.85 | 1,723.28 | 1,994.16 | 2,801.97 |
| uPA | 1,427.94 | 1,843.28 | 2,078.28 | 3,230.23* |
| uPAR | 1,265.52 | 1,973.16 | 1,990.85 | 2,855.24* |
| Vasorin | 1,321.29 | 2,213.59 | 2,146.23 | 3,898.00* |
| VCAM-1 | 1,267.15 | 1,843.52 | 1,499.51 | 2,820.62* |
| (CD106) | | | | |
| VE-Cadherin | 1,219.50 | 1,974.43 | 1,530.16 | 3,071.04* |
| VEGF | 1,548.67 | 2,275.91 | 2,431.67 | 4,687.78* |
| VEGF R2 (KDR) | 1,195.68 | 1,733.34 | 1,367.12 | 3,178.56* |
| VEGF R3 | 1,259.57 | 1,733.65 | 1,522.55 | 2,852.67* |
| VEGF-B | 1,093.36 | 1,309.05 | 1,272.29 | 2,274.69* |
| VEGF-C | 1,454.47 | 1,460.05 | 1,724.70 | 2,508.73 |
| VEGF-D | 2,017.53 | 1,857.74 | 2,348.98 | 3,409.78 |
| VEGI/ | 1,150.20 | 961.24 | 1,234.62 | 1,727.08 |
| TNFSF15 | | | | |
| WIF-1 | 1,257.40 | 1,211.39 | 1,556.77 | 1,921.83 |
| WISP-1/CCN4 | 1,195.68 | 1,347.81 | 1,584.64 | 1,646.05 |
| XEDAR | 1,414.95 | 1,592.80 | 1,822.48 | 2,751.63* |
| Positive | 19,613.33 | 19,613.33 | 19,613.33 | 19,613.33 |
| Control | | | | |
| Neg | 1.16 | 153.65* | 411.23* | 130.50* |
| 11b-HSD1 | 1.16 | 9.43* | 1.03 | 69.25* |
| 2B4 | 1.16 | 36.20* | 9.28* | 73.02* |
| 4-1BB | 1.16 | 44.55* | 19.13* | 67.57* |
| A1BG | 1.16 | 114.05* | 42.87* | 115.12* |
| A2M | 211.47 | 1,600.89* | 1,136.95* | 2,328.91* |
| ABL1 | 477.41 | 502.67 | 359.45 | 791.51 |
| ACE | 78.80 | 193.80* | 68.06 | 270.68* |
| ACE-2 | 194.09 | 326.54 | 237.09 | 405.17* |
| ACK1 | 477.41 | 647.94 | 512.69 | 686.25 |
| ACPP | 313.45 | 481.05 | 486.23 | 820.72* |
| ACTH | 414.84 | 595.44 | 800.49 | 1,444.69* |
| ADAM-9 | 446.70 | 472.90 | 706.03 | 1,062.23* |
| ADAMTS-1 | 717.85 | 367.14 | 851.87 | 985.02 |
| ADAMTS-10 | 1,469.89 | 3,168.98* | 3,839.53* | 5,165.18* |
| ADAMTS-13 | 719.59 | 247.44* | 601.62 | 574.50 |
| ADAMTS-15 | 555.63 | 121.77* | 558.53 | 438.44 |
| ADAMTS-17 | 300.12 | 96.17* | 345.21 | 295.50 |
| ADAMTS-18 | 63.73 | 83.59 | 448.50* | 505.98* |
| ADAMTS-19 | 1.16 | 50.06* | 361.51* | 207.95* |
| ADAMTS-4 | 1.16 | 58.51* | 313.92* | 161.25* |
| ADAMTS-5 | 1.16 | 11.18* | 286.18* | 218.20* |
| ADAMTS-L2 | 1.16 | 69.63* | 559.99* | 726.19* |
| Adipsin | 1.16 | 1,233.42* | 5,756.54* | 1,943.35* |
| Afamin | 1,208.59 | 141.80* | 753.15 | 2,936.23* |
| AFP | 2,181.37 | 65.56* | 684.57* | 1,530.48 |
| ALBUMIN | 4,443.86 | 604.61* | 1,541.23* | 2,317.00 |
| Aldolase A | 1.16 | 110.96* | 150.78* | 104.91* |
| Aldolase B | 1.16 | 33.13* | 50.87* | 26.21* |
| Aldolase C | 1.16 | 141.93* | 81.12* | 79.57* |
| ALK | 1.16 | 105.42* | 66.77* | 96.93* |
| Alpha 1 AG | 1.16 | 162.29* | 170.43* | 284.78* |
| Alpha 1 | 1.16 | 306.58* | 234.13* | 591.28* |
| Microglobulin | | | | |
| Alpha | 280.42 | 381.04 | 263.45 | 493.19 |
| Lactalbumin | | | | |
| ALPP | 183.08 | 513.50* | 455.35* | 786.07* |
| AMICA | 315.18 | 451.98 | 536.48 | 908.83* |
| AMPKa1 | 177.87 | 268.69 | 323.94 | 447.34* |
| Amylin | 358.64 | 523.14 | 649.22 | 836.06* |
| ANGPTL3 | 405.57 | 451.96 | 675.38 | 616.26 |
| ANGPTL4 | 541.14 | 344.80 | 784.43 | 884.98 |
| Annexin A7 | 603.14 | 264.54* | 743.79 | 753.06 |
| APC | 732.92 | 335.35* | 863.68 | 954.10 |
| APCS | 460.61 | 243.76 | 577.74 | 592.61 |
| Apelin | 651.80 | 383.81 | 700.36 | 730.64 |
| Apex1 | 823.88 | 304.48* | 516.56 | 508.56 |
| APN | 130.94 | 103.98 | 331.28* | 281.25* |
| ApoA1 | 145.42 | 91.21 | 347.19* | 317.18* |
| ApoA2 | 176.13 | 92.10* | 327.31 | 313.48 |
| ApoA4 | 175.55 | 115.46 | 507.64* | 554.46* |
| ApoB | 1.16 | 22.98* | 389.11* | 401.33* |
| ApoB100 | 303.60 | 27.93* | 401.06 | 543.36 |
| ApoC1 | 2,526.11 | 43.49* | 517.26* | 764.06* |
| ApoC2 | 6,480.97 | 1,667.31* | 3,446.03 | 3,573.17 |
| ApoC3 | 98,174.53 | 99,021.84 | 118,299.42 | 145,523.00 |
| ApoD | 1.16 | 270.70* | 422.17* | 411.17* |
| ApoE | 1.16 | 98.24* | 439.38* | 15.84* |
| ApoE3 | 1.16 | 119.91* | 195.12* | 22.22* |
| ApoH | 1.16 | 185.87* | 170.33* | 65.28* |
| ApoM | 1.16 | 178.23* | 219.26* | 187.02* |
| APP | 1.16 | 2,545.55* | 2,551.79* | 2,560.93* |
| ASPH | 1.16 | 204.00* | 204.89* | 174.64* |
| Attractin | 1.16 | 127.47* | 150.90* | 118.11* |
| B3GNT1 | 1.16 | 173.58* | 221.68* | 198.86* |
| BAF57 | 1.16 | 198.59* | 218.13* | 216.41* |
| BAFF | 1.16 | 201.77* | 264.89* | 252.07* |
| BAI-1 | 254.93 | 324.81 | 445.54 | 481.31 |
| BCAM | 325.61 | 498.10 | 671.55* | 627.02 |
| Beta 2M | 412.52 | 2,635.25* | 3,669.88* | 3,451.24* |
| Beta | 1,734.09 | 1,059.32 | 1,511.85 | 2,061.92 |
| Defensin 4 | | | | |
| Beta IG-H3 | 643.69 | 7,717.55* | 7,511.18* | 18,306.78* |
| Biglycan | 554.47 | 624.68 | 819.28 | 1,006.07 |
| BLAME | 695.84 | 383.35 | 677.00 | 644.94 |
| BMP-9 | 600.82 | 258.65* | 527.62 | 578.95 |
| BMX | 432.80 | 247.72 | 588.89 | 520.72 |
| BNIP2 | 854.01 | 708.38 | 1,044.63 | 722.72 |
| BNP | 108.34 | 229.07* | 393.92* | 406.62* |
| Btk | 230.01 | 342.75 | 617.12* | 667.78* |
| C2 | 151.80 | 175.42 | 412.82* | 314.57* |
| C3a | 301.28 | 69.16* | 333.27 | 259.56 |
| C5/C5a | 1.16 | 48.23* | 291.43* | 211.11* |
| C7 | 527.24 | 140.86* | 447.49 | 755.93 |
| C8B | 2,329.69 | 18.40* | 329.57* | 450.97* |
| C9 | 3,010.47 | 359.91* | 768.15* | 1,482.85* |
| CA9 | 2,275.23 | 2,029.32 | 2,016.56 | 4,700.01* |
| CA15-3 | 1.16 | 255.70* | 223.13* | 369.19* |
| CA19-9 | 1.16 | 301.47* | 335.34* | 153.30* |
| CA125 | 1.16 | 313.04* | 298.27* | 166.63* |
| Cadherin-13 | 1.16 | 314.86* | 458.31* | 413.20* |
| Calbindin | 2,618.81 | 2,167.38 | 2,064.99 | 3,732.71 |
| Calbindin D | 190.04 | 266.92 | 349.99 | 487.43* |
| Calcitonin | 191.78 | 230.39 | 223.17 | 353.91 |
| Calreticulin | 39.98 | 175.45* | 256.30* | 252.87* |
| Calsyntenin-1 | 1.16 | 137.85* | 305.11* | 182.14* |
| CART | 17.96 | 199.66* | 269.76* | 316.66* |
| Caspase-3 | 263.62 | 298.03 | 374.00 | 508.82 |
| Caspase-8 | 338.94 | 427.22 | 471.59 | 1,154.52* |
| Cathepsin B | 382.39 | 392.09 | 570.98 | 1,830.89* |
| Cathepsin D | 628.05 | 320.66 | 643.39 | 2,269.65* |
| Cathepsin L | 914.84 | 427.39* | 818.41 | 1,754.78 |
| Cathepsin S | 837.21 | 403.24* | 807.82 | 1,271.07 |
| CBP | 1,471.05 | 1,130.35 | 1,914.18 | 2,290.66 |
| CCK | 2,319.27 | 877.40* | 1,551.89 | 2,288.94 |
| CD23 | 964.67 | 200.71* | 592.20 | 713.19 |
| CD24 | 392.24 | 124.37* | 479.67 | 517.42 |
| CD36 | 355.74 | 72.25* | 452.72 | 422.25 |
| CD38 | 509.86 | 109.68* | 420.48 | 423.82 |
| CD44 | 336.04 | 29.20* | 269.98 | 315.01 |
| CD45 | 481.47 | 97.96* | 352.40 | 375.07 |
| CD46 | 390.50 | 91.75* | 328.05 | 335.17 |
| CD47 | 366.17 | 69.10* | 454.75 | 1,125.47* |
| CD55 | 343.57 | 45.41* | 339.31 | 392.46 |
| CD59 | 989.58 | 31.08* | 291.57* | 391.36* |
| CD61 | 751.46 | 108.98* | 510.44 | 820.02 |
| CD71 | 734.66 | 1,336.56 | 524.85 | 957.47 |
| CD74 | 1.16 | 478.12* | 577.33* | 721.31* |
| CD79 alpha | 85.75 | 386.11* | 594.13* | 603.35* |
| CD90 | 1.16 | 193.34* | 326.88* | 245.74* |
| CD97 | 161.07 | 169.18 | 402.60* | 337.07* |
| CD200 | 171.50 | 152.30 | 364.80* | 382.47* |
| CEA | 48.09 | 107.86* | 271.54* | 324.26* |
| CEACAM-1 | 1.16 | 118.58* | 272.14* | 350.90* |
| Ceruloplasmin | 1.16 | 62.42* | 210.45* | 231.17* |
| CFHR2 | 1.16 | 118.91* | 248.80* | 305.94* |
| Chemerin | 1.16 | 211.16* | 499.05* | 560.39* |
| CHI3L1 | 97.34 | 386.78* | 668.44* | 1,240.82* |
| Chromogranin A | 296.64 | 309.79 | 626.30* | 1,196.58* |
| Chymase | 474.51 | 259.06 | 553.40 | 1,080.93* |
| cIAP-2 | 652.96 | 278.39* | 688.69 | 1,306.68* |
| Ck beta 8-1 | 877.18 | 236.89* | 705.17 | 1,245.08 |
| CK-MB | 784.48 | 302.21* | 722.15 | 962.44 |
| Claudin-3 | 747.40 | 187.58* | 757.04 | 700.43 |
| Claudin-4 | 766.52 | 190.35* | 638.20 | 675.76 |
| CLEC3B | 443.81 | 168.00* | 645.21 | 824.09 |
| Clusterin | 447.28 | 35.90* | 695.19 | 641.26 |
| CNDP1 | 801.29 | 128.46* | 768.47 | 1,031.40 |
| COCO | 115.88 | 42.75* | 434.58* | 442.03* |
| Complement | 1.16 | 29.70* | 358.92* | 357.02* |
| factor H | | | | |
| Contactin-1 | 924.11 | 970.29 | 2,078.23* | 1,491.32 |
| Contactin-2 | 124.57 | 389.77* | 323.82* | 292.41* |
| Corticosteroid- | 346.47 | 258.77 | 1,509.12* | 1,007.06* |
| binding globulin | | | | |
| COX-2 | 125.73 | 106.79 | 367.71* | 509.07* |
| C-peptide | 566.64 | 253.52* | 783.19 | 1,310.38* |
| CPN2 | 974.52 | 436.21 | 1,540.54 | 1,731.99 |
| Creatinine | 24,459.77 | 27,733.72 | 36,863.11 | 42,606.45 |
| CRP | 1.16 | 274.28* | 622.99* | 499.57* |
| CRTAM | 56.78 | 126.63* | 402.34* | 252.36* |
| CSH1 | 315.76 | 163.67 | 601.50 | 379.19 |
| cTnT | 321.56 | 76.83* | 472.14 | 362.64 |
| CutA | 89.22 | 13.30* | 291.89* | 237.14* |
| Cyclin D1 | 52.72 | 19.48* | 338.28* | 281.74* |
| Cystatin A | 31.87 | 41.28 | 335.28* | 382.80* |
| Cystatin B | 143.11 | 168.01 | 399.29* | 434.29* |
| Cystatin C | 13.33 | 156.76* | 371.89* | 435.83* |
| Cytochrome C | 1.16 | 147.29* | 447.82* | 533.46* |
| Cytokeratin 8 | 1.74 | 186.03* | 461.25* | 570.57* |
| Cytokeratin18 | 308.81 | 247.58 | 646.00* | 687.05* |
| Cytokeratin19 | 286.21 | 198.38 | 603.87* | 553.21 |
| DBI | 440.33 | 173.25* | 599.73 | 482.00 |
| DCBLD2 | 570.11 | 149.49* | 655.67 | 591.06 |
| D-Dimer | 954.24 | 1,388.15 | 1,984.01* | 2,992.84* |
| DEFA1/3 | 742.77 | 110.47* | 630.23 | 630.80 |
| Defensin | 410.78 | 97.17* | 569.32 | 583.18 |
| Desmin | 646.01 | 265.86* | 914.42 | 1,195.33 |
| DLL1 | 713.22 | 152.91* | 800.76 | 836.42 |
| DLL4 | 464.66 | 103.12* | 743.53 | 626.97 |
| DMP-1 | 504.64 | 364.45 | 1,205.64* | 1,160.89* |
| DPPIV | 183.66 | 150.61 | 708.51* | 646.68* |
| E-Cadherin | 413.68 | 119.15* | 470.11 | 536.61 |
| Endorphin Beta | 204.52 | 86.27* | 380.05 | 288.02 |
| Endothelin | 195.25 | 125.60 | 406.49* | 304.60 |
| Receptor A | | | | |
| Enolase 2 | 200.47 | 134.83 | 474.45* | 402.37* |
| ENPP2 | 285.06 | 126.10 | 607.26* | 727.38* |
| EpCAM | 438.01 | 81.50* | 559.59 | 919.72* |
| EphA1 | 1,309.98 | 450.72* | 1,095.72 | 1,924.97 |
| EphA2 | 1.16 | 148.42* | 465.87* | 299.77* |
| EphA3 | 384.13 | 381.58 | 1,053.80* | 1,085.43* |
| EphA4 | 505.80 | 169.49* | 675.22 | 494.89 |
| EphA5 | 1,052.74 | 356.71* | 1,068.15 | 1,090.24 |
| EphA6 | 168.02 | 124.64 | 549.03* | 462.61* |
| EphA7 | 372.54 | 152.44* | 466.49 | 578.01 |
| EphA8 | 895.72 | 401.57* | 895.55 | 1,156.06 |
| EphB1 | 902.10 | 426.40* | 811.55 | 1,108.08 |
| EphB2 | 931.07 | 389.46* | 727.95 | 1,009.72 |
| EphB3 | 851.69 | 567.00 | 905.66 | 1,217.81 |
| EphB4 | 1.16 | 170.38* | 488.75* | 535.43* |
| EphB6 | 144.85 | 222.27 | 563.62* | 638.01* |
| ERRa | 544.62 | 256.42* | 905.78 | 1,132.77* |
| Erythro- | 628.63 | 325.89 | 727.64 | 731.78 |
| poietin R | | | | |
| ESAM | 672.66 | 260.47* | 677.34 | 639.52 |
| EV15L | 848.22 | 245.00* | 670.71 | 652.17 |
| EXTL2 | 738.13 | 375.10 | 934.56 | 963.63 |
| FABP1 | 669.77 | 212.31* | 810.51 | 1,015.16 |
| FABP2 | 367.91 | 162.11* | 565.40 | 748.39 |
| FABP3 | 250.29 | 140.27 | 501.71* | 614.11* |
| FABP4 | 529.56 | 828.56 | 1,852.88* | 2,616.67* |
| Factor XIII A | 18,130.02 | 48,560.06* | 59,351.02* | 79,043.79* |
| Factor XIII B | 690.04 | 1,868.35* | 2,488.59* | 3,152.99* |
| FAK | 505.80 | 102.03* | 489.58 | 511.57 |
| FAP | 1,625.17 | 129.78* | 490.19* | 468.82* |
| Fc RIIB/C | 820.98 | 124.61* | 444.18 | 507.94 |
| Fen 1 | 45.19 | 136.69* | 484.46* | 581.16* |
| FER | 396.88 | 185.63* | 685.08 | 1,029.11* |
| Ferritin | 626.89 | 58.44* | 517.85 | 768.93 |
| Fetuin A | 409.62 | 47.09* | 504.40 | 729.06 |
| Fetuin B | 1.16 | 200.35* | 768.38* | 569.76* |
| FGFR1 | 209.74 | 254.36 | 1,122.23* | 1,353.40* |
| FGFR1 alpha | 136.73 | 134.14 | 736.12* | 446.89* |
| FGFR2 | 694.68 | 508.85 | 1,270.68 | 1,459.78* |
| Fibrinogen | 530.13 | 9,202.76* | 13,333.01* | 21,925.77* |
| Fibrinopeptide A | 276.94 | 692.98* | 1,348.75* | 2,031.59* |
| Fibronectin | 550.41 | 18,570.43* | 28,116.82* | 41,847.45* |
| Ficolin-3 | 59.10 | 626.64* | 1,014.16* | 1,362.72* |
| FIH | 107.77 | 187.69 | 314.49* | 478.26* |
| FOLR1 | 266.52 | 371.75 | 648.03* | 804.23* |
| FOXN3 | 7,686.66 | 9,269.48 | 13,035.73 | 10,005.07 |
| FoxO1 | 578.22 | 619.85 | 947.14 | 1,115.03 |
| FoxP3 | 451.34 | 226.39* | 612.67 | 704.19 |
| FRK | 1,754.37 | 1,547.28 | 2,738.80 | 3,489.41 |
| FSH | 618.78 | 351.97 | 788.29 | 675.01 |
| Furin | 770.00 | 405.58 | 765.36 | 716.12 |
| Fyn | 771.74 | 312.23* | 672.14 | 621.95 |
| GADD45A | 711.48 | 407.14 | 742.24 | 889.34 |
| Galanin | 334.88 | 327.20 | 586.71 | 830.90* |
| Galectin-1 | 128.62 | 199.38 | 543.07* | 686.95* |
| Galectin-3BP | 243.92 | 330.51 | 732.15* | 947.58* |
| Galectin-7 | 513.91 | 1,083.84* | 1,486.76* | 2,699.46* |
| gamma-Thrombin | 47.51 | 233.70* | 586.83* | 667.71* |
| Gas1 | 320.98 | 185.42 | 593.10 | 547.42 |
| Gastrin | 312.29 | 67.87* | 489.67 | 412.76 |
| GATA-3 | 387.61 | 86.58* | 549.91 | 571.94 |
| GATA-4 | 2.32 | 54.24* | 469.00* | 453.90* |
| Gelsolin | 224.22 | 89.75* | 544.17* | 607.43* |
| Ghrelin | 615.88 | 94.58* | 748.09 | 878.58 |
| GLP-1 | 832.57 | 53.28* | 959.43 | 1,323.73 |
| GMNN | 1.16 | 245.25* | 695.85* | 527.85* |
| GPBB | 1.16 | 147.93* | 713.46* | 545.19* |
| GPI | 1.16 | 111.65* | 607.64* | 405.04* |
| GPR-39 | 284.48 | 232.12 | 581.32* | 506.37 |
| GPX1 | 281.58 | 592.73* | 829.28* | 926.51* |
| GPX3 | 245.66 | 296.84 | 540.85* | 943.51* |
| GRP | 298.38 | 938.00* | 1,120.75* | 1,747.49* |
| GRP75 | 1.16 | 346.33* | 406.21* | 766.37* |
| GRP78 | 190.62 | 299.02 | 366.03 | 698.84* |
| GSR | 237.55 | 375.76 | 468.69 | 780.06* |
| GST | 573.59 | 615.94 | 761.21 | 1,141.42 |
| HADHA | 345.31 | 319.92 | 485.54 | 691.57* |
| HAI-1 | 751.46 | 477.71 | 732.34 | 831.14 |
| HAI-2 | 596.18 | 411.40 | 630.86 | 735.68 |
| Haptoglobin | 798.97 | 520.79 | 835.12 | 882.58 |
| hCG alpha | 482.63 | 449.92 | 701.08 | 653.19 |
| hCGb | 599.08 | 404.72 | 705.61 | 698.29 |
| Hck | 661.07 | 696.80 | 970.65 | 1,456.76* |
| HE4 | 765.94 | 500.66 | 665.10 | 1,371.54 |
| Hemopexin | 99.65 | 246.91* | 522.14* | 645.26* |
| Hepcidin | 2,332.59 | 1,878.39 | 2,468.08 | 3,035.34 |
| HOXA10 | 418.89 | 250.02 | 733.49 | 969.12* |
| HSP10 | 233.49 | 201.92 | 573.54* | 612.09* |
| HSP20 | 81.11 | 168.70* | 524.19* | 631.09* |
| HSP27 | 265.94 | 954.61* | 1,434.63* | 1,441.15* |
| HSP32 | 378.92 | 121.52* | 622.81 | 924.98* |
| HSP40 | 214.95 | 104.23* | 557.77* | 696.00* |
| HSP60 | 213.79 | 83.41* | 553.85* | 708.74* |
| HSP70 | 2.90 | 16.42* | 567.39* | 716.37* |
| HSP90 | 145.42 | 20.96* | 615.34* | 588.50* |
| HSPA8 | 227.70 | 175.45 | 497.30* | 574.46* |
| HTRA2 | 466.40 | 304.41 | 741.02 | 1,110.21* |
| IBSP | 16,123.62 | 14,446.85 | 20,211.41 | 29,611.81 |
| IGF2BP1 | 912.53 | 598.70 | 993.81 | 1,447.17 |
| IGFBP-5 | 170.92 | 257.12 | 350.61* | 605.88* |
| IL-23p19 | 246.82 | 356.98 | 459.70 | 701.96* |
| IL-33 | 238.13 | 271.70 | 418.56 | 702.02* |
| IL-34 | 413.68 | 306.40 | 464.44 | 699.77 |
| IL36RN | 429.90 | 332.20 | 541.50 | 652.26 |
| INSL3 | 493.05 | 450.70 | 617.57 | 634.07 |
| INSRR | 684.25 | 428.69 | 572.56 | 642.92 |
| Integrin | 1,019.71 | 936.88 | 1,081.80 | 1,401.20 |
| alpha V | | | | |
| Itk | 1,013.34 | 928.22 | 1,737.00 | 1,736.11 |
| ITM2B | 390.50 | 445.20 | 1,130.71* | 897.85* |
| Kallikrein 2 | 176.71 | 346.85 | 656.22* | 892.67* |
| Kallikrein 5 | 269.99 | 357.07 | 950.22 | 1,267.60 |
| Kallikrein 6 | 112.98 | 200.30 | 625.79* | 669.91* |
| Kallikrein 7 | 178.45 | 314.16 | 663.73* | 899.42* |
| Kallikrein 8 | 1.16 | 228.78* | 520.51* | 526.08* |
| Kallikrein 10 | 23.18 | 244.84* | 551.24* | 672.79* |
| Kallikrein 11 | 1.16 | 169.94* | 448.90* | 517.98* |
| Kallikrein 14 | 1.16 | 218.18* | 482.29* | 582.76* |
| KCC3 | 1.16 | 140.90* | 534.97* | 476.73* |
| KCTD10 | 1.16 | 156.82* | 573.66* | 604.07* |
| KIF3B | 1.16 | 196.27* | 701.09* | 894.29* |
| KLF4 | 67.21 | 184.46* | 773.71* | 1,038.51* |
| LAG-3 | 440.91 | 87.13* | 475.77 | 523.69 |
| Layilin | 456.55 | 105.13* | 421.32 | 648.24 |
| LDL R | 969.89 | 436.83* | 792.51 | 1,051.27 |
| Legumain | 382.97 | 186.84* | 394.32 | 582.36 |
| LH | 148.32 | 154.21 | 275.06 | 499.92* |
| LIMPII | 324.45 | 126.23* | 293.81 | 655.25* |
| LIN41 | 333.14 | 195.59 | 286.60 | 640.24 |
| Livin | 402.67 | 208.20 | 365.33 | 532.68 |
| LOX-1 | 579.96 | 330.34 | 459.19 | 704.74 |
| LPS | 1,444.98 | 936.70 | 1,516.72 | 3,377.81* |
| LRG1 | 984.95 | 445.33* | 744.38 | 1,021.03 |
| LTF | 910.79 | 732.79 | 1,276.51 | 1,319.82 |
| LTK | 1,148.33 | 770.68 | 1,050.21 | 1,139.59 |
| Lumican | 386.45 | 224.88 | 532.11 | 665.56 |
| Lyn | 557.94 | 703.58 | 1,178.34* | 1,462.97* |
| LYRIC | 265.94 | 258.97 | 552.31 | 1,155.46* |
| LYVE-1 | 1.16 | 227.30* | 497.99* | 532.83* |
| LZTS1 | 101.97 | 221.95* | 548.54* | 757.35* |
| Mammaglobin A | 1.16 | 217.39* | 428.96* | 742.71* |
| Marapsin | 64.31 | 234.40* | 378.03* | 602.72* |
| MATK | 39.40 | 323.89* | 628.50* | 759.82* |
| MBL | 45.77 | 332.55* | 576.16* | 784.43* |
| MBL-2 | 151.80 | 421.26* | 753.06* | 1,394.77* |
| Mer | 236.39 | 217.85 | 623.56 | 680.69 |
| Mesothelin | 42.87 | 202.92* | 629.06* | 619.08* |
| MICB | 258.40 | 229.81 | 732.37* | 721.71* |
| Midkine | 337.20 | 115.15* | 434.42 | 486.19 |
| MINA | 680.19 | 173.09* | 590.88 | 743.97 |
| MSHa | 329.09 | 42.74* | 280.93 | 486.59 |
| MTUS1 | 669.19 | 116.72* | 381.78 | 993.23 |
| Myoglobin | 436.85 | 127.78* | 256.00 | 483.10 |
| NAIP | 346.47 | 103.11* | 217.91 | 469.72 |
| Nanog | 454.24 | 137.26* | 231.49 | 491.24 |
| NELL2 | 368.49 | 198.31 | 320.67 | 460.11 |
| NEP | 590.39 | 293.14 | 413.02 | 666.76 |
| Nesfatin | 1,358.65 | 772.73 | 1,075.13 | 1,637.44 |
| Nestin | 659.92 | 324.28* | 555.09 | 600.03 |
| NET1 | 705.69 | 330.06* | 493.78 | 577.33 |
| Netrin G2 | 455.97 | 227.86 | 534.65 | 527.96 |
| Netrin-4 | 433.38 | 156.80* | 452.15 | 487.89 |
| Neurokinin-A | 278.10 | 284.81 | 535.09 | 594.02 |
| Neuropeptide Y | 85.75 | 220.99* | 680.95* | 540.43* |
| NF1 | 1.16 | 175.96* | 445.36* | 457.17* |
| NM23-H1/H2 | 180.19 | 119.58 | 410.80* | 403.92* |
| Notch-1 | 1.16 | 205.31* | 429.90* | 455.91* |
| NPTX1 | 1.16 | 261.23* | 430.64* | 465.00* |
| NPTXR | 1.16 | 215.70* | 355.49* | 514.51* |
| NR3C3 | 1.16 | 757.02* | 402.10* | 431.06* |
| Ntn1 | 814.03 | 632.94 | 1,663.06* | 1,882.51* |
| OCT3/4 | 61.99 | 297.65* | 514.87* | 566.50* |
| Omentin | 2,597.95 | 2,435.18 | 3,536.67 | 4,293.12 |
| Osteocalcin | 257.82 | 201.88 | 610.42* | 435.75 |
| Osteopontin | 336.62 | 364.06 | 868.17* | 381.26 |
| OX40 | 438.01 | 288.30 | 776.16 | 328.95 |
| p21 | 420.05 | 240.09 | 700.30* | 408.30 |
| p27 | 502.90 | 190.55* | 542.61 | 342.82 |
| p53 | 347.05 | 130.49* | 455.78 | 338.62 |
| PAI-1 | 20.86 | 701.04* | 1,662.47* | 2,454.59* |
| PAK7 | 198.15 | 197.43 | 336.99 | 480.83* |
| Pancreastatin | 545.20 | 173.38* | 463.08 | 502.72 |
| Pancreatic | 329.67 | 57.79* | 299.19 | 419.26 |
| Polypeptide | | | | |
| Pappalysin-1 | 373.12 | 119.07* | 270.21 | 445.54 |
| PARK7 | 130.94 | 145.20 | 335.55* | 439.50* |
| P-Cadherin | 466.40 | 271.86 | 489.41 | 580.17 |
| PCAF | 298.38 | 246.49 | 445.97 | 482.03 |
| PD-1 | 384.71 | 325.49 | 543.54 | 617.89 |
| PDX-1 | 512.75 | 277.83 | 481.55 | 433.81 |
| PEDF | 380.65 | 214.19 | 542.23 | 550.61 |
| PEPSINOGEN I | 256.09 | 179.04 | 465.05 | 449.14 |
| PEPSINOGEN II | 112.98 | 157.87 | 515.22* | 548.96* |
| PGRP-S | 23.75 | 286.05* | 626.66* | 877.90* |
| PI 16 | 1.16 | 154.88* | 632.80* | 830.47* |
| PI 3Kinase | 37.66 | 129.52* | 536.89* | 590.47* |
| p85 beta | | | | |
| PIM2 | 1.16 | 50.27* | 360.55* | 363.27* |
| PKM2 | 1.16 | 146.93* | 398.24* | 551.75* |
| Plasminogen | 1.16 | 1,137.55* | 3,677.66* | 3,358.75* |
| Podocalyxin | 1.16 | 189.31* | 509.40* | 505.68* |
| POMC | 223.06 | 407.33 | 702.04* | 1,022.91* |
| PON1 | 1.16 | 304.75* | 492.62* | 452.95* |
| PON2 | 1.16 | 110.61* | 499.41* | 690.37* |
| PPARg2 | 53.30 | 214.39* | 683.36* | 575.00* |
| PPP2R5C | 133.26 | 295.86* | 929.31* | 915.39* |
| Presenilin 1 | 444.97 | 355.83 | 974.47* | 387.80 |
| Presenilin 2 | 290.27 | 294.29 | 766.72* | 277.90 |
| Pro-BDNF | 445.54 | 261.57 | 550.60 | 295.81 |
| Procalcitonin | 187.14 | 195.88 | 600.62* | 377.33* |
| Pro-Cathepsin B | 68.95 | 299.96* | 745.63* | 529.31* |
| Progesterone | 1.16 | 68.74* | 446.96* | 353.70* |
| pro-Glucagon | 314.60 | 131.63* | 689.57* | 1,104.88* |
| Prohibitin | 1.16 | 84.54* | 303.03* | 379.16* |
| Pro-MMP-7 | 1.16 | 110.58* | 328.87* | 359.80* |
| Pro-MMP-9 | 79.95 | 98.30 | 298.81* | 393.38* |
| Pro-MMP-13 | 249.71 | 171.72 | 423.65 | 480.26 |
| ProSAAS | 121.09 | 306.45* | 617.31* | 523.80* |
| Prostasin | 186.56 | 289.62 | 664.56* | 618.30* |
| Protein p65 | 170.92 | 215.02 | 511.90* | 404.87* |
| PSA-Free | 144.85 | 223.69 | 524.95* | 501.94* |
| PSA-total | 220.74 | 179.48 | 594.41* | 545.79* |
| PSP | 1.16 | 125.16* | 455.47* | 589.44* |
| PTH | 1.16 | 533.71* | 845.09* | 2,143.04* |
| PTHLP | 7,014.00 | 30,003.94* | 25,623.63* | 43,869.68* |
| PTN | 1.16 | 815.10* | 1,204.22* | 1,688.76* |
| PTPRD | 314.60 | 323.72 | 857.87* | 1,229.51* |
| PYK2 | 1.16 | 122.90* | 485.82* | 801.01* |
| PYY | 165.12 | 386.93* | 1,005.10* | 1,993.06* |
| Ras | 66.63 | 3,697.36* | 8,124.49* | 14,353.17* |
| RBP4 | 1.16 | 195.55* | 747.93* | 748.67* |
| RECK | 1.16 | 240.27* | 647.48* | 692.97* |
| RELM alpha | 181.93 | 303.27 | 858.80* | 672.35* |
| Resistin | 1.16 | 125.17* | 600.75* | 581.38* |
| RET | 1.16 | 232.98* | 735.07* | 651.36* |
| RIP1 | 1.16 | 115.00* | 672.18* | 355.42* |
| ROCK1 | 1,150.07 | 447.56* | 1,261.36 | 1,108.93 |
| ROCK2 | 625.15 | 358.61 | 935.14 | 669.09 |
| ROR1 | 311.71 | 356.60 | 682.71* | 378.45 |
| ROR2 | 51.56 | 250.44* | 510.09* | 265.49* |
| ROS | 34.18 | 272.58* | 805.13* | 692.06* |
| RYK | 670.34 | 349.57 | 854.57 | 910.82 |
| S100A4 | 25.49 | 106.27* | 548.25* | 557.05* |
| S100A6 | 1.16 | 167.09* | 655.29* | 755.59* |
| S100A8 | 1.16 | 63.15* | 530.90* | 625.94* |
| S-100b | 19.70 | 66.68* | 358.95* | 481.14* |
| SART1 | 60.26 | 91.06 | 405.98* | 400.59* |
| SART3 | 67.79 | 91.22 | 522.90* | 445.85* |
| SCG3 | 35.92 | 135.93* | 542.17* | 474.98* |
| Selenoprotein P | 1,366.18 | 1,031.64 | 1,830.32 | 2,211.78 |
| SEMA3A | 234.65 | 170.55 | 791.12* | 529.39* |
| Serotonin | 144.27 | 76.71 | 658.75* | 589.15* |
| Serping 1 | 506.96 | 467.61 | 1,220.29* | 1,300.01* |
| Serpin A1 | 210.90 | 190.35 | 726.38* | 937.22* |
| Serpin A3 | 310.55 | 932.66* | 1,181.20* | 1,906.23* |
| Serpin A4 | 1.16 | 119.99* | 622.57* | 642.78* |
| Serpin A5 | 1.16 | 55.56* | 559.85* | 669.43* |
| Serpin A8 | 1,717.87 | 1,718.74 | 2,992.20 | 4,339.85* |
| Serpin A9 | 1.16 | 19.85* | 496.21* | 526.73* |
| Serpin A12 | 1.16 | 125.63* | 589.49* | 761.49* |
| Serpin B5 | 1.16 | 66.45* | 466.03* | 420.79* |
| Serpin D1 | 1.16 | 11.46* | 434.52* | 239.48* |
| Serpin I1 | 1.16 | 6.19* | 510.88* | 365.53* |
| SERTAD2 | 1.16 | 85.84* | 597.65* | 580.78* |
| SHBG | 1.16 | 128.57* | 859.98* | 562.89* |
| SMAC | 1.16 | 20.76* | 700.13* | 268.20* |
| SNCG | 7,622.35 | 4,667.80 | 5,455.71 | 9,088.43 |
| Somatotropin | 1,280.43 | 430.37* | 1,081.35 | 500.02* |
| SOST | 725.39 | 336.02* | 801.78 | 438.27 |
| SOX17 | 296.06 | 224.82 | 610.50* | 390.03 |
| SOX2 | 307.07 | 179.94 | 660.12* | 472.10 |
| SPARCL1 | 1,124.00 | 602.01 | 1,391.83 | 1,676.50 |
| SPINK1 | 1.16 | 60.57* | 476.02* | 478.31* |
| SRMS | 381.23 | 560.22 | 1,276.04* | 1,579.85* |
| SSEA-1 | 1.16 | 125.19* | 629.99* | 603.41* |
| SSEA-4 | 97.34 | 347.22* | 543.72* | 617.26* |
| SSTR2 | 270.57 | 383.46 | 807.43* | 918.23* |
| SSTR5 | 1.16 | 238.95* | 761.82* | 622.90* |
| Survivin | 191.20 | 238.95 | 833.72* | 588.45* |
| SYK | 425.85 | 250.15 | 984.88* | 684.07 |
| Syndecan-1 | 234.07 | 283.81 | 890.53* | 650.29* |
| Syndecan-3 | 39.98 | 90.24* | 807.73* | 969.06* |
| TACE | 161.65 | 99.81 | 819.77* | 629.18* |
| TAF4 | 1.16 | 32.94* | 647.13* | 518.04* |
| Tec | 274.05 | 234.99 | 1,106.33* | 1,179.67* |
| TFF1 | 553.31 | 427.03 | 1,170.02* | 1,457.40* |
| TFF3 | 1.16 | 117.74* | 548.67* | 788.19* |
| Thrombin | 1.16 | 131.45* | 560.74* | 492.24* |
| Thrombomodulin | 1.16 | 108.75* | 488.74* | 359.09* |
| Thymidine | 1.16 | 84.36* | 529.55* | 418.31* |
| Kinase-1 | | | | |
| Thyroglobulin | 1.16 | 629.61* | 570.80* | 376.26* |
| TIM-1 | 1.16 | 134.86* | 564.46* | 450.91* |
| TNK1 | 847.64 | 1,215.16 | 1,890.56* | 2,189.62* |
| TOPORS | 1.16 | 156.34* | 667.07* | 423.96* |
| TPA | 1.16 | 158.32* | 707.80* | 364.07* |
| TPM1 | 1.16 | 167.65* | 783.88* | 407.91* |
| TRA-1-60 | 272.31 | 195.13 | 782.50* | 406.75 |
| TRA-1-81 | 146.58 | 188.10 | 666.38* | 409.63 |
| Transferrin | 292.59 | 258.05 | 701.47* | 504.41 |
| Trappin-2 | 48.67 | 120.79* | 542.44* | 524.63* |
| TRKB | 250.87 | 275.42 | 765.39* | 681.24* |
| Troponin I | 189.46 | 238.62 | 635.42* | 518.48* |
| Troponin C | 441.49 | 377.57 | 884.41* | 904.31* |
| TRPC1 | 3,001.20 | 2,611.70 | 3,843.63 | 3,197.62 |
| TRPC6 | 216.69 | 268.23 | 885.02* | 552.92* |
| TRPM7 | 264.20 | 322.11 | 954.04* | 1,038.30* |
| Trypsin 1 | 377.18 | 324.59 | 1,028.25* | 634.40 |
| TSH | 180.19 | 250.15 | 1,077.27* | 586.90* |
| TSLP | 127.46 | 294.62* | 1,419.29* | 678.46* |
| TXK | 1,507.55 | 1,977.02 | 3,083.03* | 3,098.88* |
| Tyk2 | 58.52 | 176.40* | 951.65* | 589.09* |
| TYRO10 | 45.19 | 226.37* | 849.45* | 756.23* |
| Uromodulin | 1,234.08 | 87.29* | 711.55 | 428.77* |
| Vasopressin | 1.16 | 141.88* | 670.52* | 460.77* |
| VDUP-1 | 16.80 | 163.28* | 906.36* | 600.84* |
| VEGF R1 | 1.16 | 144.03* | 609.10* | 429.33* |
| VGF | 1.16 | 71.98* | 563.05* | 315.72* |
| VIP Receptor 2 | 1.16 | 127.08* | 609.75* | 265.00* |
| Visfatin | 1.16 | 175.82* | 825.67* | 463.03* |
| Vitamin D | 3,224.84 | 3,079.79 | 3,742.43 | 5,376.98 |
| Receptor | | | | |
| Vitamin D-BP | 88.07 | 201.02* | 629.39* | 212.06* |
| Vitamin | 1.16 | 188.38* | 488.99* | 279.33* |
| K-dependent | | | | |
| protein S | | | | |
| Vitronectin | 163.97 | 300.66 | 503.70* | 692.94* |
| VWF | 4,164.60 | 4,004.52 | 4,065.54 | 5,997.39 |
| Wilms Tumor 1 | 400.93 | 544.67 | 755.06 | 683.51 |
| XIAP | 461.19 | 534.02 | 620.92 | 645.07 |
| ZAG | 461.77 | 605.23 | 756.15 | 739.26 |
| ZAP70 | 482.63 | 665.08 | 799.14 | 620.49 |

Table 1 shows that a total of 661/1000 (66.1%) of the tested proteins demonstrated Biological Significance as described in the testing protocol (≧2 fold increase/decrease relative to control/comparator sample) in at least one of the averaged sample groups.

Table 2 is a listing of average production levels for each sample ethnic group (As=Asian, Af=African, Ca=Caucasian) that demonstrated Biological Significance across all three (3) groups. These shared proteins became the basis for the initial assessment that follows.

TABLE 2

| | | AS | AF | CA |
|---|---|---|---|---|
| | Control | Average | Average | Average |
| BCMA/TNFRSF17 | 1.08 | 67.49 | 208.10 | 194.35 |
| BAX | 90.68 | 552.02 | 357.12 | 759.17 |
| beta-NGF | 199.51 | 358.85 | 478.62 | 897.43 |
| CCL14/HCC-1/HCC-3 | 482.66 | 74.14 | 187.07 | 236.13 |
| CCR5 | 71.19 | 143.29 | 192.98 | 313.52 |
| CD30/TNFRSF8 | 111.26 | 272.19 | 446.20 | 489.25 |
| CV-2/ | 108.01 | 18.99 | 450.71 | 45.32 |
| Crossveinless-2 | | | | |
| CXCL16 | 44.67 | 293.17 | 389.68 | 380.72 |
| D6 | 216.83 | 435.25 | 652.24 | 453.43 |
| DAN | 686.77 | 1,597.31 | 2,464.56 | 2,888.04 |
| Decorin | 461.00 | 1,900.72 | 4,089.23 | 3,161.92 |
| Dkk-1 | 609.89 | 2,836.46 | 3,118.77 | 3,029.36 |
| Dkk-3 | 863.26 | 6,466.27 | 5,277.86 | 6,745.95 |
| EDA-A2 | 813.45 | 45,352.87 | 86,934.18 | 102,337.42 |
| EDAR | 578.49 | 1,715.75 | 2,752.31 | 3,558.44 |
| Eotaxin/CCL11 | 1,498.32 | 559.22 | 563.75 | 583.23 |
| FGF R5 | 674.85 | 1,554.38 | 2,334.15 | 2,276.60 |
| IGFBP-2 | 1,230.33 | 11,164.80 | 22,591.35 | 23,988.15 |
| IGFBP-3 | 1,211.38 | 4,239.29 | 5,899.29 | 7,880.22 |
| IGFBP-rp1/ | 11,808.74 | 76,254.45 | 90,204.78 | 94,029.81 |
| IGFBP-7 | | | | |
| IL-5 R alpha | 1,489.12 | 3,137.10 | 3,075.82 | 3,982.92 |
| IL-12 R beta 2 | 2,022.94 | 4,457.84 | 6,617.74 | 7,987.21 |
| Latent | 922.82 | 4,029.66 | 7,681.24 | 6,544.87 |
| TGF-beta bp1 | | | | |
| LFA-1 alpha | 611.51 | 1,354.79 | 1,250.51 | 1,524.15 |
| MIP-3 beta | 1,536.22 | 3,864.34 | 4,525.98 | 5,350.64 |
| MMP-1 | 974.25 | 46,620.53 | 53,622.33 | 38,420.38 |
| MMP-2 | 890.87 | 2,317.71 | 2,954.82 | 2,737.83 |
| MMP-3 | 871.92 | 6,219.51 | 7,061.18 | 8,496.31 |
| Nidogen-1 | 1,167.53 | 3,590.00 | 4,048.72 | 4,497.40 |
| NrCam | 1,193.52 | 2,442.48 | 3,271.70 | 3,210.07 |
| Osteoprotegerin/ | 974.25 | 2,120.02 | 2,149.40 | 3,157.01 |
| TNFRSF11B | | | | |
| Progranulin | 653.74 | 1,963.25 | 2,328.68 | 1,521.58 |
| Prdx | 533.01 | 1,162.32 | 1,306.85 | 1,280.77 |
| Soggy-1 | 966.13 | 2,179.15 | 1,993.02 | 2,347.68 |
| Sonic Hedgehog | 704.63 | 1,508.09 | 1,550.51 | 1,747.30 |
| (Shh N-terminal) | | | | |
| SPARC | 956.92 | 8,863.21 | 8,852.43 | 14,857.38 |
| Spinesin | 743.61 | 1,736.29 | 2,210.92 | 2,700.87 |
| TIMP-1 | 1,295.84 | 14,201.02 | 16,620.57 | 25,661.55 |
| TIMP-2 | 820.49 | 28,405.89 | 22,696.06 | 26,642.47 |
| TIMP-3 | 559.00 | 1,753.62 | 2,267.47 | 2,468.84 |
| TLR2 | 932.56 | 1,872.01 | 2,033.23 | 2,700.80 |
| 2B4 | 1.16 | 36.20 | 9.28 | 73.02 |
| 4-1BB | 1.16 | 44.55 | 19.13 | 67.57 |
| A1BG | 1.16 | 114.05 | 42.87 | 115.12 |
| A2M | 211.47 | 1,600.89 | 1,136.95 | 2,328.91 |
| ADAMTS-10 | 1,469.89 | 3,168.98 | 3,839.53 | 5,165.18 |
| ADAMTS-19 | 1.16 | 50.06 | 361.51 | 207.95 |
| ADAMTS-4 | 1.16 | 58.51 | 313.92 | 161.25 |
| ADAMTS-5 | 1.16 | 11.18 | 286.18 | 218.20 |
| ADAMTS-L2 | 1.16 | 69.63 | 559.99 | 726.19 |
| Adipsin | 1.16 | 1,233.42 | 5,756.54 | 1,943.35 |
| Aldolase A | 1.16 | 110.96 | 150.78 | 104.91 |
| Aldolase B | 1.16 | 33.13 | 50.87 | 26.21 |
| Aldolase C | 1.16 | 141.93 | 81.12 | 79.57 |
| ALK | 1.16 | 105.42 | 66.77 | 96.93 |
| Alpha 1 AG | 1.16 | 162.29 | 170.43 | 284.78 |
| Alpha 1 | 1.16 | 306.58 | 234.13 | 591.28 |
| Microglobulin | | | | |
| ALPP | 183.08 | 513.50 | 455.35 | 786.07 |
| ApoB | 1.16 | 22.98 | 389.11 | 401.33 |
| ApoD | 1.16 | 270.70 | 422.17 | 411.17 |
| ApoE | 1.16 | 98.24 | 439.38 | 15.84 |
| ApoE3 | 1.16 | 119.91 | 195.12 | 22.22 |
| ApoH | 1.16 | 185.87 | 170.33 | 65.28 |
| ApoM | 1.16 | 178.23 | 219.26 | 187.02 |
| APP | 1.16 | 2,545.55 | 2,551.79 | 2,560.93 |
| ASPH | 1.16 | 204.00 | 204.89 | 174.64 |
| Attractin | 1.16 | 127.47 | 150.90 | 118.11 |
| B3GNT1 | 1.16 | 173.58 | 221.68 | 198.86 |
| BAF57 | 1.16 | 198.59 | 218.13 | 216.41 |
| BAFF | 1.16 | 201.77 | 264.89 | 252.07 |
| Beta 2M | 412.52 | 2,635.25 | 3,669.88 | 3,451.24 |
| Beta IG-H3 | 643.69 | 7,717.55 | 7,511.18 | 18,306.78 |
| BNP | 108.34 | 229.07 | 393.92 | 406.62 |
| C5/C5a | 1.16 | 48.23 | 291.43 | 211.11 |
| C8B | 2,329.69 | 18.40 | 329.57 | 450.97 |
| C9 | 3,010.47 | 359.91 | 768.15 | 1,482.85 |
| CA15-3 | 1.16 | 255.70 | 223.13 | 369.19 |
| CA19-9 | 1.16 | 301.47 | 335.34 | 153.30 |
| CA125 | 1.16 | 313.04 | 298.27 | 166.63 |
| Cadherin-13 | 1.16 | 314.86 | 458.31 | 413.20 |
| Calreticulin | 39.98 | 175.45 | 256.30 | 252.87 |
| Calsyntenin-1 | 1.16 | 137.85 | 305.11 | 182.14 |
| CART | 17.96 | 199.66 | 269.76 | 316.66 |
| CD59 | 989.58 | 31.08 | 291.57 | 391.36 |
| CD74 | 1.16 | 478.12 | 577.33 | 721.31 |
| CD79 alpha | 85.75 | 386.11 | 594.13 | 603.35 |
| CD90 | 1.16 | 193.34 | 326.88 | 245.74 |
| CEA | 48.09 | 107.86 | 271.54 | 324.26 |
| CEACAM-1 | 1.16 | 118.58 | 272.14 | 350.90 |
| Ceruloplasmin | 1.16 | 62.42 | 210.45 | 231.17 |
| CFHR2 | 1.16 | 118.91 | 248.80 | 305.94 |
| Chemerin | 1.16 | 211.16 | 499.05 | 560.39 |
| CHI3L1 | 97.34 | 386.78 | 668.44 | 1,240.82 |
| COCO | 115.88 | 42.75 | 434.58 | 442.03 |
| Complement | 1.16 | 29.70 | 358.92 | 357.02 |
| factor H | | | | |
| Contactin-2 | 124.57 | 389.77 | 323.82 | 292.41 |
| CRP | 1.16 | 274.28 | 622.99 | 499.57 |
| CRTAM | 56.78 | 126.63 | 402.34 | 252.36 |
| CutA | 89.22 | 13.30 | 291.89 | 237.14 |
| Cyclin D1 | 52.72 | 19.48 | 338.28 | 281.74 |
| Cystatin C | 13.33 | 156.76 | 371.89 | 435.83 |
| Cytochrome C | 1.16 | 147.29 | 447.82 | 533.46 |
| Cytokeratin 8 | 1.74 | 186.03 | 461.25 | 570.57 |
| EphA2 | 1.16 | 148.42 | 465.87 | 299.77 |
| EphB4 | 1.16 | 170.38 | 488.75 | 535.43 |
| Factor XIII A | 18,130.02 | 48,560.06 | 59,351.02 | 79,043.79 |
| Factor XIII B | 690.04 | 1,868.35 | 2,488.59 | 3,152.99 |
| FAP | 1,625.17 | 129.78 | 490.19 | 468.82 |
| Fen 1 | 45.19 | 136.69 | 484.46 | 581.16 |
| Fetuin B | 1.16 | 200.35 | 768.38 | 569.76 |
| Fibrinogen | 530.13 | 9,202.76 | 13,333.01 | 21,925.77 |
| Fibrinopeptide A | 276.94 | 692.98 | 1,348.75 | 2,031.59 |
| Fibronectin | 550.41 | 18,570.43 | 28,116.82 | 41,847.45 |
| Ficolin-3 | 59.10 | 626.64 | 1,014.16 | 1,362.72 |
| Galectin-7 | 513.91 | 1,083.84 | 1,486.76 | 2,699.46 |
| gamma-Thrombin | 47.51 | 233.70 | 586.83 | 667.71 |
| GATA-4 | 2.32 | 54.24 | 469.00 | 453.90 |
| Gelsolin | 224.22 | 89.75 | 544.17 | 607.43 |
| GMNN | 1.16 | 245.25 | 695.85 | 527.85 |
| GPBB | 1.16 | 147.93 | 713.46 | 545.19 |
| GPI | 1.16 | 111.65 | 607.64 | 405.04 |
| GPX1 | 281.58 | 592.73 | 829.28 | 926.51 |
| GRP | 298.38 | 938.00 | 1,120.75 | 1,747.49 |
| GRP75 | 1.16 | 346.33 | 406.21 | 766.37 |
| Hemopexin | 99.65 | 246.91 | 522.14 | 645.26 |
| HSP20 | 81.11 | 168.70 | 524.19 | 631.09 |
| HSP27 | 265.94 | 954.61 | 1,434.63 | 1,441.15 |
| HSP40 | 214.95 | 104.23 | 557.77 | 696.00 |
| HSP60 | 213.79 | 83.41 | 553.85 | 708.74 |
| HSP70 | 2.90 | 16.42 | 567.39 | 716.37 |
| HSP90 | 145.42 | 20.96 | 615.34 | 588.50 |
| Kallikrein 8 | 1.16 | 228.78 | 520.51 | 526.08 |
| Kallikrein 10 | 23.18 | 244.84 | 551.24 | 672.79 |
| Kallikrein 11 | 1.16 | 169.94 | 448.90 | 517.98 |
| Kallikrein 14 | 1.16 | 218.18 | 482.29 | 582.76 |
| KCC3 | 1.16 | 140.90 | 534.97 | 476.73 |
| KCTD10 | 1.16 | 156.82 | 573.66 | 604.07 |
| KIF3B | 1.16 | 196.27 | 701.09 | 894.29 |
| KLF4 | 67.21 | 184.46 | 773.71 | 1,038.51 |
| LYVE-1 | 1.16 | 227.30 | 497.99 | 532.83 |
| LZTS1 | 101.97 | 221.95 | 548.54 | 757.35 |
| Mammaglobin A | 1.16 | 217.39 | 428.96 | 742.71 |
| Marapsin | 64.31 | 234.40 | 378.03 | 602.72 |
| MATK | 39.40 | 323.89 | 628.50 | 759.82 |
| MBL | 45.77 | 332.55 | 576.16 | 784.43 |
| MBL-2 | 151.80 | 421.26 | 753.06 | 1,394.77 |
| Mesothelin | 42.87 | 202.92 | 629.06 | 619.08 |
| Neuropeptide Y | 85.75 | 220.99 | 680.95 | 540.43 |
| NF1 | 1.16 | 175.96 | 445.36 | 457.17 |
| Notch-1 | 1.16 | 205.31 | 429.90 | 455.91 |
| NPTX1 | 1.16 | 261.23 | 430.64 | 465.00 |
| NPTXR | 1.16 | 215.70 | 355.49 | 514.51 |
| NR3C3 | 1.16 | 757.02 | 402.10 | 431.06 |
| OCT3/4 | 61.99 | 297.65 | 514.87 | 566.50 |
| PAI-1 | 20.86 | 701.04 | 1,662.47 | 2,454.59 |
| PGRP-S | 23.75 | 286.05 | 626.66 | 877.90 |
| PI 16 | 1.16 | 154.88 | 632.80 | 830.47 |
| PI 3Kinase | 37.66 | 129.52 | 536.89 | 590.47 |
| p85 beta | | | | |
| PIM2 | 1.16 | 50.27 | 360.55 | 363.27 |
| PKM2 | 1.16 | 146.93 | 398.24 | 551.75 |
| Plasminogen | 1.16 | 1,137.55 | 3,677.66 | 3,358.75 |
| Podocalyxin | 1.16 | 189.31 | 509.40 | 505.68 |
| PON1 | 1.16 | 304.75 | 492.62 | 452.95 |
| PON2 | 1.16 | 110.61 | 499.41 | 690.37 |
| PPARg2 | 53.30 | 214.39 | 683.36 | 575.00 |
| PPP2R5C | 133.26 | 295.86 | 929.31 | 915.39 |
| Pro-Cathepsin B | 68.95 | 299.96 | 745.63 | 529.31 |
| Progesterone | 1.16 | 68.74 | 446.96 | 353.70 |
| pro-Glucagon | 314.60 | 131.63 | 689.57 | 1,104.88 |
| Prohibitin | 1.16 | 84.54 | 303.03 | 379.16 |
| Pro-MMP-7 | 1.16 | 110.58 | 328.87 | 359.80 |
| ProSAAS | 121.09 | 306.45 | 617.31 | 523.80 |
| PSP | 1.16 | 125.16 | 455.47 | 589.44 |
| PTH | 1.16 | 533.71 | 845.09 | 2,143.04 |
| PTHLP | 7,014.00 | 30,003.94 | 25,623.63 | 43,869.68 |
| PTN | 1.16 | 815.10 | 1,204.22 | 1,688.76 |
| PYK2 | 1.16 | 122.90 | 485.82 | 801.01 |
| PYY | 165.12 | 386.93 | 1,005.10 | 1,993.06 |
| Ras | 66.63 | 3,697.36 | 8,124.49 | 14,353.17 |
| RBP4 | 1.16 | 195.55 | 747.93 | 748.67 |
| RECK | 1.16 | 240.27 | 647.48 | 692.97 |
| Resistin | 1.16 | 125.17 | 600.75 | 581.38 |
| RET | 1.16 | 232.98 | 735.07 | 651.36 |
| RIP1 | 1.16 | 115.00 | 672.18 | 355.42 |
| ROR2 | 51.56 | 250.44 | 510.09 | 265.49 |
| ROS | 34.18 | 272.58 | 805.13 | 692.06 |
| S100A4 | 25.49 | 106.27 | 548.25 | 557.05 |
| S100A6 | 1.16 | 167.09 | 655.29 | 755.59 |
| S100A8 | 1.16 | 63.15 | 530.90 | 625.94 |
| S-100b | 19.70 | 66.68 | 358.95 | 481.14 |
| SCG3 | 35.92 | 135.93 | 542.17 | 474.98 |
| Serpin A3 | 310.55 | 932.66 | 1,181.20 | 1,906.23 |
| Serpin A4 | 1.16 | 119.99 | 622.57 | 642.78 |
| Serpin A5 | 1.16 | 55.56 | 559.85 | 669.43 |
| Serpin A9 | 1.16 | 19.85 | 496.21 | 526.73 |
| Serpin A12 | 1.16 | 125.63 | 589.49 | 761.49 |
| Serpin B5 | 1.16 | 66.45 | 466.03 | 420.79 |
| Serpin D1 | 1.16 | 11.46 | 434.52 | 239.48 |
| Serpin I1 | 1.16 | 6.19 | 510.88 | 365.53 |
| SERTAD2 | 1.16 | 85.84 | 597.65 | 580.78 |
| SHBG | 1.16 | 128.57 | 859.98 | 562.89 |
| SMAC | 1.16 | 20.76 | 700.13 | 268.20 |
| SPINK1 | 1.16 | 60.57 | 476.02 | 478.31 |
| SSEA-1 | 1.16 | 125.19 | 629.99 | 603.41 |
| SSEA-4 | 97.34 | 347.22 | 543.72 | 617.26 |
| SSTR5 | 1.16 | 238.95 | 761.82 | 622.90 |
| Syndecan-3 | 39.98 | 90.24 | 807.73 | 969.06 |
| TAF4 | 1.16 | 32.94 | 647.13 | 518.04 |
| TFF3 | 1.16 | 117.74 | 548.67 | 788.19 |
| Thrombin | 1.16 | 131.45 | 560.74 | 492.24 |
| Thrombomodulin | 1.16 | 108.75 | 488.74 | 359.09 |
| Thymidine | 1.16 | 84.36 | 529.55 | 418.31 |
| Kinase-1 | | | | |
| Thyroglobulin | 1.16 | 629.61 | 570.80 | 376.26 |
| TIM-1 | 1.16 | 134.86 | 564.46 | 450.91 |
| TOPORS | 1.16 | 156.34 | 667.07 | 423.96 |
| TPA | 1.16 | 158.32 | 707.80 | 364.07 |
| TPM1 | 1.16 | 167.65 | 783.88 | 407.91 |
| Trappin-2 | 48.67 | 120.79 | 542.44 | 524.63 |
| TSLP | 127.46 | 294.62 | 1,419.29 | 678.46 |
| Tyk2 | 58.52 | 176.40 | 951.65 | 589.09 |
| TYRO10 | 45.19 | 226.37 | 849.45 | 756.23 |
| Vasopressin | 1.16 | 141.88 | 670.52 | 460.77 |
| VDUP-1 | 16.80 | 163.28 | 906.36 | 600.84 |
| VEGF R1 | 1.16 | 144.03 | 609.10 | 429.33 |
| VGF | 1.16 | 71.98 | 563.05 | 315.72 |
| VIP Receptor 2 | 1.16 | 127.08 | 609.75 | 265.00 |
| Visfatin | 1.16 | 175.82 | 825.67 | 463.03 |
| Vitamin D-BP | 88.07 | 201.02 | 629.39 | 212.06 |
| Vitamin | 1.16 | 188.38 | 488.99 | 279.33 |
| K-dependent | | | | |
| protein S | | | | |

Table 2 shows that a total of 231/616 (37.5%) of the proteins identified as Biologically Significant relative to Control were shared across all three (3) ethnic groups. The remaining 385/616 (62.5%) proteins demonstrated Biologically Significance in 2 or fewer of the sample groups.

Table 3 is a comparison of average production levels of the Asian (As) samples compared to African (Af) samples in the 231 shared, biologically significant proteins. Using the criteria of ≧25% increase/decrease in relative production levels as described in the protocol, Asian sample proteins were assessed as increased (one asterisk, “*”) or decreased production (three asterisks, “***”) than African samples.

TABLE 3

| | | AS | AF |
|---|---|---|---|
| | CONTROL | Average | Average |
| BCMA/TNFRSF17 | 1.08 | 67.49*** | 208.10*** |
| BAX | 90.68 | 552.02* | 357.12* |
| beta-NGF | 199.51 | 358.85*** | 478.62*** |
| CCL14/HCC-1/HCC-3 | 482.66 | 74.14*** | 187.07*** |
| CCR5 | 71.19 | 143.29*** | 192.98*** |
| CD30/TNFRSF8 | 111.26 | 272.19*** | 446.20*** |
| CV-2/ | 108.01 | 18.99*** | 450.71*** |
| Crossveinless-2 | | | |
| CXCL16 | 44.67 | 293.17 | 389.68 |
| D6 | 216.83 | 435.25*** | 652.24*** |
| DAN | 686.77 | 1,597.31*** | 2,464.56*** |
| Decorin | 461.00 | 1,900.72*** | 4,089.23*** |
| Dkk-1 | 609.89 | 2,836.46 | 3,118.77 |
| Dkk-3 | 863.26 | 6,466.27 | 5,277.86 |
| EDA-A2 | 813.45 | 45,352.87*** | 86,934.18*** |
| EDAR | 578.49 | 1,715.75*** | 2,752.31*** |
| Eotaxin/CCL11 | 1,498.32 | 559.22 | 563.75 |
| FGF R5 | 674.85 | 1,554.38*** | 2,334.15*** |
| IGFBP-2 | 1,230.33 | 11,164.80*** | 22,591.35*** |
| IGFBP-3 | 1,211.38 | 4,239.29*** | 5,899.29*** |
| IGFBP-rp1/ | 11,808.74 | 76,254.45 | 90,204.78 |
| IGFBP-7 | | | |
| IL-5 R alpha | 1,489.12 | 3,137.10 | 3,075.82 |
| IL-12 R beta 2 | 2,022.94 | 4,457.84*** | 6,617.74*** |
| Latent | 922.82 | 4,029.66*** | 7,681.24*** |
| TGF-beta bp1 | | | |
| LFA-1 alpha | 611.51 | 1,354.79 | 1,250.51 |
| MIP-3 beta | 1,536.22 | 3,864.34 | 4,525.98 |
| MMP-1 | 974.25 | 46,620.53 | 53,622.33 |
| MMP-2 | 890.87 | 2,317.71 | 2,954.82 |
| MMP-3 | 871.92 | 6,219.51 | 7,061.18 |
| Nidogen-1 | 1,167.53 | 3,590.00 | 4,048.72 |
| NrCam | 1,193.52 | 2,442.48*** | 3,271.70*** |
| Osteoprotegerin/ | 974.25 | 2,120.02 | 2,149.40 |
| TNFRSF11B | | | |
| Progranulin | 653.74 | 1,963.25 | 2,328.68 |
| Prdx | 533.01 | 1,162.32 | 1,306.85 |
| Soggy-1 | 966.13 | 2,179.15 | 1,993.02 |
| Sonic Hedgehog | 704.63 | 1,508.09 | 1,550.51 |
| (Shh N-terminal) | | | |
| SPARC | 956.92 | 8,863.21 | 8,852.43 |
| Spinesin | 743.61 | 1,736.29 | 2,210.92 |
| TIMP-1 | 1,295.84 | 14,201.02 | 16,620.57 |
| TIMP-2 | 820.49 | 28,405.89* | 22,696.06* |
| TIMP-3 | 559.00 | 1,753.62 | 2,267.47 |
| TLR2 | 932.56 | 1,872.01 | 2,033.23 |
| 2B4 | 1.16 | 36.20* | 9.28* |
| 4-1BB | 1.16 | 44.55* | 19.13* |
| A1BG | 1.16 | 114.05* | 42.87* |
| A2M | 211.47 | 1,600.89* | 1,136.95* |
| ADAMTS-10 | 1,469.89 | 3,168.98 | 3,839.53 |
| ADAMTS-19 | 1.16 | 50.06*** | 361.51*** |
| ADAMTS-4 | 1.16 | 58.51*** | 313.92*** |
| ADAMTS-5 | 1.16 | 11.18*** | 286.18*** |
| ADAMTS-L2 | 1.16 | 69.63*** | 559.99*** |
| Adipsin | 1.16 | 1,233.42*** | 5,756.54*** |
| Aldolase A | 1.16 | 110.96 | 150.78 |
| Aldolase B | 1.16 | 33.13*** | 50.87*** |
| Aldolase C | 1.16 | 141.93* | 81.12* |
| ALK | 1.16 | 105.42* | 66.77* |
| Alpha 1 AG | 1.16 | 162.29 | 170.43 |
| Alpha 1 | 1.16 | 306.58* | 234.13* |
| Microglobulin | | | |
| ALPP | 183.08 | 513.50 | 455.35 |
| ApoB | 1.16 | 22.98*** | 389.11*** |
| ApoD | 1.16 | 270.70*** | 422.17*** |
| ApoE | 1.16 | 98.24*** | 439.38*** |
| ApoE3 | 1.16 | 119.91*** | 195.12*** |
| ApoH | 1.16 | 185.87 | 170.33 |
| ApoM | 1.16 | 178.23 | 219.26 |
| APP | 1.16 | 2,545.55 | 2,551.79 |
| ASPH | 1.16 | 204.00 | 204.89 |
| Attractin | 1.16 | 127.47 | 150.90 |
| B3GNT1 | 1.16 | 173.58 | 221.68 |
| BAF57 | 1.16 | 198.59 | 218.13 |
| BAFF | 1.16 | 201.77 | 264.89 |
| Beta 2M | 412.52 | 2,635.25*** | 3,669.88*** |
| Beta IG-H3 | 643.69 | 7,717.55 | 7,511.18 |
| BNP | 108.34 | 229.07*** | 393.92*** |
| C5/C5a | 1.16 | 48.23*** | 291.43*** |
| C8B | 2,329.69 | 18.40*** | 329.57*** |
| C9 | 3,010.47 | 359.91*** | 768.15*** |
| CA15-3 | 1.16 | 255.70 | 223.13 |
| CA19-9 | 1.16 | 301.47 | 335.34 |
| CA125 | 1.16 | 313.04 | 298.27 |
| Cadherin-13 | 1.16 | 314.86*** | 458.31*** |
| Calreticulin | 39.98 | 175.45*** | 256.30*** |
| Calsyntenin-1 | 1.16 | 137.85*** | 305.11*** |
| CART | 17.96 | 199.66*** | 269.76*** |
| CD59 | 989.58 | 31.08*** | 291.57*** |
| CD74 | 1.16 | 478.12 | 577.33 |
| CD79 alpha | 85.75 | 386.11*** | 594.13*** |
| CD90 | 1.16 | 193.34*** | 326.88*** |
| CEA | 48.09 | 107.86*** | 271.54*** |
| CEACAM-1 | 1.16 | 118.58*** | 272.14*** |
| Ceruloplasmin | 1.16 | 62.42*** | 210.45*** |
| CFHR2 | 1.16 | 118.91*** | 248.80*** |
| Chemerin | 1.16 | 211.16*** | 499.05*** |
| CHI3L1 | 97.34 | 386.78*** | 668.44*** |
| COCO | 115.88 | 42.75*** | 434.58*** |
| Complement | 1.16 | 29.70*** | 358.92*** |
| factor H | | | |
| Contactin-2 | 124.57 | 389.77 | 323.82 |
| CRP | 1.16 | 274.28*** | 622.99*** |
| CRTAM | 56.78 | 126.63*** | 402.34*** |
| CutA | 89.22 | 13.30*** | 291.89*** |
| Cyclin D1 | 52.72 | 19.48*** | 338.28*** |
| Cystatin C | 13.33 | 156.76*** | 371.89*** |
| Cytochrome C | 1.16 | 147.29*** | 447.82*** |
| Cytokeratin 8 | 1.74 | 186.03*** | 461.25*** |
| EphA2 | 1.16 | 148.42*** | 465.87*** |
| EphB4 | 1.16 | 170.38*** | 488.75*** |
| Factor XIII A | 18,130.02 | 48,560.06 | 59,351.02 |
| Factor XIII B | 690.04 | 1,868.35 | 2,488.59 |
| FAP | 1,625.17 | 129.78*** | 490.19*** |
| Fen 1 | 45.19 | 136.69*** | 484.46*** |
| Fetuin B | 1.16 | 200.35*** | 768.38*** |
| Fibrinogen | 530.13 | 9,202.76*** | 13,333.01*** |
| Fibrinopeptide A | 276.94 | 692.98*** | 1,348.75*** |
| Fibronectin | 550.41 | 18,570.43*** | 28,116.82*** |
| Ficolin-3 | 59.10 | 626.64*** | 1,014.16*** |
| Galectin-7 | 513.91 | 1,083.84*** | 1,486.76*** |
| gamma-Thrombin | 47.51 | 233.70*** | 586.83*** |
| GATA-4 | 2.32 | 54.24*** | 469.00*** |
| Gelsolin | 224.22 | 89.75*** | 544.17*** |
| GMNN | 1.16 | 245.25*** | 695.85*** |
| GPBB | 1.16 | 147.93*** | 713.46*** |
| GPI | 1.16 | 111.65*** | 607.64*** |
| GPX1 | 281.58 | 592.73*** | 829.28*** |
| GRP | 298.38 | 938.00 | 1,120.75 |
| GRP75 | 1.16 | 346.33 | 406.21 |
| Hemopexin | 99.65 | 246.91*** | 522.14*** |
| HSP20 | 81.11 | 168.70*** | 524.19*** |
| HSP27 | 265.94 | 954.61*** | 1,434.63*** |
| HSP40 | 214.95 | 104.23*** | 557.77*** |
| HSP60 | 213.79 | 83.41*** | 553.85*** |
| HSP70 | 2.90 | 16.42*** | 567.39*** |
| HSP90 | 145.42 | 20.96*** | 615.34*** |
| Kallikrein 8 | 1.16 | 228.78*** | 520.51*** |
| Kallikrein 10 | 23.18 | 244.84*** | 551.24*** |
| Kallikrein 11 | 1.16 | 169.94*** | 448.90*** |
| Kallikrein 14 | 1.16 | 218.18*** | 482.29*** |
| KCC3 | 1.16 | 140.90*** | 534.97*** |
| KCTD10 | 1.16 | 156.82*** | 573.66*** |
| KIF3B | 1.16 | 196.27*** | 701.09*** |
| KLF4 | 67.21 | 184.46*** | 773.71*** |
| LYVE-1 | 1.16 | 227.30*** | 497.99*** |
| LZTS1 | 101.97 | 221.95*** | 548.54*** |
| Mammaglobin A | 1.16 | 217.39*** | 428.96*** |
| Marapsin | 64.31 | 234.40*** | 378.03*** |
| MATK | 39.40 | 323.89*** | 628.50*** |
| MBL | 45.77 | 332.55*** | 576.16*** |
| MBL-2 | 151.80 | 421.26*** | 753.06*** |
| Mesothelin | 42.87 | 202.92*** | 629.06*** |
| Neuropeptide Y | 85.75 | 220.99*** | 680.95*** |
| NF1 | 1.16 | 175.96*** | 445.36*** |
| Notch-1 | 1.16 | 205.31*** | 429.90*** |
| NPTX1 | 1.16 | 261.23*** | 430.64*** |
| NPTXR | 1.16 | 215.70*** | 355.49*** |
| NR3C3 | 1.16 | 757.02* | 402.10* |
| OCT3/4 | 61.99 | 297.65*** | 514.87*** |
| PAI-1 | 20.86 | 701.04*** | 1,662.47*** |
| PGRP-S | 23.75 | 286.05*** | 626.66*** |
| PI 16 | 1.16 | 154.88*** | 632.80*** |
| PI 3Kinase | 37.66 | 129.52*** | 536.89*** |
| p85 beta | | | |
| PIM2 | 1.16 | 50.27*** | 360.55*** |
| PKM2 | 1.16 | 146.93*** | 398.24*** |
| Plasminogen | 1.16 | 1,137.55*** | 3,677.66*** |
| Podocalyxin | 1.16 | 189.31*** | 509.40*** |
| PON1 | 1.16 | 304.75*** | 492.62*** |
| PON2 | 1.16 | 110.61*** | 499.41*** |
| PPARg2 | 53.30 | 214.39*** | 683.36*** |
| PPP2R5C | 133.26 | 295.86*** | 929.31*** |
| Pro-Cathepsin B | 68.95 | 299.96*** | 745.63*** |
| Progesterone | 1.16 | 68.74*** | 446.96*** |
| pro-Glucagon | 314.60 | 131.63*** | 689.57*** |
| Prohibitin | 1.16 | 84.54*** | 303.03*** |
| Pro-MMP-7 | 1.16 | 110.58*** | 328.87*** |
| ProSAAS | 121.09 | 306.45*** | 617.31*** |
| PSP | 1.16 | 125.16*** | 455.47*** |
| PTH | 1.16 | 533.71*** | 845.09*** |
| PTHLP | 7,014.00 | 30,003.94 | 25,623.63 |
| PTN | 1.16 | 815.10*** | 1,204.22*** |
| PYK2 | 1.16 | 122.90*** | 485.82*** |
| PYY | 165.12 | 386.93*** | 1,005.10*** |
| Ras | 66.63 | 3,697.36*** | 8,124.49*** |
| RBP4 | 1.16 | 195.55*** | 747.93*** |
| RECK | 1.16 | 240.27*** | 647.48*** |
| Resistin | 1.16 | 125.17*** | 600.75*** |
| RET | 1.16 | 232.98*** | 735.07*** |
| RIP1 | 1.16 | 115.00*** | 672.18*** |
| ROR2 | 51.56 | 250.44*** | 510.09*** |
| ROS | 34.18 | 272.58*** | 805.13*** |
| S100A4 | 25.49 | 106.27*** | 548.25*** |
| S100A6 | 1.16 | 167.09*** | 655.29*** |
| S100A8 | 1.16 | 63.15*** | 530.90*** |
| S-100b | 19.70 | 66.68*** | 358.95*** |
| SCG3 | 35.92 | 135.93*** | 542.17*** |
| Serpin A3 | 310.55 | 932.66 | 1,181.20 |
| Serpin A4 | 1.16 | 119.99*** | 622.57*** |
| Serpin A5 | 1.16 | 55.56*** | 559.85*** |
| Serpin A9 | 1.16 | 19.85*** | 496.21*** |
| Serpin A12 | 1.16 | 125.63*** | 589.49*** |
| Serpin B5 | 1.16 | 66.45*** | 466.03*** |
| Serpin D1 | 1.16 | 11.46*** | 434.52*** |
| Serpin I1 | 1.16 | 6.19*** | 510.88*** |
| SERTAD2 | 1.16 | 85.84*** | 597.65*** |
| SHBG | 1.16 | 128.57*** | 859.98*** |
| SMAC | 1.16 | 20.76*** | 700.13*** |
| SPINK1 | 1.16 | 60.57*** | 476.02*** |
| SSEA-1 | 1.16 | 125.19*** | 629.99*** |
| SSEA-4 | 97.34 | 347.22*** | 543.72*** |
| SSTR5 | 1.16 | 238.95*** | 761.82*** |
| Syndecan-3 | 39.98 | 90.24*** | 807.73*** |
| TAF4 | 1.16 | 32.94*** | 647.13*** |
| TFF3 | 1.16 | 117.74*** | 548.67*** |
| Thrombin | 1.16 | 131.45*** | 560.74*** |
| Thrombomodulin | 1.16 | 108.75*** | 488.74*** |
| Thymidine | 1.16 | 84.36*** | 529.55*** |
| Kinase-1 | | | |
| Thyroglobulin | 1.16 | 629.61 | 570.80 |
| TIM-1 | 1.16 | 134.86*** | 564.46*** |
| TOPORS | 1.16 | 156.34*** | 667.07*** |
| TPA | 1.16 | 158.32*** | 707.80*** |
| TPM1 | 1.16 | 167.65*** | 783.88*** |
| Trappin-2 | 48.67 | 120.79*** | 542.44*** |
| TSLP | 127.46 | 294.62*** | 1,419.29*** |
| Tyk2 | 58.52 | 176.40*** | 951.65*** |
| TYRO10 | 45.19 | 226.37*** | 849.45*** |
| Vasopressin | 1.16 | 141.88*** | 670.52*** |
| VDUP-1 | 16.80 | 163.28*** | 906.36*** |
| VEGF R1 | 1.16 | 144.03*** | 609.10*** |
| VGF | 1.16 | 71.98*** | 563.05*** |
| VIP Receptor 2 | 1.16 | 127.08*** | 609.75*** |
| Visfatin | 1.16 | 175.82*** | 825.67*** |
| Vitamin D-BP | 88.07 | 201.02*** | 629.39*** |
| Vitamin | 1.16 | 188.38*** | 488.99*** |
| K-dependent | | | |
| protein S | | | |

Table 3 shows that Asian samples relative to African samples demonstrated 25% difference in production levels in 183/231 (78%) of the compared proteins. Of those, only 10/182 (5%) demonstrated increase production levels relative to African samples. The remaining 172/182 (95%) demonstrated decreased production levels relative to African samples.

Table 4 is a comparison of average production levels of the Caucasian (Ca) samples compared to African (Af) samples in the 231 shared, biologically significant proteins. Using the criteria of ≧25% increase/decrease in relative production levels as described in the protocol, Caucasian (Ca) sample proteins were assessed as increased (one asterisk, “*”) or decreased production (three asterisks, “***”) than African samples.

TABLE 4

| | | AF | CA |
|---|---|---|---|
| | CONTROL | Average | Average |
| BCMA/TNFRSF17 | 1.08 | 208.10 | 194.35 |
| BAX | 90.68 | 357.12* | 759.17* |
| beta-NGF | 199.51 | 478.62* | 897.43* |
| CCL14/HCC-1/HCC-3 | 482.66 | 187.07* | 236.13* |
| CCR5 | 71.19 | 192.98* | 313.52* |
| CD30/TNFRSF8 | 111.26 | 446.20 | 489.25 |
| CV-2/ | 108.01 | 450.71*** | 45.32*** |
| Crossveinless-2 | | | |
| CXCL16 | 44.67 | 389.68 | 380.72 |
| D6 | 216.83 | 652.24*** | 453.43*** |
| DAN | 686.77 | 2,464.56 | 2,888.04 |
| Decorin | 461.00 | 4,089.23 | 3,161.92 |
| Dkk-1 | 609.89 | 3,118.77 | 3,029.36 |
| Dkk-3 | 863.26 | 5,277.86* | 6,745.95* |
| EDA-A2 | 813.45 | 86,934.18 | 102,337.42 |
| EDAR | 578.49 | 2,752.31* | 3,558.44* |
| Eotaxin/CCL11 | 1,498.32 | 563.75 | 583.23 |
| FGF R5 | 674.85 | 2,334.15 | 2,276.60 |
| IGFBP-2 | 1,230.33 | 22,591.35 | 23,988.15 |
| IGFBP-3 | 1,211.38 | 5,899.29* | 7,880.22* |
| IGFBP-rp1/ | 11,808.74 | 90,204.78 | 94,029.81 |
| IGFBP-7 | | | |
| IL-5 R alpha | 1,489.12 | 3,075.82* | 3,982.92* |
| IL-12 R beta 2 | 2,022.94 | 6,617.74 | 7,987.21 |
| Latent | 922.82 | 7,681.24 | 6,544.87 |
| TGF-beta bp1 | | | |
| LFA-1 alpha | 611.51 | 1,250.51 | 1,524.15 |
| MIP-3 beta | 1,536.22 | 4,525.98 | 5,350.64 |
| MMP-1 | 974.25 | 53,622.33*** | 38,420.38*** |
| MMP-2 | 890.87 | 2,954.82 | 2,737.83 |
| MMP-3 | 871.92 | 7,061.18 | 8,496.31 |
| Nidogen-1 | 1,167.53 | 4,048.72 | 4,497.40 |
| NrCam | 1,193.52 | 3,271.70 | 3,210.07 |
| Osteoprotegerin/ | 974.25 | 2,149.40* | 3,157.01* |
| TNFRSF11B | | | |
| Progranulin | 653.74 | 2,328.68*** | 1,521.58*** |
| Prdx | 533.01 | 1,306.85 | 1,280.77 |
| Soggy-1 | 966.13 | 1,993.02 | 2,347.68 |
| Sonic Hedgehog | 704.63 | 1,550.51 | 1,747.30 |
| (Shh N-terminal) | | | |
| SPARC | 956.92 | 8,852.43* | 14,857.38* |
| Spinesin | 743.61 | 2,210.92 | 2,700.87 |
| TIMP-1 | 1,295.84 | 16,620.57* | 25,661.55* |
| TIMP-2 | 820.49 | 22,696.06 | 26,642.47 |
| TIMP-3 | 559.00 | 2,267.47 | 2,468.84 |
| TLR2 | 932.56 | 2,033.23* | 2,700.80* |
| 2B4 | 1.16 | 9.28* | 73.02* |
| 4-1BB | 1.16 | 19.13* | 67.57* |
| A1BG | 1.16 | 42.87* | 115.12* |
| A2M | 211.47 | 1,136.95* | 2,328.91* |
| ADAMTS-10 | 1,469.89 | 3,839.53* | 5,165.18* |
| ADAMTS-19 | 1.16 | 361.51*** | 207.95*** |
| ADAMTS-4 | 1.16 | 313.92*** | 161.25*** |
| ADAMTS-5 | 1.16 | 286.18 | 218.20 |
| ADAMTS-L2 | 1.16 | 559.99* | 726.19* |
| Adipsin | 1.16 | 5,756.54*** | 1,943.35*** |
| Aldolase A | 1.16 | 150.78 | 104.91 |
| Aldolase B | 1.16 | 50.87*** | 26.21*** |
| Aldolase C | 1.16 | 81.12 | 79.57 |
| ALK | 1.16 | 66.77* | 96.93* |
| Alpha 1 AG | 1.16 | 170.43* | 284.78* |
| Alpha 1 | 1.16 | 234.13* | 591.28* |
| Microglobulin | | | |
| ALPP | 183.08 | 455.35* | 786.07* |
| ApoB | 1.16 | 389.11 | 401.33 |
| ApoD | 1.16 | 422.17 | 411.17 |
| ApoE | 1.16 | 439.38*** | 15.84*** |
| ApoE3 | 1.16 | 195.12*** | 22.22*** |
| ApoH | 1.16 | 170.33*** | 65.28*** |
| ApoM | 1.16 | 219.26 | 187.02 |
| APP | 1.16 | 2,551.79 | 2,560.93 |
| ASPH | 1.16 | 204.89 | 174.64 |
| Attractin | 1.16 | 150.90 | 118.11 |
| B3GNT1 | 1.16 | 221.68 | 198.86 |
| BAF57 | 1.16 | 218.13 | 216.41 |
| BAFF | 1.16 | 264.89 | 252.07 |
| Beta 2M | 412.52 | 3,669.88 | 3,451.24 |
| Beta IG-H3 | 643.69 | 7,511.18* | 18,306.78* |
| BNP | 108.34 | 393.92 | 406.62 |
| C5/C5a | 1.16 | 291.43 | 211.11 |
| C8B | 2,329.69 | 329.57* | 450.97* |
| C9 | 3,010.47 | 768.15* | 1,482.85* |
| CA15-3 | 1.16 | 223.13* | 369.19* |
| CA19-9 | 1.16 | 335.34*** | 153.30*** |
| CA125 | 1.16 | 298.27*** | 166.63*** |
| Cadherin-13 | 1.16 | 458.31 | 413.20 |
| Calreticulin | 39.98 | 256.30 | 252.87 |
| Calsyntenin-1 | 1.16 | 305.11*** | 182.14*** |
| CART | 17.96 | 269.76 | 316.66 |
| CD59 | 989.58 | 291.57* | 391.36* |
| CD74 | 1.16 | 577.33 | 721.31 |
| CD79 alpha | 85.75 | 594.13 | 603.35 |
| CD90 | 1.16 | 326.88 | 245.74 |
| CEA | 48.09 | 271.54 | 324.26 |
| CEACAM-1 | 1.16 | 272.14* | 350.90* |
| Ceruloplasmin | 1.16 | 210.45 | 231.17 |
| CFHR2 | 1.16 | 248.80 | 305.94 |
| Chemerin | 1.16 | 499.05 | 560.39 |
| CHI3L1 | 97.34 | 668.44* | 1,240.82* |
| COCO | 115.88 | 434.58 | 442.03 |
| Complement | 1.16 | 358.92 | 357.02 |
| factor H | | | |
| Contactin-2 | 124.57 | 323.82 | 292.41 |
| CRP | 1.16 | 622.99 | 499.57 |
| CRTAM | 56.78 | 402.34*** | 252.36*** |
| CutA | 89.22 | 291.89 | 237.14 |
| Cyclin D1 | 52.72 | 338.28 | 281.74 |
| Cystatin C | 13.33 | 371.89 | 435.83 |
| Cytochrome C | 1.16 | 447.82 | 533.46 |
| Cytokeratin 8 | 1.74 | 461.25 | 570.57 |
| EphA2 | 1.16 | 465.87*** | 299.77*** |
| EphB4 | 1.16 | 488.75 | 535.43 |
| Factor XIII A | 18,130.02 | 59,351.02* | 79,043.79* |
| Factor XIII B | 690.04 | 2,488.59* | 3,152.99* |
| FAP | 1,625.17 | 490.19 | 468.82 |
| Fen 1 | 45.19 | 484.46 | 581.16 |
| Fetuin B | 1.16 | 768.38*** | 569.76*** |
| Fibrinogen | 530.13 | 13,333.01* | 21,925.77* |
| Fibrinopeptide A | 276.94 | 1,348.75* | 2,031.59* |
| Fibronectin | 550.41 | 28,116.82* | 41,847.45* |
| Ficolin-3 | 59.10 | 1,014.16* | 1,362.72* |
| Galectin-7 | 513.91 | 1,486.76* | 2,699.46* |
| gamma-Thrombin | 47.51 | 586.83 | 667.71 |
| GATA-4 | 2.32 | 469.00 | 453.90 |
| Gelsolin | 224.22 | 544.17 | 607.43 |
| GMNN | 1.16 | 695.85 | 527.85 |
| GPBB | 1.16 | 713.46 | 545.19 |
| GPI | 1.16 | 607.64*** | 405.04*** |
| GPX1 | 281.58 | 829.28 | 926.51 |
| GRP | 298.38 | 1,120.75* | 1,747.49* |
| GRP75 | 1.16 | 406.21* | 766.37* |
| Hemopexin | 99.65 | 522.14 | 645.26 |
| HSP20 | 81.11 | 524.19 | 631.09 |
| HSP27 | 265.94 | 1,434.63 | 1,441.15 |
| HSP40 | 214.95 | 557.77 | 696.00 |
| HSP60 | 213.79 | 553.85* | 708.74* |
| HSP70 | 2.90 | 567.39* | 716.37* |
| HSP90 | 145.42 | 615.34 | 588.50 |
| Kallikrein 8 | 1.16 | 520.51 | 526.08 |
| Kallikrein 10 | 23.18 | 551.24 | 672.79 |
| Kallikrein 11 | 1.16 | 448.90 | 517.98 |
| Kallikrein 14 | 1.16 | 482.29 | 582.76 |
| KCC3 | 1.16 | 534.97 | 476.73 |
| KCTD10 | 1.16 | 573.66 | 604.07 |
| KIF3B | 1.16 | 701.09* | 894.29* |
| KLF4 | 67.21 | 773.71* | 1,038.51* |
| LYVE-1 | 1.16 | 497.99 | 532.83 |
| LZTS1 | 101.97 | 548.54* | 757.35* |
| Mammaglobin A | 1.16 | 428.96* | 742.71* |
| Marapsin | 64.31 | 378.03* | 602.72* |
| MATK | 39.40 | 628.50 | 759.82 |
| MBL | 45.77 | 576.16* | 784.43* |
| MBL-2 | 151.80 | 753.06* | 1,394.77* |
| Mesothelin | 42.87 | 629.06 | 619.08 |
| Neuropeptide Y | 85.75 | 680.95 | 540.43 |
| NF1 | 1.16 | 445.36 | 457.17 |
| Notch-1 | 1.16 | 429.90 | 455.91 |
| NPTX1 | 1.16 | 430.64 | 465.00 |
| NPTXR | 1.16 | 355.49* | 514.51* |
| NR3C3 | 1.16 | 402.10 | 431.06 |
| OCT3/4 | 61.99 | 514.87 | 566.50 |
| PAI-1 | 20.86 | 1,662.47* | 2,454.59* |
| PGRP-S | 23.75 | 626.66* | 877.90* |
| PI 16 | 1.16 | 632.80* | 830.47* |
| PI 3Kinase | 37.66 | 536.89 | 590.47 |
| p85 beta | | | |
| PIM2 | 1.16 | 360.55 | 363.27 |
| PKM2 | 1.16 | 398.24* | 551.75* |
| Plasminogen | 1.16 | 3,677.66 | 3,358.75 |
| Podocalyxin | 1.16 | 509.40 | 505.68 |
| PON1 | 1.16 | 492.62 | 452.95 |
| PON2 | 1.16 | 499.41* | 690.37* |
| PPARg2 | 53.30 | 683.36 | 575.00 |
| PPP2R5C | 133.26 | 929.31 | 915.39 |
| Pro-Cathepsin B | 68.95 | 745.63*** | 529.31*** |
| Progesterone | 1.16 | 446.96 | 353.70 |
| pro-Glucagon | 314.60 | 689.57* | 1,104.88* |
| Prohibitin | 1.16 | 303.03 | 379.16 |
| Pro-MMP-7 | 1.16 | 328.87 | 359.80 |
| ProSAAS | 121.09 | 617.31 | 523.80 |
| PSP | 1.16 | 455.47* | 589.44* |
| PTH | 1.16 | 845.09* | 2,143.04* |
| PTHLP | 7,014.00 | 25,623.63* | 43,869.68* |
| PTN | 1.16 | 1,204.22* | 1,688.76* |
| PYK2 | 1.16 | 485.82* | 801.01* |
| PYY | 165.12 | 1,005.10* | 1,993.06* |
| Ras | 66.63 | 8,124.49* | 14,353.17* |
| RBP4 | 1.16 | 747.93 | 748.67 |
| RECK | 1.16 | 647.48 | 692.97 |
| Resistin | 1.16 | 600.75 | 581.38 |
| RET | 1.16 | 735.07 | 651.36 |
| RIP1 | 1.16 | 672.18*** | 355.42*** |
| ROR2 | 51.56 | 510.09*** | 265.49*** |
| ROS | 34.18 | 805.13 | 692.06 |
| S100A4 | 25.49 | 548.25 | 557.05 |
| S100A6 | 1.16 | 655.29 | 755.59 |
| S100A8 | 1.16 | 530.90 | 625.94 |
| S-100b | 19.70 | 358.95 | 481.14 |
| SCG3 | 35.92 | 542.17 | 474.98 |
| Serpin A3 | 310.55 | 1,181.20* | 1,906.23* |
| Serpin A4 | 1.16 | 622.57 | 642.78 |
| Serpin A5 | 1.16 | 559.85 | 669.43 |
| Serpin A9 | 1.16 | 496.21 | 526.73 |
| Serpin A12 | 1.16 | 589.49* | 761.49* |
| Serpin B5 | 1.16 | 466.03 | 420.79 |
| Serpin D1 | 1.16 | 434.52*** | 239.48*** |
| Serpin I1 | 1.16 | 510.88*** | 365.53*** |
| SERTAD2 | 1.16 | 597.65 | 580.78 |
| SHBG | 1.16 | 859.98*** | 562.89*** |
| SMAC | 1.16 | 700.13*** | 268.20*** |
| SPINK1 | 1.16 | 476.02 | 478.31 |
| SSEA-1 | 1.16 | 629.99 | 603.41 |
| SSEA-4 | 97.34 | 543.72 | 617.26 |
| SSTR5 | 1.16 | 761.82 | 622.90 |
| Syndecan-3 | 39.98 | 807.73 | 969.06 |
| TAF4 | 1.16 | 647.13 | 518.04 |
| TFF3 | 1.16 | 548.67* | 788.19* |
| Thrombin | 1.16 | 560.74 | 492.24 |
| Thrombomodulin | 1.16 | 488.74*** | 359.09*** |
| Thymidine | 1.16 | 529.55 | 418.31 |
| Kinase-1 | | | |
| Thyroglobulin | 1.16 | 570.80*** | 376.26*** |
| TIM-1 | 1.16 | 564.46 | 450.91 |
| TOPORS | 1.16 | 667.07*** | 423.96*** |
| TPA | 1.16 | 707.80*** | 364.07*** |
| TPM1 | 1.16 | 783.88*** | 407.91*** |
| Trappin-2 | 48.67 | 542.44 | 524.63 |
| TSLP | 127.46 | 1,419.29*** | 678.46*** |
| Tyk2 | 58.52 | 951.65*** | 589.09*** |
| TYRO10 | 45.19 | 849.45 | 756.23 |
| Vasopressin | 1.16 | 670.52*** | 460.77*** |
| VDUP-1 | 16.80 | 906.36*** | 600.84*** |
| VEGF R1 | 1.16 | 609.10*** | 429.33*** |
| VGF | 1.16 | 563.05*** | 315.72*** |
| VIP Receptor 2 | 1.16 | 609.75*** | 265.00*** |
| Visfatin | 1.16 | 825.67*** | 463.03*** |
| Vitamin D-BP | 88.07 | 629.39*** | 212.06*** |
| Vitamin | 1.16 | 488.99*** | 279.33*** |
| K-dependent | | | |
| protein S | | | |

Table 4 shows that Caucasian samples relative to African samples demonstrated 25% difference in production levels in 104/231 (45%) of the compared proteins. Of those, only 64/104 (62%) demonstrated increase production levels relative to African samples. The remaining 40/104 (38%) demonstrated decreased production levels relative to African samples.

Table 5 is a comparison of average production levels of the Caucasian (Ca) samples compared to Asian (As) samples in the 231 shared, biologically significant proteins. Using the criteria of ≧25% increase/decrease in relative production levels as described in the protocol, Caucasian (Ca) sample proteins were assessed as increased (one asterisk, “*”) or decreased production (three asterisks, “***”) than African samples.

TABLE 5

| | | AS | CA |
|---|---|---|---|
| | CONTROL | Average | Average |
| BCMA/TNFRSF17 | 1.08 | 67.49* | 194.35* |
| BAX | 90.68 | 552.02* | 759.17* |
| beta-NGF | 199.51 | 358.85* | 897.43* |
| CCL14/HCC-1/HCC-3 | 482.66 | 74.14* | 236.13* |
| CCR5 | 71.19 | 143.29* | 313.52* |
| CD30/TNFRSF8 | 111.26 | 272.19* | 489.25* |
| CV-2/ | 108.01 | 18.99* | 45.32* |
| Crossveinless-2 | | | |
| CXCL16 | 44.67 | 293.17* | 380.72* |
| D6 | 216.83 | 435.25 | 453.43 |
| DAN | 686.77 | 1,597.31* | 2,888.04* |
| Decorin | 461.00 | 1,900.72* | 3,161.92* |
| Dkk-1 | 609.89 | 2,836.46 | 3,029.36 |
| Dkk-3 | 863.26 | 6,466.27 | 6,745.95 |
| EDA-A2 | 813.45 | 45,352.87* | 102,337.42* |
| EDAR | 578.49 | 1,715.75* | 3,558.44* |
| Eotaxin/CCL11 | 1,498.32 | 559.22 | 583.23 |
| FGF R5 | 674.85 | 1,554.38* | 2,276.60* |
| IGFBP-2 | 1,230.33 | 11,164.80* | 23,988.15* |
| IGFBP-3 | 1,211.38 | 4,239.29* | 7,880.22* |
| IGFBP-rp1/ | 11,808.74 | 76,254.45 | 94,029.81 |
| IGFBP-7 | | | |
| IL-5 R alpha | 1,489.12 | 3,137.10* | 3,982.92* |
| IL-12 R beta 2 | 2,022.94 | 4,457.84* | 7,987.21* |
| Latent | 922.82 | 4,029.66* | 6,544.87* |
| TGF-beta bp1 | | | |
| LFA-1 alpha | 611.51 | 1,354.79 | 1,524.15 |
| MIP-3 beta | 1,536.22 | 3,864.34* | 5,350.64* |
| MMP-1 | 974.25 | 46,620.53 | 38,420.38 |
| MMP-2 | 890.87 | 2,317.71 | 2,737.83 |
| MMP-3 | 871.92 | 6,219.51* | 8,496.31* |
| Nidogen-1 | 1,167.53 | 3,590.00* | 4,497.40* |
| NrCam | 1,193.52 | 2,442.48* | 3,210.07* |
| Osteoprotegerin/ | 974.25 | 2,120.02* | 3,157.01* |
| TNFRSF11B | | | |
| Progranulin | 653.74 | 1,963.25 | 1,521.58 |
| Prdx | 533.01 | 1,162.32 | 1,280.77 |
| Soggy-1 | 966.13 | 2,179.15 | 2,347.68 |
| Sonic Hedgehog | 704.63 | 1,508.09 | 1,747.30 |
| (Shh N-terminal) | | | |
| SPARC | 956.92 | 8,863.21* | 14,857.38* |
| Spinesin | 743.61 | 1,736.29* | 2,700.87* |
| TIMP-1 | 1,295.84 | 14,201.02* | 25,661.55* |
| TIMP-2 | 820.49 | 28,405.89 | 26,642.47 |
| TIMP-3 | 559.00 | 1,753.62* | 2,468.84* |
| TLR2 | 932.56 | 1,872.01* | 2,700.80* |
| 2B4 | 1.16 | 36.20* | 73.02* |
| 4-1BB | 1.16 | 44.55* | 67.57* |
| A1BG | 1.16 | 114.05 | 115.12 |
| A2M | 211.47 | 1,600.89* | 2,328.91* |
| ADAMTS-10 | 1,469.89 | 3,168.98* | 5,165.18* |
| ADAMTS-19 | 1.16 | 50.06* | 207.95* |
| ADAMTS-4 | 1.16 | 58.51* | 161.25* |
| ADAMTS-5 | 1.16 | 11.18* | 218.20* |
| ADAMTS-L2 | 1.16 | 69.63* | 726.19* |
| Adipsin | 1.16 | 1,233.42* | 1,943.35* |
| Aldolase A | 1.16 | 110.96 | 104.91 |
| Aldolase B | 1.16 | 33.13 | 26.21 |
| Aldolase C | 1.16 | 141.93*** | 79.57*** |
| ALK | 1.16 | 105.42 | 96.93 |
| Alpha 1 AG | 1.16 | 162.29* | 284.78* |
| Alpha 1 | 1.16 | 306.58* | 591.28* |
| Microglobulin | | | |
| ALPP | 183.08 | 513.50* | 786.07* |
| ApoB | 1.16 | 22.98* | 401.33* |
| ApoD | 1.16 | 270.70* | 411.17* |
| ApoE | 1.16 | 98.24*** | 15.84*** |
| ApoE3 | 1.16 | 119.91*** | 22.22*** |
| ApoH | 1.16 | 185.87*** | 65.28*** |
| ApoM | 1.16 | 178.23 | 187.02 |
| APP | 1.16 | 2,545.55 | 2,560.93 |
| ASPH | 1.16 | 204.00 | 174.64 |
| Attractin | 1.16 | 127.47 | 118.11 |
| B3GNT1 | 1.16 | 173.58 | 198.86 |
| BAF57 | 1.16 | 198.59 | 216.41 |
| BAFF | 1.16 | 201.77 | 252.07 |
| Beta 2M | 412.52 | 2,635.25* | 3,451.24* |
| Beta IG-H3 | 643.69 | 7,717.55* | 18,306.78* |
| BNP | 108.34 | 229.07* | 406.62* |
| C5/C5a | 1.16 | 48.23* | 211.11* |
| C8B | 2,329.69 | 18.40* | 450.97* |
| C9 | 3,010.47 | 359.91* | 1,482.85* |
| CA15-3 | 1.16 | 255.70* | 369.19* |
| CA19-9 | 1.16 | 301.47*** | 153.30*** |
| CA125 | 1.16 | 313.04*** | 166.63*** |
| Cadherin-13 | 1.16 | 314.86* | 413.20* |
| Calreticulin | 39.98 | 175.45* | 252.87* |
| Calsyntenin-1 | 1.16 | 137.85* | 182.14* |
| CART | 17.96 | 199.66* | 316.66* |
| CD59 | 989.58 | 31.08* | 391.36* |
| CD74 | 1.16 | 478.12* | 721.31* |
| CD79 alpha | 85.75 | 386.11* | 603.35* |
| CD90 | 1.16 | 193.34* | 245.74* |
| CEA | 48.09 | 107.86* | 324.26* |
| CEACAM-1 | 1.16 | 118.58* | 350.90* |
| Ceruloplasmin | 1.16 | 62.42* | 231.17* |
| CFHR2 | 1.16 | 118.91* | 305.94* |
| Chemerin | 1.16 | 211.16* | 560.39* |
| CHI3L1 | 97.34 | 386.78* | 1,240.82* |
| COCO | 115.88 | 42.75* | 442.03* |
| Complement | 1.16 | 29.70* | 357.02* |
| factor H | | | |
| Contactin-2 | 124.57 | 389.77 | 292.41 |
| CRP | 1.16 | 274.28* | 499.57* |
| CRTAM | 56.78 | 126.63* | 252.36* |
| CutA | 89.22 | 13.30* | 237.14* |
| Cyclin D1 | 52.72 | 19.48* | 281.74* |
| Cystatin C | 13.33 | 156.76* | 435.83* |
| Cytochrome C | 1.16 | 147.29* | 533.46* |
| Cytokeratin 8 | 1.74 | 186.03* | 570.57* |
| EphA2 | 1.16 | 148.42* | 299.77* |
| EphB4 | 1.16 | 170.38* | 535.43* |
| Factor XIII A | 18,130.02 | 48,560.06* | 79,043.79* |
| Factor XIII B | 690.04 | 1,868.35* | 3,152.99* |
| FAP | 1,625.17 | 129.78* | 468.82* |
| Fen 1 | 45.19 | 136.69* | 581.16* |
| Fetuin B | 1.16 | 200.35* | 569.76* |
| Fibrinogen | 530.13 | 9,202.76* | 21,925.77* |
| Fibrinopeptide A | 276.94 | 692.98* | 2,031.59* |
| Fibronectin | 550.41 | 18,570.43* | 41,847.45* |
| Ficolin-3 | 59.10 | 626.64* | 1,362.72* |
| Galectin-7 | 513.91 | 1,083.84* | 2,699.46* |
| gamma-Thrombin | 47.51 | 233.70* | 667.71* |
| GATA-4 | 2.32 | 54.24* | 453.90* |
| Gelsolin | 224.22 | 89.75* | 607.43* |
| GMNN | 1.16 | 245.25* | 527.85* |
| GPBB | 1.16 | 147.93* | 545.19* |
| GPI | 1.16 | 111.65* | 405.04* |
| GPX1 | 281.58 | 592.73* | 926.51* |
| GRP | 298.38 | 938.00* | 1,747.49* |
| GRP75 | 1.16 | 346.33* | 766.37* |
| Hemopexin | 99.65 | 246.91* | 645.26* |
| HSP20 | 81.11 | 168.70* | 631.09* |
| HSP27 | 265.94 | 954.61* | 1,441.15* |
| HSP40 | 214.95 | 104.23* | 696.00* |
| HSP60 | 213.79 | 83.41* | 708.74* |
| HSP70 | 2.90 | 16.42* | 716.37* |
| HSP90 | 145.42 | 20.96* | 588.50* |
| Kallikrein 8 | 1.16 | 228.78* | 526.08* |
| Kallikrein 10 | 23.18 | 244.84* | 672.79* |
| Kallikrein 11 | 1.16 | 169.94* | 517.98* |
| Kallikrein 14 | 1.16 | 218.18* | 582.76* |
| KCC3 | 1.16 | 140.90* | 476.73* |
| KCTD10 | 1.16 | 156.82* | 604.07* |
| KIF3B | 1.16 | 196.27* | 894.29* |
| KLF4 | 67.21 | 184.46* | 1,038.51* |
| LYVE-1 | 1.16 | 227.30* | 532.83* |
| LZTS1 | 101.97 | 221.95* | 757.35* |
| Mammaglobin A | 1.16 | 217.39* | 742.71* |
| Marapsin | 64.31 | 234.40* | 602.72* |
| MATK | 39.40 | 323.89* | 759.82* |
| MBL | 45.77 | 332.55* | 784.43* |
| MBL-2 | 151.80 | 421.26* | 1,394.77* |
| Mesothelin | 42.87 | 202.92* | 619.08* |
| Neuropeptide Y | 85.75 | 220.99* | 540.43* |
| NF1 | 1.16 | 175.96* | 457.17* |
| Notch-1 | 1.16 | 205.31* | 455.91* |
| NPTX1 | 1.16 | 261.23* | 465.00* |
| NPTXR | 1.16 | 215.70* | 514.51* |
| NR3C3 | 1.16 | 757.02*** | 431.06*** |
| OCT3/4 | 61.99 | 297.65* | 566.50* |
| PAI-1 | 20.86 | 701.04* | 2,454.59* |
| PGRP-S | 23.75 | 286.05* | 877.90* |
| PI 16 | 1.16 | 154.88* | 830.47* |
| PI 3Kinase | 37.66 | 129.52* | 590.47* |
| p85 beta | | | |
| PIM2 | 1.16 | 50.27* | 363.27* |
| PKM2 | 1.16 | 146.93* | 551.75* |
| Plasminogen | 1.16 | 1,137.55* | 3,358.75* |
| Podocalyxin | 1.16 | 189.31* | 505.68* |
| PON1 | 1.16 | 304.75* | 452.95* |
| PON2 | 1.16 | 110.61* | 690.37* |
| PPARg2 | 53.30 | 214.39* | 575.00* |
| PPP2R5C | 133.26 | 295.86* | 915.39* |
| Pro-Cathepsin B | 68.95 | 299.96* | 529.31* |
| Progesterone | 1.16 | 68.74* | 353.70* |
| pro-Glucagon | 314.60 | 131.63* | 1,104.88* |
| Prohibitin | 1.16 | 84.54* | 379.16* |
| Pro-MMP-7 | 1.16 | 110.58* | 359.80* |
| ProSAAS | 121.09 | 306.45* | 523.80* |
| PSP | 1.16 | 125.16* | 589.44* |
| PTH | 1.16 | 533.71* | 2,143.04* |
| PTHLP | 7,014.00 | 30,003.94* | 43,869.68* |
| PTN | 1.16 | 815.10* | 1,688.76* |
| PYK2 | 1.16 | 122.90* | 801.01* |
| PYY | 165.12 | 386.93* | 1,993.06* |
| Ras | 66.63 | 3,697.36* | 14,353.17* |
| RBP4 | 1.16 | 195.55* | 748.67* |
| RECK | 1.16 | 240.27* | 692.97* |
| Resistin | 1.16 | 125.17* | 581.38* |
| RET | 1.16 | 232.98* | 651.36* |
| RIP1 | 1.16 | 115.00* | 355.42* |
| ROR2 | 51.56 | 250.44 | 265.49 |
| ROS | 34.18 | 272.58* | 692.06* |
| S100A4 | 25.49 | 106.27* | 557.05* |
| S100A6 | 1.16 | 167.09* | 755.59* |
| S100A8 | 1.16 | 63.15* | 625.94* |
| S-100b | 19.70 | 66.68* | 481.14* |
| SCG3 | 35.92 | 135.93* | 474.98* |
| Serpin A3 | 310.55 | 932.66* | 1,906.23* |
| Serpin A4 | 1.16 | 119.99* | 642.78* |
| Serpin A5 | 1.16 | 55.56* | 669.43* |
| Serpin A9 | 1.16 | 19.85* | 526.73* |
| Serpin A12 | 1.16 | 125.63* | 761.49* |
| Serpin B5 | 1.16 | 66.45* | 420.79* |
| Serpin D1 | 1.16 | 11.46* | 239.48* |
| Serpin I1 | 1.16 | 6.19* | 365.53* |
| SERTAD2 | 1.16 | 85.84* | 580.78* |
| SHBG | 1.16 | 128.57* | 562.89* |
| SMAC | 1.16 | 20.76* | 268.20* |
| SPINK1 | 1.16 | 60.57* | 478.31* |
| SSEA-1 | 1.16 | 125.19* | 603.41* |
| SSEA-4 | 97.34 | 347.22* | 617.26* |
| SSTR5 | 1.16 | 238.95* | 622.90* |
| Syndecan-3 | 39.98 | 90.24* | 969.06* |
| TAF4 | 1.16 | 32.94* | 518.04* |
| TFF3 | 1.16 | 117.74* | 788.19* |
| Thrombin | 1.16 | 131.45* | 492.24* |
| Thrombomodulin | 1.16 | 108.75* | 359.09* |
| Thymidine | 1.16 | 84.36* | 418.31* |
| Kinase-1 | | | |
| Thyroglobulin | 1.16 | 629.61*** | 376.26*** |
| TIM-1 | 1.16 | 134.86* | 450.91* |
| TOPORS | 1.16 | 156.34* | 423.96* |
| TPA | 1.16 | 158.32* | 364.07* |
| TPM1 | 1.16 | 167.65* | 407.91* |
| Trappin-2 | 48.67 | 120.79* | 524.63* |
| TSLP | 127.46 | 294.62* | 678.46* |
| Tyk2 | 58.52 | 176.40* | 589.09* |
| TYRO10 | 45.19 | 226.37* | 756.23* |
| Vasopressin | 1.16 | 141.88* | 460.77* |
| VDUP-1 | 16.80 | 163.28* | 600.84* |
| VEGF R1 | 1.16 | 144.03* | 429.33* |
| VGF | 1.16 | 71.98* | 315.72* |
| VIP Receptor 2 | 1.16 | 127.08* | 265.00* |
| Visfatin | 1.16 | 175.82* | 463.03* |
| Vitamin D-BP | 88.07 | 201.02 | 212.06 |
| Vitamin | 1.16 | 188.38* | 279.33* |
| K-dependent | | | |
| protein S | | | |

Table 5 shows that Caucasian samples relative to Asian samples demonstrated 25% difference in production levels in 204/231 (88%) of the compared proteins. Of those, 196/204 (96%) demonstrated increase production levels relative to Asian samples. The remaining 8/204 (4%) demonstrated decreased production levels relative to Asian samples.

Table 6 is a list (and directional indication relative to Control) of Biologically Significant (≧2-fold dysregulation) proteins unique to each sample group.

TABLE 6

| Biologically | Biologically | Biologically |
|---|---|---|
| Significant Proteins | Significant Proteins | Significant Proteins |
| With Values Uniquely | With Values Uniquely | With Values Uniquely |
| Dysregulated Rela- | Dysregulated Rela- | Dysregulated Rela- |
| tive to Control In | tive to Control In | tive to Control In |
| Asian Group | African Group | Caucasian Group |
| 6Ckine ↓ | CXCR1 ↑ | Angiogenin ↑ |
| Angiopoietin 4 ↓ | CXCR4 ↑ | BIK ↑ |
| Angiopoietin Like 1 ↓ | Endostatin ↑ | BMP2 ↑ |
| Angiopoietin like 2 ↓ | GDF5 ↑ | BMP3 ↑ |
| Angiopoietin like | GFR Alpha3 ↑ | BMP3b ↑ |
| Factor↓ | GLUT3 ↑ | BMP4 ↑ |
| Angiostatin ↓ | Glypican 5 ↑ | BMP5 ↑ |
| BMP 15 ↓ | Musk ↑ | BMP6 ↑ |
| BMPR-1A/ALK3 ↓ | SMAD 4 ↑ | CCR7 ↑ |
| BMPR-1B/ALK 6 ↓ | SMAD 5 ↑ | CD40 Ligand ↑ |
| CCR1 ↓ | SMAD 7 ↑ | Cerberus 1 ↑ |
| CCR2 ↓ | BCAM ↑ | Chem R23 ↑ |
| CCR3 ↓ | Contactin ↑ | Chordin Like 2 ↑ |
| CRTH-2 ↓ | Cytokeratin 19 ↑ | CXCR6 ↑ |
| Cryptic ↓ | Endothelin RA ↑ | Follistatin ↑ |
| CTGF ↓ | GPR 39 ↑ | GFR-Alpha 2 ↑ |
| CTLA-4 ↓ | Osteocalcin ↑ | GITR ↑ |
| EG-VEGF ↓ | P21 ↑ | GITR Ligand ↑ |
| FAM 3B ↑ | Presenilin 1↑ | ICAM 3 ↑ |
| FGF8 ↓ | Presenilin 2 ↑ | ICAM 5 ↑ |
| GCSF ↓ | ROR1 ↑ | IL1 R9 ↑ |
| CD114 ↓ | SOX17 ↑ | IL1 RA ↑ |
| Hepassocin ↓ | SOX2 ↑ | IL1 sRI ↑ |
| GLO-1 ↓ | SYK ↑ | IL3 ↑ |
| HGF ↓ | TRA-1-60 ↑ | IL6 ↑ |
| HGF-R ↓ | TRA-1-81 ↑ | IL13 Alpha 2 ↑ |
| IL1-Beta ↓ | Transferrin ↑ | IL15R Alpha ↑ |
| IL1-F7 ↓ | Trypsin 1 ↑ | IL17 RD ↑ |
| IL1-F9 ↓ | | Leptin ↑ |
| MCP3 ↓ | | LIF ↑ |
| MCP4 ↓ | | LIF R Alpha ↑ |
| ROBO4 ↑ | | LRP1 ↑ |
| ADAMTS-13 ↓ | | MIP 1A ↑ |
| ADAMTS-15 ↓ | | MIP 3 Alpha ↑ |
| ADAMTS-17 ↓ | | NOV ↑ |
| Annexin A7 ↓ | | S100A8 ↑ |
| APC ↓ | | sFRP 4 ↑ |
| Apex1 ↓ | | TACI ↑ |
| ApoA2 ↓ | | Tarc ↑ |
| APoB100 ↓ | | TGF beta 3↑ |
| Apo C2 ↓ | | Thymopoeitin ↑ |
| BMP 9 ↓ | | TIE 2 ↑ |
| C3A ↓ | | TIMP 4 ↑ |
| C7 ↓ | | TL1A ↑ |
| Cathepsin L ↓ | | TLR1 ↑ |
| Cathepsin S ↓ | | TLR3 ↑ |
| CCK ↓ | | TLR4 ↑ |
| CD23 ↓ | | TMEFF1 ↑ |
| CD24 ↓ | | TNF Alpha ↑ |
| CD36 ↓ | | TNF Beta ↑ |
| CD38 ↓ | | TNF RI ↑ |
| CD44 ↓ | | TROY ↑ |
| CD45 ↓ | | uPA ↑ |
| CD46 ↓ | | uPAR ↑ |
| CD55 ↓ | | Vasorin ↑ |
| CD61 ↓ | | VCAM-1 ↑ |
| CK Beta 8-1 ↓ | | VE-Cadherin ↑ |
| CK-MB ↓ | | VEGF ↑ |
| Claudin 3 ↓ | | VEGF R2 ↑ |
| Claudin 4 ↓ | | VEGF R3 ↑ |
| CLEC3B ↓ | | VEGF B↑ |
| Clusterin ↓ | | XEDAR ↑ |
| CNDP1 ↓ | | ACE-2 ↑ |
| CPN2 ↓ | | ACPP ↑ |
| cTnT ↓ | | ACTH ↑ |
| DBI ↓ | | ADAM 9 ↑ |
| DCBLD2 ↓ | | AMICA ↑ |
| DEFA 1/3 ↓ | | AMPKa1 ↑ |
| Defensin ↓ | | CA9 ↑ |
| Desmin ↓ | | Calbindin D ↑ |
| DLL1 ↓ | | Caspase 8 ↑ |
| DLL4 ↓ | | Cathepsin B ↑ |
| E-Cadherin ↓ | | Cathepsin D ↑ |
| Endorphin Beta ↓ | | Chymase ↑ |
| EphA4 ↓ | | FGFR ↑ |
| EphA5 ↓ | | Galanin ↑ |
| AphA7 ↓ | | GRP78 ↑ |
| EphA8 ↓ | | GSR ↑ |
| EphB1 ↓ | | HADHA ↑ |
| EphB2 ↓ | | Hck ↑ |
| ESAM ↓ | | HOXA10 ↑ |
| EV15L ↓ | | HTRA2 ↑ |
| FABP1 ↓ | | IL-23p19 ↑ |
| FABP2 ↓ | | IL-33 ↑ |
| FAK ↓ | | LH ↑ |
| FcRIIB/C ↓ | | LPS ↑ |
| Ferritin ↓ | | LYRIC ↑ |
| Fetuin A ↓ | | PAK7 ↑ |
| FoxP3 ↓ | | Serpin A8 ↑ |
| Fyn ↓ | | |
| Gastrin ↓ | | |
| GATA3 ↓ | | |
| Ghrelin ↓ | | |
| GLP-1 ↓ | | |
| LAG-3 ↓ | | |
| Layilin ↓ | | |
| LDL R ↓ | | |
| Legumain ↓ | | |
| LRG ↓ | | |
| Midkine ↓ | | |
| MINA↓ | | |
| MSHa ↓ | | |
| MTUS1 ↓ | | |
| Myoglobin ↓ | | |
| NAIP ↓ | | |
| Nanog ↓ | | |
| Nestin ↓ | | |
| NET1 ↓ | | |
| Netrin-4 ↓ | | |
| P27 ↓ | | |
| P53 ↓ | | |
| Pancreastatin ↓ | | |
| Pancreatic Polypeptide ↓ | | |
| Pappalysin-1 ↓ | | |
| ROCK1 ↓ | | |
| SOST ↓ | | |

Table 6 shows that Asian samples demonstrated unique dysregulation (primarily less production than Control in all but two (2) instances) in 114/1000 {11.4%} proteins. African samples demonstrated unique dysregulation (all demonstrated greater production than Control) in 30/1000 {3%} proteins. Caucasian samples demonstrated unique dysregulation (all demonstrated greater production than Control) in 88/1000 {8.8%} proteins.

Table 7 is a list (with directional indication relative to comparator) of Statistically Significant (≧0.05) proteins for African relative to Asian.

TABLE 7

| | | Mann-Whitney U test |
|---|---|---|
| African Relative | Fold | p < 0.05, significant |
| to Asian | Change | difference |
| Contactin-1 | 4.785894 | 0.002 |
| RBP4 | 3.833005 | 0.002 |
| S100A8 | 8.433062 | 0.002 |
| Selenoprotein P | 464.1391 | 0.002 |
| SOX2 | 19.86845 | 0.002 |
| SRMS | 4.999207 | 0.002 |
| SSEA-1 | 5.044852 | 0.002 |
| TRA-1-60 | 14.72112 | 0.002 |
| Trappin-2 | 5.165469 | 0.002 |
| TSLP | 6.735374 | 0.002 |
| VDUP-1 | 5.84716 | 0.002 |
| GDF3 | 6.088395 | 0.004 |
| FGFR1 alpha | 8.376882 | 0.004 |
| PAI-1 | 2.413475 | 0.004 |
| pro-Glucagon | 20.06152 | 0.004 |
| RELM alpha | 4.326569 | 0.004 |
| Serpin A12 | 4.718214 | 0.004 |
| SPINK1 | 7.882159 | 0.004 |
| Tec | 27.83664 | 0.004 |
| Transferrin | 8.263479 | 0.004 |
| TRKB | 6.809241 | 0.004 |
| TXK | 3.26606 | 0.004 |
| Smad 4 | 14.28682 | 0.009 |
| FGFR1 | 6.441568 | 0.009 |
| Kallikrein 5 | 3.86346 | 0.009 |
| NM23-H1/H2 | 5.984695 | 0.009 |
| Plasminogen | 3.235235 | 0.009 |
| ROS | 3.08605 | 0.009 |
| S100A4 | 5.34344 | 0.009 |
| Serping 1 | 8.999197 | 0.009 |
| Serpin A8 | 8.339236 | 0.009 |
| SYK | 13.80922 | 0.009 |
| TRA-1-81 | 5.74969 | 0.009 |
| Trypsin 1 | 12.7068 | 0.009 |
| GDF11 | 59.09495 | 0.015 |
| Clusterin | 322.4728 | 0.015 |
| cTnT | 184.3612 | 0.015 |
| CutA | 217.5524 | 0.015 |
| Cyclin D1 | 27.38391 | 0.015 |
| Cystatin A | 9.459818 | 0.015 |
| Defensin | 227.0208 | 0.015 |
| Desmin | 348.2456 | 0.015 |
| DLL4 | 356.3274 | 0.015 |
| FGFR2 | 13.38653 | 0.015 |
| Fibrinogen | 1.47624 | 0.015 |
| Fibrinopeptide A | 2.450378 | 0.015 |
| Gas1 | 7.062109 | 0.015 |
| Gastrin | 229.4557 | 0.015 |
| GATA-3 | 226.9231 | 0.015 |
| Gelsolin | 19.97257 | 0.015 |
| Ghrelin | 222.7011 | 0.015 |
| GLP-1 | 230.2016 | 0.015 |
| HSP32 | 131.6209 | 0.015 |
| HSP60 | 31.54389 | 0.015 |
| HSP90 | 494.1763 | 0.015 |
| Omentin | 7.116758 | 0.015 |
| PIM2 | 7.22723 | 0.015 |
| S100A6 | 3.931869 | 0.015 |
| SCG3 | 4.290052 | 0.015 |
| Serotonin | 19.47403 | 0.015 |
| Serpin I1 | 90.50669 | 0.015 |
| SMAC | 34.58259 | 0.015 |
| TACE | 16.5547 | 0.015 |
| TAF4 | 19.69919 | 0.015 |
| Vitamin D-BP | 3.8112 | 0.015 |
| EDA-A2 | 1.933584 | 0.026 |
| GREMLIN | 7.798717 | 0.026 |
| ADAMTS-5 | 26.30608 | 0.026 |
| ADAMTS-L2 | 8.092354 | 0.026 |
| Adipsin | 4.670584 | 0.026 |
| COCO | 80.78475 | 0.026 |
| Complement | 12.27403 | 0.026 |
| factor H | | |
| Creatinine | 3.381759 | 0.026 |
| CSH1 | 19.40335 | 0.026 |
| Cystatin B | 4.660797 | 0.026 |
| DPPIV | 8.806647 | 0.026 |
| Endorphin Beta | 12.18614 | 0.026 |
| EphA3 | 4.353228 | 0.026 |
| ERRa | 8.560323 | 0.026 |
| EXTL2 | 9.640182 | 0.026 |
| FABP2 | 15.48814 | 0.026 |
| FoxP3 | 11.10388 | 0.026 |
| FRK | 6.539991 | 0.026 |
| GATA-4 | 8.792889 | 0.026 |
| GPBB | 4.819533 | 0.026 |
| GPI | 5.458149 | 0.026 |
| Hepcidin | 8.654806 | 0.026 |
| HSP40 | 24.74462 | 0.026 |
| HSP70 | 36.41347 | 0.026 |
| Midkine | 7.50768 | 0.026 |
| PD-1 | 4.574154 | 0.026 |
| PI 16 | 4.103296 | 0.026 |
| Pro-MMP-9 | 4.890788 | 0.026 |
| Pro-MMP-13 | 6.168079 | 0.026 |
| Prostasin | 3.562363 | 0.026 |
| PTPRD | 5.714606 | 0.026 |
| Ras | 2.219349 | 0.026 |
| RECK | 2.698033 | 0.026 |
| RYK | 14.21574 | 0.026 |
| SART1 | 4.866865 | 0.026 |
| SART3 | 7.807252 | 0.026 |
| SEMA3A | 21.36675 | 0.026 |
| Serpin A1 | 10.82167 | 0.026 |
| Serpin A5 | 10.12044 | 0.026 |
| Serpin A9 | 25.61264 | 0.026 |
| Serpin D1 | 39.69157 | 0.026 |
| SOX17 | 6.136459 | 0.026 |
| Survivin | 5.51442 | 0.026 |
| Syndecan-3 | 11.02041 | 0.026 |
| TFF1 | 13.13231 | 0.026 |
| TFF3 | 4.672818 | 0.026 |
| Thymidine | 6.306063 | 0.026 |
| Kinase-1 | | |
| TPA | 4.485392 | 0.026 |
| TPM1 | 4.696349 | 0.026 |
| Troponin I | 3.969662 | 0.026 |
| TRPC6 | 6.015505 | 0.026 |
| TRPM7 | 5.534693 | 0.026 |
| TSH | 6.897905 | 0.026 |
| VEGF R1 | 4.237943 | 0.026 |
| Vitamin | 2.602491 | 0.026 |
| K-dependent | | |
| protein S | | |
| Csk | 3.714331 | 0.041 |
| GDF5 | 7.403583 | 0.041 |
| GDF9 | 7.627866 | 0.041 |
| Latent | 2.175335 | 0.041 |
| TGF-beta bp1 | | |
| CRP | 2.274928 | 0.041 |
| DBI | 8.18511 | 0.041 |
| FABP3 | 6.118762 | 0.041 |
| Fetuin B | 3.836071 | 0.041 |
| HSP20 | 3.982229 | 0.041 |
| Itk | 5.91214 | 0.041 |
| ITM2B | 4.979435 | 0.041 |
| Kallikrein 6 | 4.252369 | 0.041 |
| Kallikrein 11 | 2.647301 | 0.041 |
| KCTD10 | 3.669272 | 0.041 |
| KLF4 | 5.015021 | 0.041 |
| PEPSINOGEN II | 4.863297 | 0.041 |
| PI 3Kinase | 4.839435 | 0.041 |
| p85 beta | | |
| PPARg2 | 3.64854 | 0.041 |
| Pro-Cathepsin B | 2.548219 | 0.041 |
| Progesterone | 6.519625 | 0.041 |
| ProSAAS | 2.417423 | 0.041 |
| Protein p65 | 4.260583 | 0.041 |
| PSA-total | 8.690313 | 0.041 |
| PYK2 | 3.968538 | 0.041 |
| PYY | 3.097189 | 0.041 |
| Resistin | 4.827942 | 0.041 |
| RET | 3.163772 | 0.041 |
| RIP1 | 5.863169 | 0.041 |
| Serpin A4 | 5.20463 | 0.041 |
| Serpin B5 | 7.07585 | 0.041 |
| SHBG | 6.730152 | 0.041 |
| SSTR5 | 3.193597 | 0.041 |
| Syndecan-1 | 5.401464 | 0.041 |
| Thrombin | 4.282239 | 0.041 |
| TIM-1 | 4.201384 | 0.041 |
| TOPORS | 4.280719 | 0.041 |
| TRPC1 | 10.01976 | 0.041 |
| Tyk2 | 6.504028 | 0.041 |
| TYRO10 | 4.131835 | 0.041 |
| Vasopressin | 4.743533 | 0.041 |
| VGF | 7.867878 | 0.041 |
| Visfatin | 4.710721 | 0.041 |

Table 7 shows that African samples demonstrated a minimum of a two fold increases over Asian samples in all 161 statistically significant proteins (determined by the Mann-Whitney U Test). This was the largest collection of statistically significant proteins in all three (3) relative comparisons.

Table 8 is a list (and directional indication relative to comparator) of Statistically Significant (≧0.05) proteins for Caucasian relative to Asian.

TABLE 8

| | | Mann-Whitney U test |
|---|---|---|
| Caucasian Relative | Fold | p < 0.05, significant |
| to Asian | Change | difference |
| Activin B | 233.5384 | 0.015 |
| Activin C | 710.1567 | 0.015 |
| Activin RIB/ALK-4 | 41.81165 | 0.015 |
| Activin RIIA | 652.6 | 0.015 |
| HSP60 | 44.54578 | 0.015 |
| HSP90 | 467.3104 | 0.015 |
| Activin RIA/ALK-2 | 30.44423 | 0.026 |
| BMP-3 | 11.79482 | 0.026 |
| CCR8 | 26.44067 | 0.026 |
| EDA-A2 | 2.279418 | 0.026 |
| ApoC3 | 13.22354 | 0.026 |
| Fen 1 | 4.761872 | 0.026 |
| GATA-4 | 8.507966 | 0.026 |
| HSP70 | 46.01337 | 0.026 |
| TRPM7 | 6.563931 | 0.026 |
| Activin RII A/B | 14.30805 | 0.041 |
| beta-Catenin | 8.715446 | 0.041 |
| MCP-1 | 4.242993 | 0.041 |
| ADAMTS-L2 | 10.49804 | 0.041 |
| FER | 43.20704 | 0.041 |
| FGFR1 | 8.320166 | 0.041 |
| HSP40 | 33.77603 | 0.041 |
| TAF4 | 15.76296 | 0.041 |
| Tec | 31.81704 | 0.041 |

Table 8 shows that Caucasian samples demonstrated a minimum of a twofold increases over Asian samples in all 24 statistically significant proteins (determined by the Mann-Whitney U Test). This was the second largest collection of statistically significant proteins in all three (3) relative comparisons.

Table 9 is a list (and directional indication relative to comparator) of Statistically Significant (≧0.05) proteins for Caucasian relative to African.

TABLE 9

| | | | Mann-Whitney U test |
|---|---|---|---|
| Caucasian Relative | | | p < 0.05, significant |
| to African | Fold Change | | difference |
| Presenilin 1 | 0.17695 | (−5.65131) | 0.026 |
| Presenilin 2 | 0.201391 | (−4.96546) | 0.026 |
| TSLP | 0.442966 | (−2.25750) | 0.026 |
| Vitamin D-BP | 0.28323 | (−3.53069) | 0.026 |
| Osteopontin | 0.262119 | (−3.81506) | 0.041 |

Table 9 shows that Caucasian samples demonstrated a minimum of a twofold decreases compared to African samples in all 5 statistically significant proteins (determined by the Mann-Whitney U Test). This was the smallest collection of statistically significant proteins in all three (3) relative comparisons.

The above data supports at least the following conclusions.

Biological Significance means greater than a 2-fold increase or decrease relative to comparator sample as written in the protocol definition. Biological Significance demonstrated 616/1000 proteins that met the criteria in at least one group. Also, 231/616 were common across all groups.

The data further showed that uniquely altered proteins (biologically significant in only one group):

Asian: 114/1000

African: 30/1000

Caucasian: 88/1000

A change in average production level is defined as greater than 25% increased/decreased relative to comparator per protocol definition. The data shows that the average production level changes demonstrated variations in all three groups in the shared 231 biologically significant proteins. In general, Asian sample or skin, relative to African and Caucasian samples or skin, showed decreased protein production levels, while Caucasian skin showed the most increase in production levels.

The statistical significance of the data using the Mann Whitney test further showed that the Asian skin demonstrated the largest number or amount of proteins, in the 95% confidence interval, relative to African skin. The second largest amount of proteins was Asian skin relative to Caucasian skin. The least amount of proteins was African skin relative to Caucasian skin.

The data supports the belief that a further analysis of the proteins and their pathways will improve the understanding of the differences in the structure, function, health and beauty of the skin amongst these ethnic groups.

The data confirms, as noted in the further studies below, that the mixture of two or more tissue cultures whether homogeneous or heterogeneous from the same sex is possible to achieve desired effect on a recipient. In other words, the blending of tissue cultures of one ethnic group, or amongst two or more ethnic groups, in a product, based on characteristics of the proteins in each ethnic group and desired characteristic(s) to be imparted to the skin of the recipient, is possible.

### Example II

Global gene expression (i.e. protein levels) by dermal fibroblasts between 3 ethnic groups, Caucasians (CA), African (AF) and Asian (AS) were compared. 6 patients from each ethnicity were recruited for this study. Dermal fibroblasts were isolated from each biopsy and grown under similar tissue culture conditions. Protein was extracted and samples underwent protein assay, testing and analysis according to in-house protocols. Data was further corrected and interpreted by BioFast Consultant Group as specified herein.

To evaluate protein expression in all samples, RayBio® Human Biotin Label Based Antibody Arrays—Human L-507 Array was utilized. This array is designed to evaluate 1000 proteins in duplicates. Two normalization steps were done to account for differences between the different arrays (intra array) or within each array (inter array). This includes normalization to the positive control and then background value is subtracted (negative control). Duplicates values were then averaged (this gives one final intensity value that is used for the final analysis and statistics).

From a biological perspective, it is expected that following data normalization, the average gene expression intensity will not differ between the different test groups (i.e. AS, AF and CA). Therefore, in order to test this, global intensity average of each sample (i.e. total of 18 samples) and ethnic group were calculated. Results of this initial analysis are shown in Table 10 below, shown as Table 10A 10B and 10C for pagination purposes.

TABLE 10A

| Prior to Data | | | | | | |
|---|---|---|---|---|---|---|
| Correction | AS1 | AS2 | AS3 | AS4 | AS5 | AS6 |
| average intensity | 413.2 | 597.1 | 343.8 | 893.2 | 718.5 | 736.2 |
| average per group | 617.0 | | | | | |
| SEM | 93.25 | | | | | |

TABLE 10B

| Prior to Data | | | | | | |
|---|---|---|---|---|---|---|
| Correction | AF1 | AF2 | AF3 | AF4 | AF5 | AF6 |
| average intensity | 984.2 | 1171.7 | 455.4 | 556.8 | 1060.4 | 1968.1 |
| average per group | 1032.8 | | | | | |
| SEM | 241.4 | | | | | |

TABLE 10C

| Prior to Data | | | | | | |
|---|---|---|---|---|---|---|
| Correction | CA1 | CA2 | CA3 | CA4 | CA5 | CA6 |
| average intensity | 1506.5 | 2324.8 | 380.3 | 358.6 | 3146.8 | 821.6 |
| average per group | 1423.1 | | | | | |
| SEM | 504.9 | | | | | |

Using the original normalized data values, fold changes for each gene was calculated among the different groups by performing 3 independent comparisons: AF/AS, CA/AS, CA/AF. Summarized results showing the overall number of statistically significant (ttest) up-regulated and down-regulated genes are shown in Table 11 below.

TABLE 11
Result Summary Prior to Data Correction

| | AF/AS | CA/AS | CA/AF |
|---|---|---|---|
| Total significantly upregulated | 259.0 | 94.0 | 7.0 |
| Total significantly downregulated | 1.0 | 0.0 | 13.0 |

It has been noted that many of the intensity (i.e. gene expression) values equaled “1”. This means that either the expression level of a given protein was below the lower detection limit of the assay (or lower than the negative control) or that an undetermined technical reason(s) caused the detection to fail for that particular protein. When all these “1's” are included in the analysis, they might provide false positive results. For example, if the average intensity of geneX for the Asian group is 1 and the average intensity of the same gene in African group is 300, data analysis will suggest a 300 fold up-regulation in the African group, where in fact the expression of geneX in the Asian group simply could not be determined (for unknown reasons).

From comparing the averages of the global intensity, it was noted that there were obvious differences in overall detection signal between the ethnic groups, with the lowest values observed in the Asian group and the highest in Caucasians. This means that on average “more protein” was detected in the Caucasian group compared with the other groups. This phenomenon is unexpected and is most likely a result of an artifact due to an unequal protein loading, non-consistent washing of the membranes, or other technical issues that evidently could not be corrected by the intra and inter array normalization steps.

Without wishing to be bound by a single theory, a series of issues were identified that could explain, at least partly, the unequal intensity detection levels between groups, as well as the abundance of undetermined detections. Protein loading in the arrays was not equal, some samples had very low to no detection, there is a saturation issue such that the intensity was very high to exceed the normal detection range of the scanner or to the point where it overlapped with another spot in the array, there is a background issue such that in some membranes it was noted that certain areas were darker (“dirty areas”) compared with the rest of the membrane.

In order to address the above issues and avoid drawing conclusions based on potentially false positive and negative results, the following steps were undertaken:

Data points that were deemed as artifacts were filtered out. Only intensity values that are larger than 100 but smaller than 50000 are considered valid. Therefore, all values smaller than 101 and higher than 49999 were filtered out/excluded. A summary of how many data points were “lost” due to this action is specified in the Table 12 shown below.

TABLE 12

| | AS 1 | AS 2 | AS 3 | AS 4 | AS 5 | AS 6 |
|---|---|---|---|---|---|---|
| Total removed samples | 668 | 675 | 916 | 420 | 504 | 553 |
| following data correction | | | | | | |
| Average of removed data | 623 | | | | | |
| point per group* | | | | | | |
| | AF 1 | AF 2 | AF 3 | AF 4 | AF 5 | AF 6 |
| Total removed samples | 266 | 374 | 865 | 542 | 246 | 190 |
| following data correction | | | | | | |
| Average of removed data | 414 | | | | | |
| point per group* | | | | | | |
| | CA 1 | CA 2 | CA 3 | CA 4 | CA 5 | CA 6 |
| Total removed samples | 114 | 186 | 826 | 688 | 59 | 794 |
| following data correction | | | | | | |
| Average of removed data | 344 | | | | | |
| point per group* | | | | | | |

To ensure that this change was sufficient in correcting the data, averages of global protein expression was compared between the groups. It was noted that filtering “bad” data points overall improved the data set, as the changes in global protein expression between the ethnic groups were mostly abolished. This is shown in the Table 13 below, shown as Tables 13A, 13B, and 13C for pagination purposes.

TABLE 13A

| Post Data | | | | | | |
|---|---|---|---|---|---|---|
| Correction | AS1 | AS2 | AS3 | AS4 | AS5 | AS6 |
| average intensity | 1027.7 | 1386.4 | 3400.3 | 1292.1 | 1215.8 | 1115.4 |
| average per group | 1572.9 | | | | | |
| SEM | 404.3 | | | | | |

TABLE 13B

| Post Data | | | | | | |
|---|---|---|---|---|---|---|
| Correction | AF1 | AF2 | AF3 | AF4 | AF5 | AF6 |
| average intensity | 1082.6 | 1324.2 | 2193.5 | 922.0 | 1063.4 | 2039.5 |
| average per group | 1437.5 | | | | | |
| SEM | 243.2 | | | | | |

TABLE 13C

| Post Data | | | | | | |
|---|---|---|---|---|---|---|
| Correction | CA1 | CA2 | CA3 | CA4 | CA5 | CA6 |
| average intensity | 1487.2 | 2245.1 | 1744.0 | 785.5 | 2371.5 | 2,390 |
| average per group | 1837.3 | | | | | |
| SEM | 282.5 | | | | | |

After DATA filtering (above), the intensity values of any given protein for any given group (AS, AF and CA) were further analyzed only if at least 3 of these values were available (n>=3 in a given group). For the entries/proteins respecting this parameter (n>=3), fold changes/ratios between the groups were calculated.

A summary of significantly up- and down-regulated genes after data correction is also shown in the Table 14 below.

TABLE 14

| | Results Summary After Data Correction | | |
|---|---|---|---|
| RATIO - ETHNICITY | AF/AS | CA/AS | CA/AF |
| TOTAL | 60 | 108 | 49 |
| Down | 0 | 1 | 6 |
| Up | 60 | 107 | 43 |

The significantly changed proteins were subsequently utilized for the study of protein network interactions, described below.

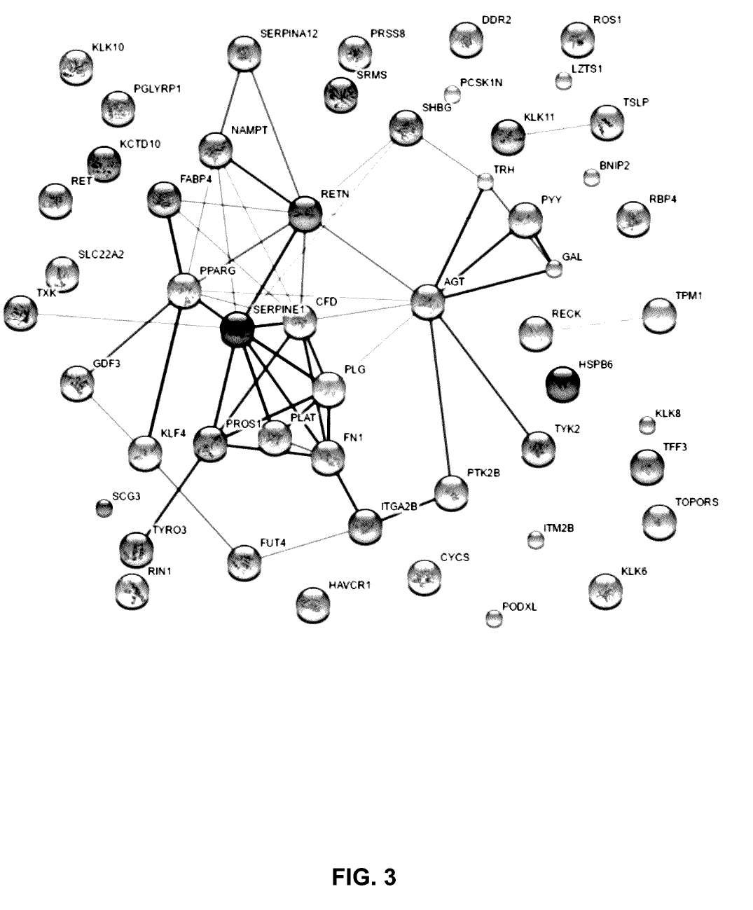

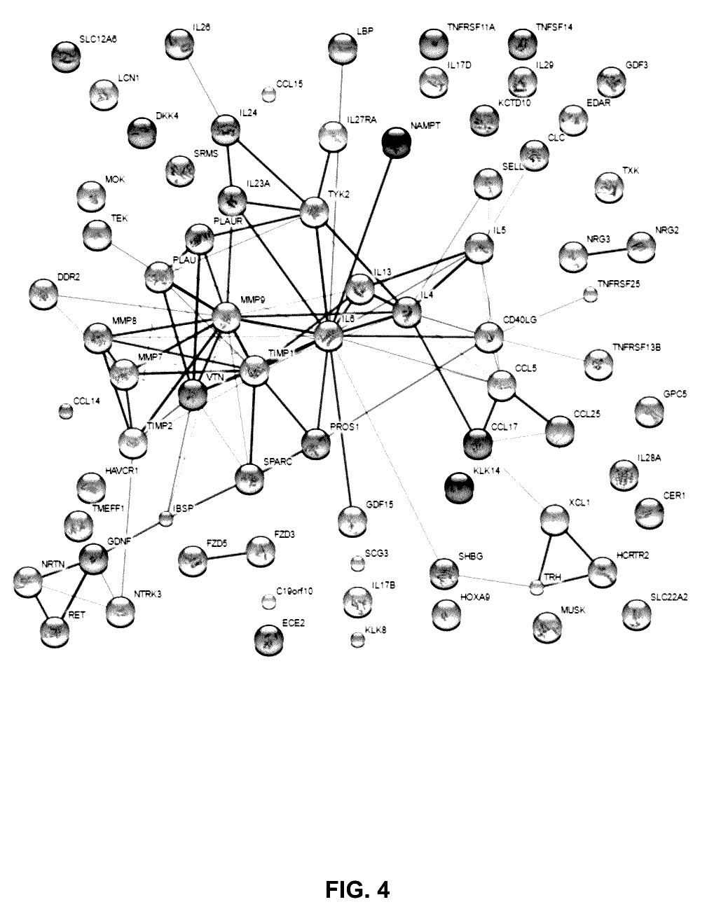

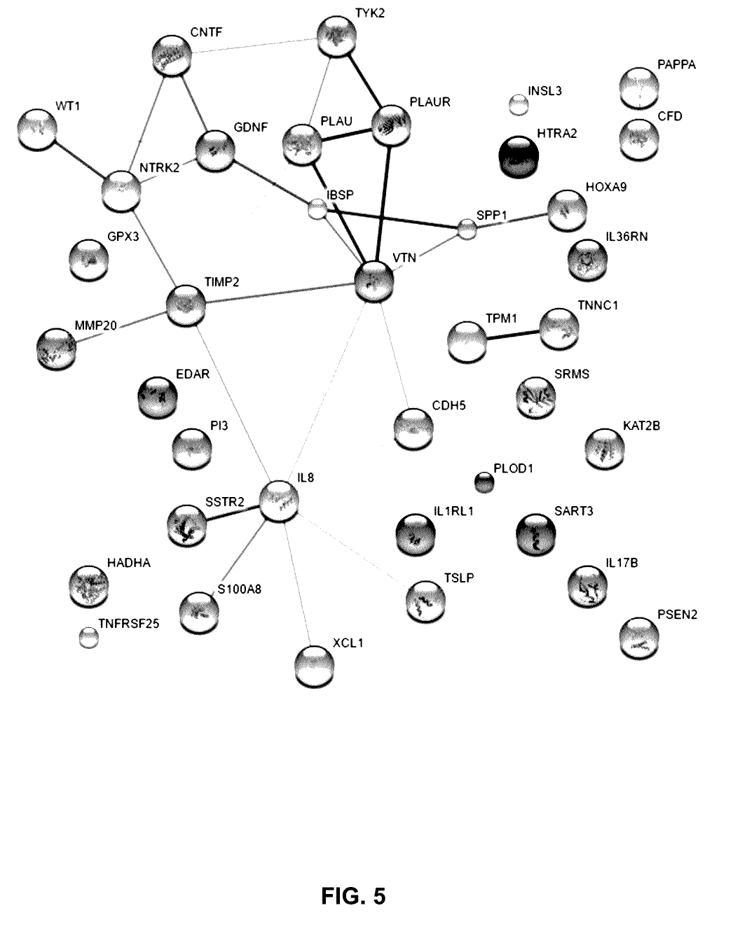

In order to identify the major Biological processes and cell signaling pathways that were differentially modulated from one ethnic group to another, data was subjected to a string network analysis. As input for the software, 3 separate lists of entries were used (as mentioned above), which included only the proteins that presented significant differences (p<0.05 by one tailed T-TEST) in expression levels between the groups (AF/AS; CA/AS; CA/AF. The overall protein networks for each of these 3 lists are shown in FIGS. 3 to 5.

FIGS. 3 to 5 show protein network AF/AS, CA/AS and CA/AF. The line thickness is proportional to the strength of data in support of functional interactions between protein

Out of the string network analysis, the first 50 (Top-50 ranked for statistical significance/false discovery rate) Biological processes were categorized/interpreted. Of note, processes that were considered generic/potentially pertinent to any biological process (e.g. “Cell Signaling”) were ignored.

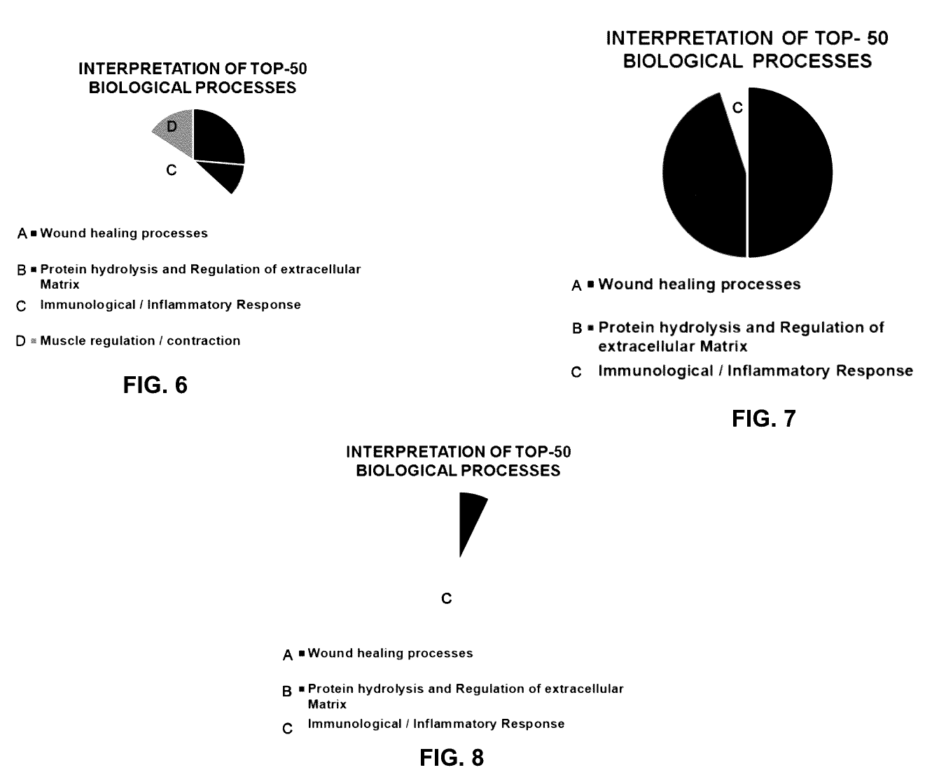

FIGS. 6 to 8 describe the main biological processes that are differentially modulated from one group to another are reported herein.

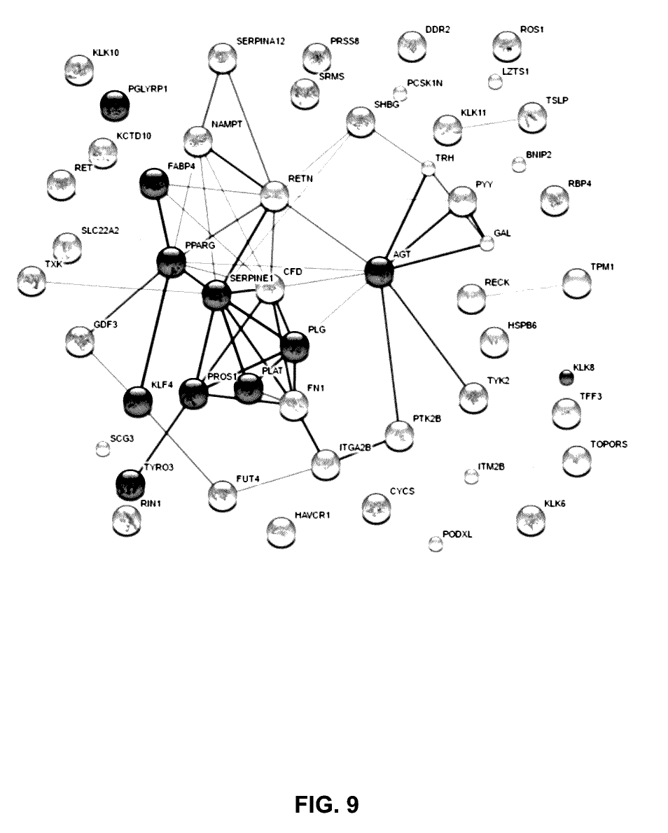

In addition, significant examples on how the top or best scoring biological processes fit within the overall protein networks are shown FIGS. 9 to 11.

In FIG. 9, it is apparent that most of the genes identified for wound healing processes (in red) do appear to be at the core of the strongest functional interactions of the network.

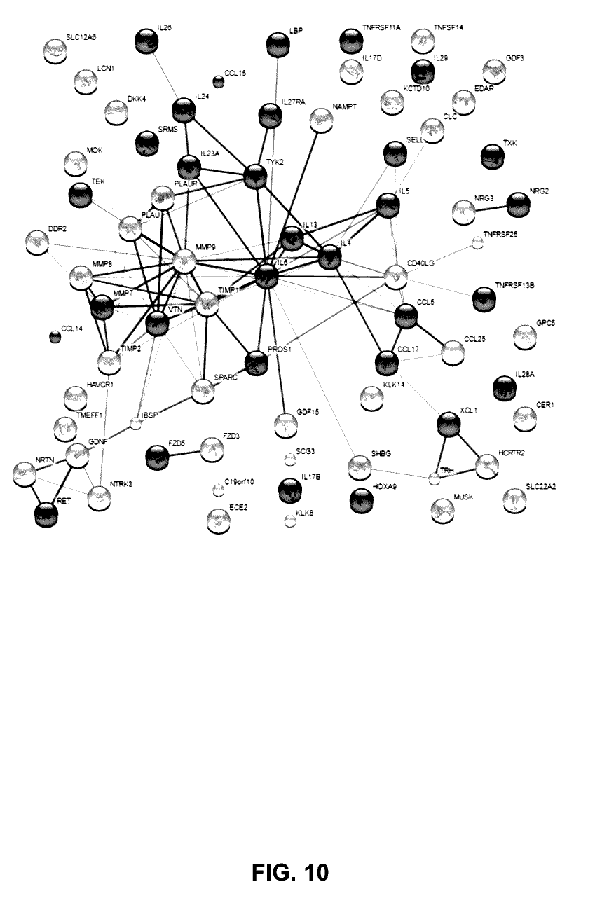

In FIG. 10, it is apparent that that most of the genes identified for immune response (in red) do appear to be functionally linked in the overall network.

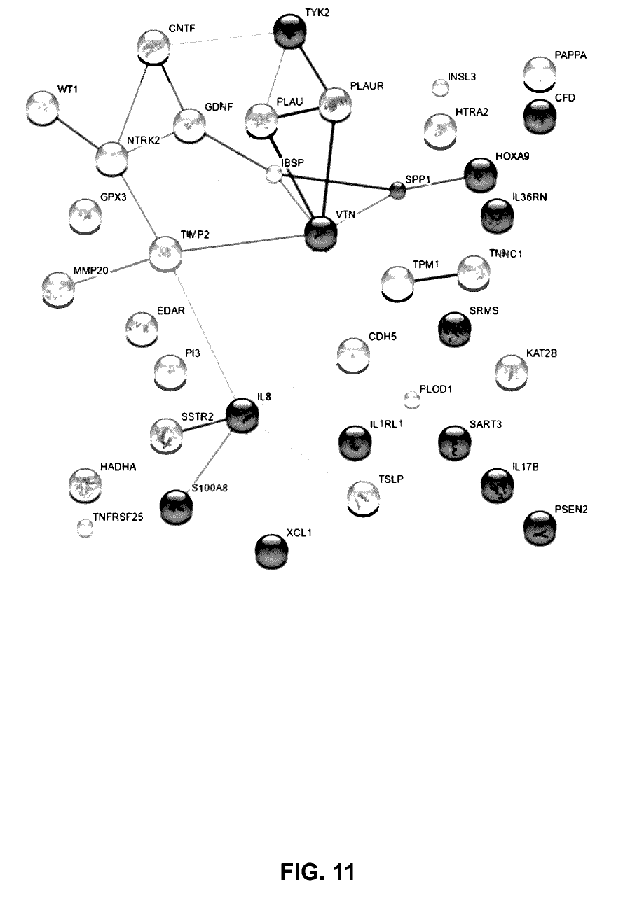

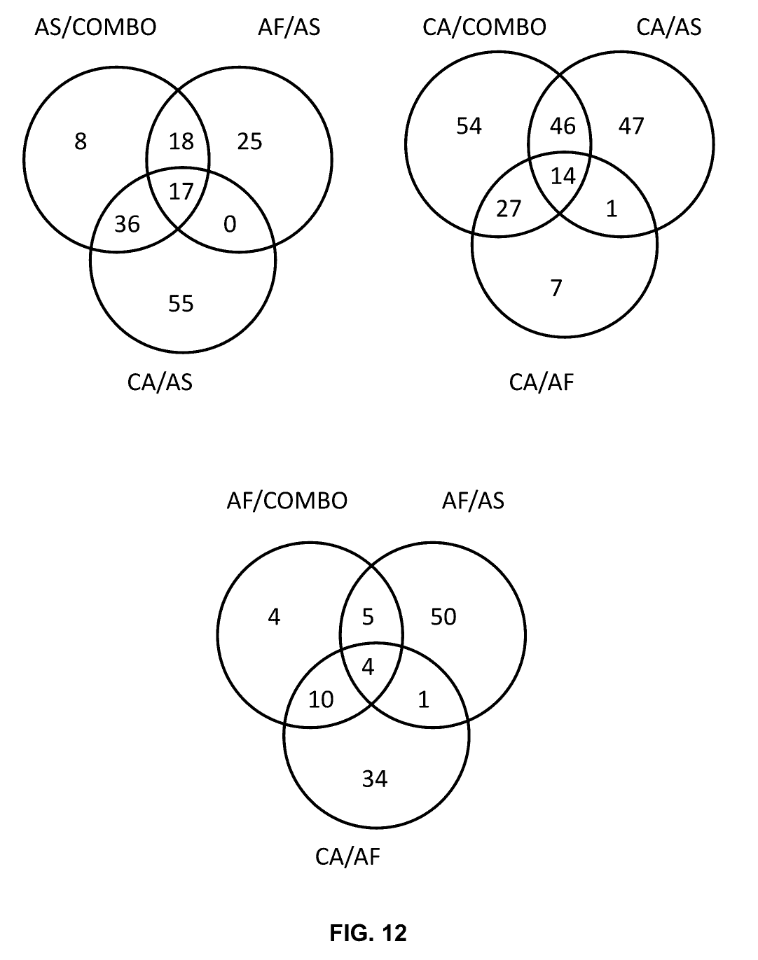

In FIG. 11 however, only 50% of the identified genes for immune response (in red) appear to be functionally linked in the overall network.

The most significant pathways fitting within the overall protein network for each of the 3 list of differentially modulated proteins, AF/AS, CA/AS and CA/AS, were “Complement and Coagulation Cascade”, “Cytokine-Cytokine Receptor interaction” and again “Cytokine-Cytokine Receptor interaction”, respectively.

Several conclusions can be drawn from the protein network analysis.

For AF/AS, the wound healing process, as well as the regulation of extracellular matrix, are the main biological processes that can distinguish AF from AS at molecular level. These processes are mostly up-regulated in AF compared to AS (as the majority of the proteins were up-regulated for that ratio). Notably, the “complement and coagulation pathway” seems to be at the core of the wound healing process.

For CA/AS, the immune system response/Inflammation is the main biological process at play, mostly up-regulated in CA, compared to AS (as the majority of the proteins were up-regulated for that ratio). Moreover, “cytokine-cytokine receptor pathway” and “JAK-STAT pathway” are likely involved in such differential basal modulation of the immune system between CA and AS.

For CA/AF, the immune response was again the top-scoring biological process, up-regulated in CA, compared to AF (as the majority of the proteins were up-regulated for that ratio). However, the same process did not strongly cover the core of the functional interactions of the overall STRING network (of differentially expressed proteins). Perhaps, in this case (CA/AF) there is a balance of several different processes at play, which we cannot clearly infer from the protein list and subsequent Network analysis. At any rate, a mixture of “cytokine-cytokine receptor pathway” and “complement and coagulation cascade” seem to cover the main differences between CA and AF.

Next, skin-relevant genes in each ethnic group were identified as follows. When values from at least 3 subjects (n=>3) of a given ethnicity/group were available, these values were averaged. The averages were compared to the value averages of the rest of the ethnicities, contingently that there was also availability of at least 3 values from the rest of the ethnicities. Significant differences between groups were tested by T-TEST (p<0.05 is considered statistically significant).

From the above, several conclusions are possible. CA presents mostly up-regulated genes, compared to the other ethnicities. AS presents mostly down-regulated genes, compared to the other ethnicities. AF presents equally UP- and DOWN-regulations, compared to other ethnicities. See Table 15.

TABLE 15

| | p < 0.05 | p < 0.05 | p < 0.05 |
|---|---|---|---|
| RATIO | AS/(AF + CA) | AF/(AS + CA) | CA/(AS + AF) |
| TOTAL | 79 | 23 | 141 |
| Down | 78 | 11 | 3 |
| Up | 1 | 12 | 138 |

In order to select/shortlist target genes that are differentially expressed/modulated in a specific group, while, at the same time, reducing the chance of selecting false positive, an approach was taken that favors the identification of genes that are differentially regulated in a particular group no matter what the control population is. Seventeen (17) genes were identified for the AS, fourteen (14) for the CA and four (4) for the AF group, as shown in the Venn diagrams of FIG. 12.

The following Tables 16, 17 and 18 summarize the findings discussed in the preceding paragraph.

TABLE 16
AS

| Gene | Fold | | Mass | | |
|---|---|---|---|---|---|
| name | change | p-Value | (kDa) | Function | Relevance to Skin |
| SRMS | 0.169108 | 0.04084 | 54.5 | Non-receptor tyrosine-kinase | n/a |
| GDF3 | 0.216263 | 0.00761 | 41.4 | Negative regulator of | TGFb is a major pro-fibrotic |
| | | | | TGFb/BMP signaling | pathway in skin. Reduced |
| | | | | | expression of this protein may |
| | | | | | contribute to hypertrophic scars |
| | | | | | or keloids in the Asian population |
| SHBG | 0.238325 | 0.00815 | 43.8 | Complexes with sex | Sex hormones affect various |
| | | | | hormones | aspects of skin biology and |
| | | | | | pathology. may have a role in |
| | | | | | skin pigmentation and |
| | | | | | androgen actions in skin of |
| | | | | | Asians |
| Tyk2 | 0.239025 | 0.00402 | 133.6 | Tyrosine kinase enzyme | Tyk2 is a therapeutic target for |
| | | | | | psoriasis-like skin |
| | | | | | inflammation. TYK2 deficiency |
| | | | | | is associated with skin bacterial |
| | | | | | infections which may suggest a |
| | | | | | role in barrier function |
| Creatinine | 0.262378 | 0.02641 | n/a | Byproduct of muscle cell | n/a |
| | | | | metabolism | |
| TSH | 0.271665 | 0.01403 | 28 | Thyroid stimulating hormone | TSH and the TSH receptor |
| | | | | | involved in skin keratinocyte |
| | | | | | and fibroblast growth; Reduced |
| | | | | | TSH levels associated with hair |
| | | | | | loss and dry rough pale skin. |
| TXK | 0.287523 | 0.03243 | 61.2 | Tyrosine kinase enzyme | Overexpression is observed in |
| | | | | | Behcet's disease |
| TIM-1 | 0.297133 | 0.00795 | 38.7 | membrane receptor | May be involved in modulation |
| | | | | | of allergic diseases such as |
| | | | | | asthma, allergic rhinitis and |
| | | | | | atopic dermatitis. |
| RET | 0.303647 | 0.00297 | 124.3 | Cell surface receptor tyrosine | Overexpression was found to |
| | | | | kinase | induce melanoma; mutations |
| | | | | | leading to hyperactivation is |
| | | | | | associated with cutaneous |
| | | | | | lichen amyloidosis. |
| Visfatin | 0.30562 | 0.00908 | 55.5 | Enzyme | Contribute to resolution of skin |
| | | | | | sclerosis; has direct anti-fibrotic |
| | | | | | effects; May be relevant to |
| | | | | | hypertrophic scars/keloids in |
| | | | | | Asians. May be also involved in |
| | | | | | inflammatory responses in psoriasis. |
| TYRO10 | 0.320326 | 0.00904 | 96.7 | Tyrosine kinase receptor for | Regulate remodelling of the |
| | | | | collagen | extracellular matrix and |
| | | | | | promote fibroblast migration |
| | | | | | during wound healing. |
| RELM alpha | 0.322593 | 0.00424 | 11.4 | Also known as FIZZ- 1 | Small secreted molecule |
| | | | | | associated with dermal fibrosis |
| | | | | | via its role in the induction of |
| | | | | | dermal adipocytes to myfibroblasts. |
| PSA-Free | 0.36243 | 0.03103 | 34 | Prostate specific antigen; | n/a |
| | | | | klk3 | |
| SCG3 | 0.369737 | 0.01215 | 53 | Secretogranin III | n/a |
| KCTD10 | 0.378113 | 0.02082 | 35.4 | Potassium Channel | n/a |
| | | | | Tetramerization Domain | |
| | | | | Containing 10 | |
| Vitamin | 0.447402 | 0.01595 | 75.1 | Anticoagulant function | Topical of Vitamin K1 |
| K-dependent | | | | | suppresses pigmentation and |
| protein S | | | | | improves wound healing |

TABLE 17
CA

| Gene | Fold | | Mass | | |
|---|---|---|---|---|---|
| name | change | p-Value | (kDa) | Function | Relevance to Skin |
| EDAR | 1.797218 | 0.01252 | 48.6 | TNFR superfamily member | EDAR agonists may improve |
| | | | | | skin dryness and eczema |
| Pro-MMP-7 | 2.130663 | 0.00299 | 29.7 | Matrix metallo Protease | Degrades ECM components. |
| | | | | | May play a role in skin aging. |
| | | | | | May be added to products |
| | | | | | designed to treat HS/keloids. It |
| | | | | | may contribute to remodelling |
| | | | | | of elastotic areas in |
| | | | | | sundamaged skin |
| SSEA-4 | 2.280554 | 0.01109 | n/a | Stage-specific embryonic | n/a |
| | | | | antigen | |
| MMP-10 | 2.3408 | 0.004 | 54.1 | Matrix metallo Protease | It might have a role in |
| | | | | | controlling scar formation |
| | | | | | during wound repair. Thus, it |
| | | | | | might be useful to control |
| | | | | | fibrosis and scan formation. |
| Vitronectin | 2.423404 | 0.00089 | 54.3 | Glycoprotein promoting cell | Regulate extra cellular matrix |
| | | | | adhesion via integrins | proteolysis and cell migration |
| | | | | | via stabilization of PAI1. |
| TWEAK R/ | 2.65139 | 0.0026 | 45.4 | TNFR superfamily member | The expression is altered in |
| TNFRSF12 | | | | | eczema skin, thus providing an |
| | | | | | antagonist might be beneficial |
| | | | | | in eczema. |
| uPA | 2.924766 | 0.00111 | 48.5 | Urokinase/serine protease | The absence of uPA signaling |
| | | | | | might be associated with the |
| | | | | | progression of dermal fibrosis |
| | | | | | has been shown to increase |
| | | | | | fibroblast proliferation and |
| | | | | | stimulate fibronectin secretion. |
| | | | | | May reduce wrinkles in Asians. |
| uPAR | 2.969332 | 0.00025 | 37 | Urokinase receptor | The absence of uPAR might be |
| | | | | | associated with the progression |
| | | | | | of dermal fibrosis |
| GDNF | 3.011637 | 0.00189 | 23.7 | Signaling molecule | It decreases with age. Thus |
| | | | | (promotes cell survival | providing it (at least in CA) |
| | | | | in neurons) | might prevent aging marks, |
| | | | | | including wrinkles |
| IL-17B | 3.071995 | 0.00327 | 20.4 | Pro-inflammatory cytokine | It might enhance skin |
| | | | | | inflammatory response |
| VCAM-1 | 3.145728 | 0.00103 | 81.3 | Adhesion molecule | It might increase susceptibility |
| (CD106) | | | | | to atopic dermitisis |
| Lymphotactin/ | 3.313039 | 0.00104 | 12.5 | Chemokine | Proinflammatory mediator that |
| XCL1 | | | | | could be involved in skin |
| | | | | | inflammation. |
| SRMS | 4.053276 | 0.00219 | 54.5 | Non-receptor tyrosine-kinase | n/a |

TABLE 18
AF

| Gene | Fold | | Mass | | |
|---|---|---|---|---|---|
| name | change | p-Value | (kDa) | Function | Relevance to skin |
| TPM1 | 2.203069 | 0.01017 | 32.7 | Tropo- | Regulate skin migra- |
| | | | | myosin | tion during wound |
| | | | | 1 | healing |
| Tyk2 | 2.374957 | 0.00377 | 133.6 | Tyrosine | Tyk2 is a therapeutic |
| | | | | kinase | target for psoriasis- |
| | | | | enzyme | like skin inflammation. |
| | | | | | TYK2 deficiency is |
| | | | | | associated with skin |
| | | | | | bacterial infections |
| | | | | | which may suggest a |
| | | | | | role in barrier function |
| TSLP | 2.537861 | 0.00297 | 18.1 | Thymic | Upregulated in skin of |
| | | | | Stromal | systemic sclerosis and |
| | | | | Lympho- | promote fibrosis, may |
| | | | | poietin | explain why individ- |
| | | | | | uals of African de- |
| | | | | | scent are more sus- |
| | | | | | ceptible to keloids. |
| Adipsin | 3.027837 | 0.00189 | 27 | Serine | n/a |
| | | | | peptidase | |
| | | | | (enzyme) | |

The analysis described above revealed that the number of genes that were differentially modulated in AS, AF and CA when compared to the rest of the groups (combined) were 79, 23 and 141, respectively. Further comparative analysis identified various genes that were found to be differentially regulated in each one of the tested group independently of the control group (as shown in the Venn diagram). Seventeen (17) genes were identified for the AS, fourteen (14) for the CA and four (4) for the AF group. Based on this data, it can be concluded that various genes (expressed by dermal fibroblasts) that control different aspects of skin function and pathology are differentially regulated in African, Asian and Caucasian skin. Thus, this data supports the proposition that differential gene regulation plays a role in racial differences of skin properties, appearance or pathology.

Furthermore, the data of the present disclosure proves and enables the rational for developing cosmetic and therapeutic products specifically suited for Asian, African or Caucasian (or other ethnic group or subgroup) skin based on proteins derived from the gene cells. Moreover, this data proves that the proteins can be selected from an ethnic group (or subgroup) in order to derive the benefit or characteristic desired of the product that can be used by any recipient.

Thus, the present disclosure has unexpectedly found that each ethnic group (and/or subgroup) has genes that are differentially regulated so that formulation of a targeted product that achieves maximization of a desired benefit or characteristic is now possible. Further, the targeted product can also be formulated with a higher proportionality of proteins in the overall product composition based on the desired benefits or characteristics of the product. This “tailoring” can be achieved by a single donor in a single ethnic group (or subgroup), as well as two or more donors in a single ethnic group or subgroup, and also in two or more donors in two or more groups and/or subgroups.

Table 19 that follows is a full list of non-redundant genes determined by the present disclosure.

FULL LIST OF NON-REDUNDANT GENES

| | | | |
|---|---|---|---|
| SRMS | Follistatin | SPINK1 | Neurokinin-A |
| IL-13 | Kremen-2 | EDAR | Beta IG-H3 |
| Musk | MMP-7 | NPTXR | CNTF |
| SHBG | IL-24 | SART3 | Troponin C |
| IL-27 | IL-23 | IL-7 | Mammaglobin A |
| Lipocalin-1 | PPARg2 | S-100b | Serpin A1 |
| CCR5 | IL-1 R8 | CFHR2 | LRP-1 |
| Creatinine | Pro-MMP-7 | XIAP | L-Selectin |
| | | | (CD62L) |
| NRG2 | TOPORS | Wilms | PYK2 |
| | | Tumor 1 | |
| IL-29 | BNIP2 | Troponin I | TWEAK R/ |
| | | | TNFRSF12 |
| Dkk-4 | Vitamin | MMP-8 | IL-26 |
| | K-dependent | | |
| | protein S | | |
| Resistin | MIP 2 | SART1 | IL-15 R alpha |
| TXK | Kallikrein 14 | GFR alpha-2 | VE-Cadherin |
| Dtk | IL-12 R beta 2 | PTN | TMEFF1/ |
| | | | Tomoregulin-1 |
| IL-28A | ProSAAS | CD 163 | Itk |
| XEDAR | TIMP-4 | Orexin B | S100A8 |
| Plasminogen | Kallikrein 8 | MBL | NRG3 |
| TIM-1 | Latent | sFRP-4 | GFR alpha-4 |
| | TGF-beta bp1 | | |
| RET | MMP-1 | FIH | VEGF R2 |
| | | | (KDR) |
| CLC | GDF3 | MMP-11/ | Pro-MMP-13 |
| | | Stromelysin-3 | |
| KCC3 | Tyk2 | TECK/CCL25 | IL-31 |
| TYRO10 | TSH | Insulysin/ | uPAR |
| | | IDE | |
| RELM alpha | TSLP | S100A6 | Livin |
| Cytokeratin 8 | Visfatin | OSM | VCAM-1 |
| | | | (CD106) |
| Csk | Adipsin | NRG1 Isoform | D-Dimer |
| | | GGF2 | |
| Kallikrein 6 | VDUP-1 | VEGF R3 | IL-17B R |
| RBP4 | TPA | MMP-9 | IL-17C |
| Frizzled-5 | HADHA | NM23-H1/H2 | IL-17D |
| LIGHT/ | IL-8 | Pro-MMP-9 | Hck |
| TNFSF14 | | | |
| Frizzled-3 | IGFBP-5 | NOV/CCN3 | Galanin |
| LIF R alpha | IL-33 | IL-17R | Lymphotactin/ |
| | | | XCL1 |
| IL-22 BP | IL-23p19 | Trappin-2 | HTRA2 |
| RANK/ | IL36RN | A2M | SSTR2 |
| TNFRSF11A | | | |
| GDNF | LH | Kallikrein 7 | FRK |
| Frizzled-4 | IL-11 | SSEA-4 | PTPRD |
| IL-17B | PCAF | Kininostatin/ | MMP-20 |
| | | kininogen | |
| PSA-Free | IL-18 R | VEGF-C | MIP-3 alpha |
| | beta/AcPL | | |
| MIG | INSL3 | GRP75 | Alpha 1 |
| | | | Microglobulin |
| HSP20 | VEGF R1 | TRKB | IL-31 RA |
| uPA | Vasopressin | ADAMTS-10 | GPX3 |
| SCG3 | VIP Receptor 2 | Tarc | IBSP |
| MIP-1d | TPM1 | MMP-10 | IL-1 R9 |
| IL-5 | IL-1 F9/IL-1 H1 | PARK7 | Endothelin |
| Prostasin | Presenilin 2 | GDF-15 | pro-Glucagon |
| KCTD10 | Transferrin | Vitronectin | GADD45A |
| FGF R4 | Thrombin | IL-6 | Omentin |
| IL-12 p70 | Kallikrein 11 | HSPA8 | |
| Fen 1 | IL-19 | Serpin A12 | |
| NeuroD1 | Prohibitin | IL-17RC | |
| FGF Basic | TIMP-1 | PAI-1 | |
| Cerberus 1 | SPARC | TCCR/WSX-1 | |
| NGF R | TMEFF2 | BMP-5 | |

Table 20 below contains information on each gene/protein that found to be differentially regulated in any group as compared with the average of the other two groups.

TABLE 20

| | | Accession | | Mass | |
|---|---|---|---|---|---|
| # | Protein symbol | Number | Full Name | (kDa) | Function |
| 1 | SRMS | Q9H3Y6 | Src-Related Kinase Lacking C-Terminal | 54.5 | Non-receptor tyrosine-kinase |
| | | | Regulatory Tyrosine And N-Terminal | | |
| 2 | IL-13 | P35225 | Interleukin 13 | 12.6 | Secreted cytokine |
| 3 | Musk | O15146 | Muscle Associated Receptor Tyrosine | 97 | gene encodes a muscle-specific |
| | | | Kinase | | tyrosine kinase receptor |
| 4 | SHBG | P04278 | Sex Hormone Binding Globulin | 43.8 | a steroid binding protein |
| 5 | IL-27 | Q8NEV9 | Interleukin 27 | 27.4 | heterodimeric cytokine which |
| | | | | | functions in innate immunity |
| 6 | Lipocalin-1 | P31025 | Lipocalin-1 | 19.2 | Small secretory extracellular |
| | | | | | transport protein that bind |
| | | | | | hydrophobic ligands |
| 7 | CCR5 | P51681 | C-C Motif Chemokine Receptor 5 | 40.5 | a member of the beta chemokine |
| | | | | | receptor family, which is predicted |
| | | | | | to be a seven transmembrane |
| | | | | | protein similar to G protein- |
| | | | | | coupled receptors |
| 8 | Creatinine | N/A | | | Byproduct of muscle cell metabolism |
| 9 | NRG2 | O14511 | Neuregulin 2 | 91.6 | member of the neuregulin family of |
| | | | | | growth and differentiation factors |
| 10 | IL-29 | Q8IU54 | Interferon, Lambda 1; Interleukin-29 | 21.8 | Cytokine with antiviral, antitumour |
| | | | | | and immunomodulatory activities. |
| 11 | Dkk-4 | Q9UBT3 | Dickkopf WNT Signaling Pathway | 24.8 | secreted protein; is involved in |
| | | | Inhibitor 4 | | embryonic development through |
| | | | | | its interactions with the Wnt |
| | | | | | signaling pathway. |
| 12 | Resistin | Q9HD89 | Resistin; FIZZ3 | 11.4 | Secreted protein |
| 13 | TXK | P42681 | TXK Tyrosine Kinase; PTK4 | 61.2 | Enzyme with a tyrosine kinase activity |
| 14 | Dtk | Q06418 | TYRO3 Protein Tyrosine Kinase | 96.9 | transmembrane receptor kinase receptor |
| 15 | IL-28A | Q8IZJ0 | Interleukin-28A; Interferon, Lambda 2; | 22.2 | Cytokine with antiviral, antitumour |
| | | | INFL2 | | &immunomodulatory activities. |
| | | | | | Plays a critical role in the antiviral |
| | | | | | defense, predominantly in epithelial |
| | | | | | tissues |
| 16 | XEDAR | Q9HAV5 | Ectodysplasin A2 Receptor; EDA-A2 | 32.7 | type III transmembrane protein of |
| | | | Receptor | | the TNFR (tumor necrosis factor |
| | | | | | receptor) superfamily |
| 17 | Plasminogen | P00747 | Plasminogen, PLG gene | 90 | Protein encoded by this gene is a |
| | | | | | secreted blood zymogen that is |
| | | | | | activated by proteolysis and converted |
| | | | | | to plasmin and angiostatin. |
| 18 | TIM-1 | Q96D42 | Hepatitis A Virus Cellular | 38.7 | Membrane receptor |
| | | | Receptor 1 | | |
| 19 | RET | P07949 | Ret Proto-oncogene | 124.3 | Receptor tyrosine kinase |
| 20 | CLC | Q05315 | Charcot-Leyden Crystal Galectin | 164.5 | Enzyme that regulates immune responses |
| 21 | KCC3 | Q9UHW9 | Solute Carrier Family 12 Member 6 | 127.6 | Member of the K—Cl cotransporter |
| | | | | | (KCC) family. |
| 22 | TYRO10 | Q16832 | Discoidin Domain Receptor Tyrosine | 96.7 | Tyrosine kinase that functions as |
| | | | Kinase 2 | | cell surface receptor for fibrillar |
| | | | | | collagen and regulates cell |
| | | | | | differentiation, remodeling of the |
| | | | | | extracellular matrix, cell migration |
| | | | | | and cell proliferation. |
| 23 | RELM alpha | Q9EP95 | Resistin-like molecule (Relm) alpha | 11.9 | Secreted protein and a hallmark |
| | | | | | signature gene for alternatively |
| | | | | | activated macrophages |
| 24 | Cytokeratin 8 | P05787 | Cytokeratin 8 | 53.7 | Member of the type II keratin family |
| 25 | Csk | P41240 | C-Src Tyrosine Kinase | 50.7 | Enzyme |
| 26 | Kallikrein 6 | Q92876 | Kallikrein Related Peptidase 6 | 26.8 | kallikrein subfamily of the |
| | | | | | peptidase S1 family of serine |
| | | | | | proteases (enzyme) |
| 27 | RBP4 | P02753 | Retinol Binding Protein 4 | 23 | Belongs to the lipocalin family and |
| | | | | | is the specific carrier for retinol |
| | | | | | (vitamin A alcohol) in the blood |
| 28 | Frizzled-5 | Q13467 | Frizzled Class Receptor 5 | 64.5 | Transmembrane receptor involved |
| | | | | | in Wnt signaling |
| 29 | LIGHT/ | O43557 | Tumor Necrosis Factor Superfamily | 26.35 | ligand for TNFRSF14, which is a |
| | TNFSF14 | | Member 14 | | member of the tumor necrosis |
| | | | | | factor receptor superfamily |
| 30 | Frizzled-3 | Q9NPG1 | Frizzled Class Receptor 3 | 76 | Transmembrane protein receptor |
| | | | | | for the wingless type MMTV |
| | | | | | integration site family of signaling |
| | | | | | proteins. |
| 31 | LIF R alpha | P15018 | Leukemia Inhibitory Factor | 22 | involved in the induction of |
| | | | | | hematopoietic differentiation in |
| | | | | | normal and myeloid leukemia cells |
| 32 | IL-22 BP | Q9GZX6 | Interleukin 22 | 20 | Cytokine that contributes to the |
| | | | | | inflammatory response in vivo. |
| 33 | RANK/ | Q9Y6Q6 | Tumor Necrosis Factor Receptor | 66 | Receptor for TNFSF11/RANKL/ |
| | TNFRSF11A | | Superfamily Member | | TRANCE/OPGL |
| 34 | GDNF | P39905 | Glial Cell Derived Neurotrophic | 24 | Neurotrophic factor that enhances |
| | | | Factor | | survival and morphological |
| | | | | | differentiation of dopaminergic |
| | | | | | neurons |
| 35 | Frizzled-4 | Q9ULV1 | Frizzled Class Receptor 4 | 60 | Transmembrane receptor coupled |
| | | | | | to the beta-catenin (CTNNB1) |
| | | | | | canonical signalling pathway. |
| 36 | IL-17B | Q9UHF5 | Interleukn 17B | 20 | Stimulates the release of the |
| | | | | | proinflammatory cytokines tumor |
| | | | | | necrosis factor alpha and IL-1- |
| | | | | | beta from the monocytic cell line |
| | | | | | THP-1. |
| 37 | PSA-Free | P07288 | Kallikrein Related Peptidase 3 | 28 | Hydrolyzes semenogelin-1 thus |
| | | | | | leading to the liquefaction of the |
| | | | | | seminal coagulum. |
| 38 | MIG | Q07325 | Chemokine (C-X-C Motif) Ligand 9 | 14 | Cytokine that affects the growth, |
| | | | | | movement, or activation state of |
| | | | | | cells that participate in immune |
| | | | | | and inflammatory response. |
| | | | | | Chemotactic for activated T-cells. |
| 39 | HSP20 | O14558 | Heat Shock Protein Family | 17 | Encodes a heat shock protein |
| 40 | uPA | P00749 | Plasminogen Activator, Urokinase | 48.5 | Specifically cleaves the zymogen |
| | | | | | plasminogen to form the active |
| | | | | | enzyme plasmin. |
| 41 | SCG3 | Q8WXD2 | Secretogranin III | 53 | A member of the |
| | | | | | chromogranin/secretogranin family |
| | | | | | of neuroendocrine secretory proteins. |
| 42 | MIP-1d | Q16663 | Macrophage Inflammatory Protein 5 | 12 | Chemotactic factor that attracts T- |
| | | | | | cells and monocytes. |
| 43 | IL-5 | P05113 | Interleukin 5 | 15 | Signaling compounds that are |
| | | | | | mediators of the immune |
| | | | | | response. They control many |
| | | | | | different cellular functions |
| | | | | | including proliferation, |
| | | | | | differentiation and cell |
| | | | | | survival/apoptosis |
| 44 | Prostasin | Q16651 | Serine Protease 8 | 36 | Enzyme |
| 45 | KCTD10 | Q9H3F6 | Potassium Channel Tetramerization | 35 | May be involved in cell proliferation |
| | | | Domain Containing 10 | | |
| 46 | FGF R4 | P22455 | Fibroblast Growth Factor Receptor 4 | 87.9 | mediate angiogenesis, wound |
| | | | | | healing, cell migration, neural |
| | | | | | outgrowth and embryonic development. |
| 47 | IL-12 p70 | P29459 | Interleukin 12A | 24.8 | Cytokine that can act as a growth |
| | | | | | factor for activated T and natural |
| | | | | | killer cells and enhance the lytic |
| | | | | | activity of NK/lymphokine-activated |
| | | | | | Killer cells |
| 48 | Fen 1 | P39748 | Flap Structure-Specific Endonuclease 1 | 42.5 | The enzyme removes 5′ |
| | | | | | overhanging flaps in DNA repair |
| | | | | | and processes the 5′ ends of |
| | | | | | Okazaki fragments in lagging |
| | | | | | strand DNA synthesis. |
| 49 | NeuroD1 | Q13562 | Neuronal Differentiation 1 | 40 | This gene encodes a member of the |
| | | | | | NeuroD family of basic helix-loop- |
| | | | | | helix (bHLH) transcription factors. |
| 50 | FGF Basic | P09038 | Basic Fibroblast Growth Factor | 30 | Growth factor that stimulate cell |
| | | | | | growth of fibroblasts |
| 51 | Cerberus 1 | O95813 | Cerberus 1, DAN Family BMP | 30 | belongs to a group of bone |
| | | | Antagonist | | morphogenetic protein (BMP) |
| | | | | | antagonists. |
| 52 | NGF R | P08138 | Nerve Growth Factor receptor | 45 | Membrane-bound receptor for |
| | | | | | nerve growth factor |
| 53 | Follistatin | P19883 | Follistatin | 38 | single-chain gonadal protein that |
| | | | | | specifically inhibits follicle- |
| | | | | | stimulating hormone release. |
| 54 | Kremen-2 | Q8NCW0 | Kringle Containing Transmembrane | 49 | Receptor for Dickkopf protein. |
| | | | Protein 2 | | Cooperates with Dickkopf to block |
| | | | | | Wnt/beta-catenin signaling. |
| 55 | MMP-7 | P09237 | Matrix Metallopeptidase 7 | 29.6 | Enzyme that breaks down extracellular |
| | | | | | matrix |
| 56 | IL-24 | Q13007 | Interleukin 24 | 23.8 | Cytokine |
| 57 | IL-23 | Q9NPF7 | Interleukin 23 | 20.7 | heterodimeric cytokine which functions |
| | | | | | in innate and adaptive immunity. |
| 58 | PPARg2 | P37231 | Peroxisome Proliferator Activated | 57.6 | Nuclear receptor |
| | | | Receptor Gamma | | |
| 59 | IL-1 R8 | Q9NZN1 | Interleukin 1 Receptor Accessory | 79.9 | IL-1 receptor. |
| | | | Protein Like 1 | | |
| 60 | Pro-MMP-7 | | | | |
| 61 | TOPORS | Q9NS56 | TOP1 Binding Arginine/Serine Rich | 119 | ubiquitin-protein E3 ligase (enzyme). |
| | | | Protein | | |
| 62 | BNIP2 | Q12982 | BCL2/Adenovirus E1B 19 kDa Interacting | 36 | Intracellular protein |
| | | | Protein 2 | | |
| 63 | Vitamin | P07225 | Protein S (Alpha) | 75 | Anticoagulant plasma protein; It |
| | K-dependent | | | | helps to prevent coagulation and |
| | protein S | | | | stimulating fibrinolysis. |
| 64 | MIP 2 | P19875 | C-X-C Motif Chemokine Ligand 2 | 11 | Produced by activated monocytes |
| | | | | | and neutrophils and expressed at |
| | | | | | sites of inflammation. |
| 65 | Kallikrein 14 | Q9P0G3 | Kallikrein Related Peptidase 14 | 29 | enzyme |
| 66 | IL-12 R beta 2 | Q99665 | Interleukin 12 Receptor Subunit Beta 2 | 89 | Receptor for interleukin-12. |
| 67 | ProSAAS | Q9UHG2 | Proprotein Convertase Subtilisin/ | 27 | Has an endopeptidase inhibitor activity; |
| | | | Kexin Type 1 Inhibitor | | inhibitor of prohormone convertase 1 |
| 68 | TIMP-4 | Q99727 | TIMP Metallopeptidase Inhibitor 4 | 25 | Inhibitors of the matrix |
| | | | | | metalloproteinases, |
| 69 | Kallikrein 8 | O60259 | Kallikrein Related Peptidase 8 | 28 | Serine protease (enzyme) |
| 70 | Latent | Q14766 | Latent Transforming Growth Factor Beta | 186.7 | Involved in the assembly, |
| | TGF-beta bp1 | | Binding Protein 1 | | secretion and targeting of TGFB1 |
| | | | | | to action sites |
| 71 | MMP-1 | P03956 | Matrix Metallopeptidase 1 | 54 | Enzyme envolves in degradation |
| | | | | | of extracellular matrix |
| 72 | GDF3 | Q9NR23 | Growth Differentiation Factor 3 | 41.3 | Growth factor |
| 73 | Tyk2 | P29597 | Tyrosine Kinase 2 | 133.6 | Tyrosine kinase enzyme |
| 74 | TSH | P01222 | Thyroid Stimulating Hormone Beta | 15.6 | Hormone |
| 75 | TSLP | Q969D9 | Thymic Stromal Lymphopoietin | 18.1 | Cytokine |
| 76 | Visfatin | P43490 | Nicotinamide Phosphoribosyltransferase | 55.5 | Enzyme |
| 77 | Adipsin | P00746 | Complement Factor D (Adipsin) | 27 | Serine peptidase (enzyme) |
| 78 | VDUP-1 | Q9H3M7 | Thioredoxin Interacting Protein | 43.6 | Enzyme |
| 79 | TPA | P00750 | Plasminogen Activator, Tissue Type | 62.9 | Enzyme |
| 80 | HADHA | P40939 | Hydroxyacyl-CoA Dehydrogenase/ | 83 | An enzyme involved in Lipid |
| | | | 3-Ketoacyl-CoA Thiolase/Enoyl-CoA | | metabolism. |
| | | | Hydratase (Trifunctional Protein), | | |
| | | | Alpha Subunit | | |
| 81 | IL-8 | P10145 | Chemokine (C-X-C Motif) Ligand 8 | 11.1 | This chemokine is one of the |
| | | | | | major mediators of the |
| | | | | | inflammatory response |
| 82 | IGFBP-5 | P24593 | Insulin Like Growth Factor Binding | 30.5 | IGF-binding proteins prolong the |
| | | | Protein 5 | | half-life of the IGFs and mediate |
| | | | | | the growth promoting effects of |
| | | | | | IGFs. |
| 83 | IL-33 | O95760 | Interleukin 33 | 30.7 | Cytokine that binds to and signals |
| | | | | | through the IL1RL1/ST2 receptor |
| | | | | | which in turn activates NF-kappa- |
| | | | | | B and MAPK signaling pathways |
| | | | | | in target cells |
| 84 | IL-23p19 | Q9NPF7 | Interleukin 23 Subunit Alpha | 20.7 | Associates with IL12B to form |
| | | | | | the IL-23 interleukin, a |
| | | | | | heterodimeric cytokine which |
| | | | | | functions in innate and adaptive |
| | | | | | immunity. |
| 85 | IL36RN | Q9UBH0 | Interleukin 36 Receptor Antagonist | 16.9 | member of the interleukin 1 |
| | | | | | cytokine family with specific anti- |
| | | | | | inflammatory properties |
| 86 | LH | P22888 | Luteinizing Hormone/ | 78.6 | G protein coupled receptor type 1 |
| | | | Choriogonadotropin Receptor | | |
| 87 | IL-11 | P20809 | Interleukin 11 | 21.4 | Cytokine that stimulates the |
| | | | | | proliferation of various cells |
| 88 | PCAF | Q92831 | Lysine Acetyltransferase 2B | 93 | This enzyme associates with other |
| | | | | | factors to regulate gene transcription. |
| 89 | IL-18R | O95256 | Interleukin 18 Receptor Accessory | 68.3 | The protein encoded by this gene |
| | beta/AcPL | | Protein | | is an accessory subunit of the |
| | | | | | heterodimeric receptor for |
| | | | | | interleukin 18 (IL18), a |
| | | | | | proinflammatory cytokine involved |
| | | | | | in inducing cell-mediated immunity. |
| 90 | INSL3 | P51460 | Insulin Like 3 | 14.5 | member of the insulin-like |
| | | | | | hormone superfamily |
| 91 | VEGF R1 | P17948 | Vascular Permeability Factor Receptor; | 150.7 | Tyrosine-protein kinase receptor |
| | | | Tyrosine-Protein Kinase Receptor FLT; | | |
| | | | Fms Related Tyrosine Kinase 1 | | |
| 92 | Vasopressin | P01185 | Arginine Vasopressin; Prepro-Arginine- | 17.3 | Arginine vasopressin is a posterior |
| | | | Vasopressin-Neurophysin II; Antidiuretic | | pituitary hormone that is |
| | | | Hormone | | synthesized in the supraoptic |
| | | | | | nucleus and paraventricular |
| | | | | | nucleus of the hypothalamus. |
| 93 | VIP | P41587 | Vasoactive Intestinal Peptide Receptor 2; | 49.4 | Vasoactive intestinal peptide is |
| | Receptor 2 | | Pituitary Adenylate Cyclase-Activating | | involved in smooth muscle |
| | | | Polypeptide Type III Receptor | | relaxation |
| 94 | TPM1 | P09493 | Tropomyosin 1 (Alpha); | 32.7 | actin-binding proteins involved in |
| | | | Cardiomyopathy, Hypertrophic 3 | | the contractile system of striated |
| | | | | | and smooth muscles and the |
| | | | | | cytoskeleton of non-muscle cells. |
| 95 | IL-1 F9/ | Q9NZH8 | Interleukin 36, Gamma; Interleukin-1 | 18.7 | Belongs to IL1 cytokine family |
| | IL-1 H1 | | Homolog 1; Interleukin-1 Epsilon | | |
| 96 | Presenilin 2 | P49810 | Presenilin 2; Alzheimer Disease 4 | 50.1 | Presenilins are postulated to |
| | | | | | regulate ameloid precursor protein |
| | | | | | processing through their effects on |
| | | | | | gamma-secretase, an enzyme |
| | | | | | that cleaves APP. |
| 97 | Transferrin | P02787 | Transferrin; Beta-1 Metal-Binding | 77 | transport iron from the intestine, |
| | | | Globulin; Siderophilin | | reticuloendothelial system, and |
| | | | | | liver parenchymal cells to all |
| | | | | | proliferating cells in the body |
| 98 | Thrombin | P00734 | Coagulation Factor II (Thrombin); | 70 | Coagulation factor II is |
| | | | Prepro-Coagulation Factor II | | proteolytically cleaved to form |
| | | | | | thrombin in the first step of the |
| | | | | | coagulation cascade which |
| | | | | | ultimately results in the stemming |
| | | | | | of blood loss. have antimicrobial |
| | | | | | activity against E. coli and |
| | | | | | P. aeruginosa. |
| 99 | Kallikrein 11 | Q9UBX7 | Kallikrein Related Peptidase 11; | 31 | Kallikreins are a subgroup of |
| | | | Trypsin-Like Protease; | | serine proteases having diverse |
| | | | Serine Protease 20; Hippostasin | | physiological functions. Growing |
| | | | | | evidence suggests that many |
| | | | | | kallikreins are implicated in |
| | | | | | carcinogenesis and some have |
| | | | | | potential as novel cancer and |
| | | | | | other disease biomarkers. |
| 100 | IL-19 | Q9UHD0 | Interleukin 19 | 20.4 | cytokine that belongs to the IL10 |
| | | | | | cytokine subfamily found to be |
| | | | | | preferentially expressed in |
| | | | | | monocytes. It can lead to the |
| | | | | | activation of the signal transducer |
| | | | | | and activator of transcription 3 |
| | | | | | (STAT3). |
| 101 | Prohibitin | P35232 | Prohobitin | 29.8 | play a role in human cellular |
| | | | | | senescence and tumor |
| | | | | | suppression. localized in the inner |
| | | | | | membrane of mitochondria. |
| 102 | TIMP-1 | P01033 | Metalloproteinase inhibitor 1 | 23.2 | Irreversibly inactivates collagenases |
| 103 | SPARC | P09486 | Basement-membrane protein 40/ | 34.6 | regulates cell growth through |
| | | | Osteonectin | | interactions with the extracellular |
| | | | | | matrix and cytokines |
| 104 | TMEFF2 | Q9UIK5 | Transmembrane Protein With EGF-Like | 41.4 | Transmembrane protein |
| | | | And Two Follistatin-Like Domains 2 | | |
| 105 | SPINK1 | P00995 | Serine protease inhibitor Kazal-type 1 | 8.5 | Serine protease inhibitor which |
| | | | | | exhibits anti-trypsin activity |
| 106 | EDAR | Q9UNE0 | Tumor necrosis factor receptor | 48.6 | TNF Receptor superfamily member |
| | | | superfamily member EDAR | | |
| 107 | NPTXR | O95502 | Neuronal pentraxin receptor | 52.8 | Receptor |
| 108 | SART3 | Q15020 | Squamous cell carcinoma antigen | 109.9 | functions as a recycling factor of |
| | | | recognized by T-cells 3 | | the splicing machinery |
| 109 | IL-7 | P13232 | Interleukin-7 | 20.2 | Cytokine |
| 110 | S-100b | P04271 | Protein S100-B | 10.7 | Small protein that binds ions |
| 111 | CFHR2 | P36980 | Complement factor H-related protein 2 | 30.6 | Involved in complement regulation |
| 112 | XIAP | P98170 | E3 ubiquitin-protein ligase XIAP | 56.7 | Enzyme |
| 113 | Wilms | P19544 | Wilms tumor protein | 49.2 | Transcription factor that plays an |
| | Tumor 1 | | | | important role in cellular |
| | | | | | development and cell survival |
| 114 | Troponin I | P19237 | Troponin I, cardiac muscle | 24 | Troponin I is the inhibitory subunit |
| | | | | | of troponin, the thin filament |
| | | | | | regulatory complex which confers |
| | | | | | calcium-sensitivity to striated |
| | | | | | muscle actomyosin ATPase activity |
| 115 | MMP-8 | P22894 | Neutrophil collagenase | 53.4 | Enzyme; Can degrade fibrillary |
| | | | | | type I, II, and III collagens |
| 116 | SART1 | O43290 | Squamous Cell Carcinoma Antigen | 90.2 | Plays a role in mRNA splicing as a |
| | | | Recognized By T-Cells 1 | | component of the U4/U6-U5 tri-snRNP |
| 117 | GFR alpha-2 | O00451 | GDNF family receptor alpha-2 | 51.5 | Receptor for neurturin, canonically |
| 118 | PTN | P21246 | Pleiotrophin | 18.9 | growth factor that induces neurite |
| | | | | | outgrowth and which is mitogenic |
| | | | | | for fibroblasts, epithelial, and |
| | | | | | endothelial cells |
| 119 | CD 163 | Q86VB7 | Scavenger receptor cysteine-rich type 1 | 125.4 | Receptor that may protect from |
| | | | protein M130 | | oxidative damage |
| 120 | Orexin B | O43612 | Orexin (Hypocretin) | 13.4 | Neuropeptide |
| 121 | MBL | P11226 | mannose-binding lectin 2/Mannose-binding | 26.1 | Calcium-dependent lectin involved |
| | | | protein C | | in innate immune defense. |
| 122 | sFRP-4 | Q6FHJ7 | Secreted frizzled-related protein 4 | 39.8 | Soluble frizzled-related proteins |
| 123 | FIH | Q9NWT6 | Hypoxia-inducible factor 1-alpha inhibitor | 40.3 | Inhibitor of HIF1-alpha |
| 124 | MMP-11/ | P24347 | Stromelysin-3/Matrix metalloproteinase-11 | 54.6 | Matrix metallo proteinase |
| | Stromelysin-3 | | | | |
| 125 | TECK/CCL25 | O15444 | C-C motif chemokine 25 | 16.6 | Signalling chemokine, ligand of |
| | | | | | receptor CCR9 |
| 126 | Insulysin/ | P14735 | Insulin-degrading enzyme | 118 | Plays a role in the cellular |
| | IDE | | | | breakdown of insulin, IAPP, |
| | | | | | glucagon, bradykinin, kallidin and |
| | | | | | other peptides |
| 127 | S100A6 | P06703 | Protein S100-A6 | 10.2 | May function as calcium sensor |
| | | | | | and modulator, contributing to |
| | | | | | cellular calcium signaling |
| 128 | OSM | P13725 | Oncostatin-M | 28.5 | Growth regulator. Inhibits the |
| | | | | | proliferation of a number of tumor |
| | | | | | cell lines |
| 129 | NRG1 Isoform | Q02297 | Neuregulin 1 | 70.4 | Direct ligand for ERBB3 and |
| | GGF2 | | | | ERBB4 tyrosine kinase receptors. |
| | | | | | Concomitantly recruits ERBB1 |
| | | | | | and ERBB2 coreceptors |
| 130 | VEGF R3 | P35916 | Vascular endothelial growth factor | 152.7 | Tyrosine-protein kinase receptor |
| | | | receptor 3 | | |
| 131 | MMP-9 | P14780 | Matrix metalloproteinase-9 | 78.4 | May play a role in proteolysis of |
| | | | | | the extracellular matrix (Collagen |
| | | | | | type III degradation) |
| 132 | NM23-H1/H2 | P15531 | Nucleoside diphosphate kinase A | 17.1 | Major role in the synthesis of |
| | | | | | nucleoside triphosphates other |
| | | | | | than ATP |
| 133 | Pro-MMP-9 | P14780 | Pro-Matrix metalloproteinase-9 | 78.4 | May play a role in proteolysis of |
| | | | | | the extracellular matrix (Collagen |
| | | | | | type III degradation) |
| 134 | NOV/CCN3 | P48745 | Protein NOV homolog | 39.1 | playing a role in various cellular |
| | | | | | processes including proliferation, |
| | | | | | adhesion, migration, differentiation |
| | | | | | and survival. It binds to integrins |
| | | | | | and membrane receptors such as |
| | | | | | NOTCH1 |
| 135 | IL-17R | Q96F46 | Interleukin-17 receptor A | 96.1 | Pro-inflammatory cytokine receptor |
| 136 | Trappin-2 | P19957 | Elafin (Elastase-specific inhibitor) | 12.3 | Neutrophil and pancreatic |
| | | | | | elastase-specific inhibitor of skin. |
| | | | | | It may prevent elastase-mediated |
| | | | | | tissue proteolysis |
| 137 | A2M | P01023 | Alpha-2-macroglobulin | 163.3 | It Is able to inhibit all four classes |
| | | | | | of proteinases by a unique |
| | | | | | ‘trapping’ mechanism |
| 138 | Kallikrein 7 | P49862 | Kallikrein-7 | 27.5 | May catalyze the degradation of |
| | | | | | intercellular cohesive structures in |
| | | | | | the cornified layer of the skin in |
| | | | | | the continuous shedding of cells |
| | | | | | from the skin surface |
| 139 | SSEA-4 | n/a | Stage-specific embryonic antigen 4/ | n/a | n/a |
| 140 | Kininostatin/ | P01042 | Kininogen-1 | 72 | Kininogens are inhibitors of thiol |
| | kininogen | | | | proteases |
| 141 | VEGF-C | P49767 | Vascular endothelial growth factor C | 46.9 | Growth factor active in |
| | | | | | angiogenesis, and endothelial cell |
| | | | | | growth (ligand of VEGFR3) |
| 142 | GRP75 | P38646 | Heat Shock Protein Family A (Hsp70) | 73.7 | Implicated in the control of cell |
| | | | Member 9 | | proliferation and cellular aging. |
| | | | | | May also act as a chaperone for |
| | | | | | protein folding |
| 143 | TRKB | Q16288 | Neurotrophic Tyrosine Kinase, Receptor, | 92 | Receptor tyrosine kinase |
| | | | Type 3 | | |
| 144 | ADAMTS-10 | Q9H324 | ADAM Metallopeptidase With | 120.9 | Metalloprotease that participate in |
| | | | Thrombospondin Type 1 Motif 10 | | microfibrils assembly. |
| 145 | Tarc | Q92583 | C-C motif chemokine 17 | 10.5 | Signalling chemokine. |
| | | | | | Chemotactic factor for T- |
| | | | | | lymphocytes but not monocytes or |
| | | | | | granulocytes. May play a role in T- |
| | | | | | cell development in thymus and in |
| | | | | | trafficking and activation of mature |
| | | | | | T-cells |
| 146 | MMP-10 | P09238 | Stromelysin-2 (matrix metallo proteinase 10) | 54.1 | Matrix metallo Proteinase |
| 147 | PARK7 | Q99497 | Protein deglycase DJ-1 | 19.9 | Protein deglycase that repairs |
| | | | | | methylglyoxal- and glyoxal- |
| | | | | | glycated amino acids and proteins |
| 148 | GDF-15 | Q99988 | Growth/differentiation factor 15 | 34.1 | belonging to the transforming |
| | | | | | growth factor beta superfamily that |
| | | | | | has a role in regulating |
| | | | | | inflammatory and apoptotic |
| | | | | | pathways in injured tissues and |
| | | | | | during disease processes |
| 149 | Vitronectin | P04004 | Vitronectin (VN) (S-protein) (Serum- | 54.3 | Glycoprotein promoting cell |
| | | | spreading factor) | | adhesion via integrins |
| 150 | IL-6 | P05231 | Interleukin-6 | 23.7 | Pro-inflammatory cytokine |
| 151 | HSPA8 | P11142 | Heat shock cognate 71 kDa protein | 70.9 | Acts as a repressor of transcriptional |
| | | | | | activation and a chaperone |
| 152 | Serpin A12 | Q8IW75 | Serpin A12 | 47.2 | Adipokine that modulates insulin |
| | | | | | action by specifically inhibiting its |
| | | | | | target protease KLK7 in white |
| | | | | | adipose tissues |
| 153 | IL-17RC | Q8NAC3 | Interleukin-17 receptor C | 86.2 | Pro-inflammatory cytokine |
| | | | | | receptor |
| 154 | PAI-1 | P05121 | Plasminogen activator inhibitor 1 | 45 | Serine protease inhibitor. This |
| | | | | | inhibitor acts as ‘bait’ for tissue |
| | | | | | plasminogen activator, urokinase |
| 155 | TCCR/WSX-1 | Q6UWB1 | Interleukin-27 receptor subunit alpha | 69.5 | Receptor for IL27. This signaling |
| | | | | | system acts through STAT3 and |
| | | | | | STAT1 and it is Involved in the |
| | | | | | regulation of Th1-type immune |
| | | | | | responses |
| 156 | BMP-5 | P22003 | Bone morphogenetic protein 5 | 51.7 | Induces cartilage and bone |
| | | | | | formation |
| 157 | Neurokinin-A | P20366 | Tachykinin Precursor 1 | 15 | Tachykinins are active peptides |
| | | | | | which excite neurons, evoke |
| | | | | | behavioral responses, are potent |
| | | | | | vasodilators and secretagogues, |
| | | | | | and contract (directly or indirectly) |
| | | | | | many smooth muscles |
| 158 | Beta IG-H3 | Q15582 | Transforming Growth Factor Beta | 74.7 | May play a role in cell-collagen |
| | | | Induced | | interactions |
| 159 | CNTF | P26441 | Ciliary neurotrophic factor | 22.9 | CNTF is a survival factor for |
| | | | | | various neuronal cell types. |
| 160 | Troponin C | P63316 | Troponin C, slow skeletal and cardiac | 18.4 | Central regulatory protein of |
| | | | muscles | | striated muscle contraction |
| 161 | Mammaglobin A | Q13296 | Mammaglobin-A | 10.5 | Expressed mainly in mucosa, |
| | | | | | secretoglobins seem to be |
| | | | | | involved in cell signalling, immune |
| | | | | | response, and chemotaxis, and |
| | | | | | may also serve as transporters for |
| | | | | | steroid hormones in humans |
| 162 | Serpin A1 | P01009 | Alpha-1-antitrypsin | 46.7 | Inhibitor of serine proteases |
| 163 | LRP-1 | Q07954 | Prolow-density lipoprotein receptor- | 504.6 | Endocytic receptor involved in |
| | | | related protein 1 | | endocytosis and in phagocytosis |
| | | | | | of apoptotic cells |
| 164 | L-Selectin | P14151 | L-selectin | 42.2 | Cell surface adhesion protein. |
| | (CD62L) | | | | Mediates the adherence of |
| | | | | | lymphocytes to endothelial cells |
| 165 | PYK2 | Q14289 | Protein-tyrosine kinase 2-beta | 115.9 | Non-receptor protein-tyrosine |
| | | | | | kinase that regulates |
| | | | | | reorganization of the actin |
| | | | | | cytoskeleton |
| 166 | TWEAK R/ | Q93038 | Tumor necrosis factor receptor | 45.4 | TNFR superfamily member |
| | TNFRSF12 | | superfamily member 25 | | |
| 167 | IL-26 | Q9NPH9 | Interleukin-26 | 19.8 | Pro-inflammatory Signalling |
| | | | | | cytokine |
| 168 | IL-15 R alpha | Q13261 | Interleukin-15 receptor subunit alpha | 28.2 | Cytokine receptor |
| 169 | VE-Cadherin | P33151 | Cadherin-5 | 87.5 | calcium-dependent cell adhesion |
| | | | | | proteins |
| 170 | TMEFF1/ | Q8IYR6 | Tomoregulin-1 | 40.9 | Transmembrane protein |
| | Tomoregulin-1 | | | | |
| 171 | Itk | Q08881 | Tyrosine-protein kinase ITK/TSK | 71.8 | Tyrosine kinase that plays an |
| | | | | | essential role in regulation of the |
| | | | | | adaptive immune response |
| 172 | S100A8 | P05109 | Protein S100-A8 | 10.8 | Calcium- and zinc-binding protein |
| | | | | | which plays a prominent role in |
| | | | | | the regulation of inflammatory |
| | | | | | processes and immune response |
| 173 | NRG3 | P56975 | Pro-neuregulin-3 | 77.9 | Direct activator of the ERBB4 |
| | | | | | tyrosine kinase receptor. |
| 174 | GFR alpha-4 | Q9GZZ7 | GDNF family receptor alpha-4 | 31.7 | Receptor for persephin |
| 175 | VEGF R2 | P35968 | Vascular endothelial growth factor | 151.5 | Tyrosine-protein kinase that acts |
| | (KDR) | | receptor 2 | | as a cell-surface receptor for |
| | | | | | VEGFA, VEGFC and VEGFD. |
| | | | | | Plays an essential role in the |
| | | | | | regulation of angiogenesis |
| 176 | Pro-MMP-13 | P45452 | Matrix Metallopeptidase 13 | 53.8 | Matrix metallo proteinase |
| | | | | | (collagenase 3) |
| 177 | IL-31 | Q6EBC2 | Interleukin-31 | 82.9 | Signalling cytokine |
| 178 | uPAR | Q03405 | Plasminogen Activator, Urokinase | 37 | Urokinase receptor |
| | | | Receptor | | |
| 179 | Livin | Q96CA5 | Baculoviral IAP repeat-containing protein 7 | 32.8 | Apoptotic regulator (mostly anti- |
| | | | | | apoptotic) |
| 180 | VCAM-1 | P19320 | Vascular cell adhesion protein 1 | 81.3 | Adhesion molecule |
| | (CD106) | | | | |
| 181 | D-Dimer | n/a | D dimer (a fibrin degradation product) | 200 | A fibrin degradation product |
| 182 | IL-17B R | Q9NRM6 | Interleukin-17 receptor B | 55.9 | Interleukin-17 receptor B |
| 183 | IL-17C | Q9P0M4 | Interleukin-17C | 21.7 | Interleukin 17 family member |
| 184 | IL-17D | Q8TAD2 | Interleukin-17D | 21.8 | Interleukin 17 family member |
| 185 | Hck | P08631 | HCK Proto-Oncogene, Src Family | 59.6 | Tyrosine-protein kinase HCK |
| | | | Tyrosine Kinase | | |
| 186 | Galanin | P22466 | Galanin peptides | 13.3 | Endocrine hormone |
| 187 | Lymphotactin/ | P47992 | Lymphotactin (ATAC) (C motif chemokine 1) | 12.5 | Pro-inflammatory Chemokine |
| | XCL1 | | | | |
| 188 | HTRA2 | O43464 | Serine protease HTRA2, mitochondrial | 48.8 | Serine protease (enzyme) |
| 189 | SSTR2 | P30874 | Somatostatin receptor type 2 | 41.3 | Receptor for somatostatin-14 and -28 |
| 190 | FRK | P42685 | Tyrosine-protein kinase FRK | 58.2 | Non-receptor tyrosine-protein |
| | | | | | kinase that negatively regulates |
| | | | | | cell proliferation |
| 191 | PTPRD | P23468 | Receptor-type tyrosine-protein | 214.8 | Receptor-type tyrosine-protein |
| | | | phosphatase delta | | phosphatase delta |
| 192 | MMP-20 | O60882 | Matrix metalloproteinase-20 | 54.4 | Matrix metallo proteinase |
| 193 | MIP-3 alpha | P78556 | C-C Motif Chemokine Ligand 20 | 13.4 | Chemokine for monocytes |
| 194 | Alpha 1 | P02760 | Alpha 1 Microglobulin | 27 | A1M binds and degrades heme, is |
| | Microglobulin | | | | a radical scavenger as well as |
| | | | | | a reductase |
| 195 | IL-31 RA | Q8NI17 | Interleukin-31 receptor subunit alpha | 82.9 | Interleukin-31 receptor subunit |
| | | | | | alpha |
| 196 | GPX3 | P22352 | Glutathione peroxidase 3 | 25.6 | Anti-oxidant enzyme |
| 197 | IBSP | P21815 | Bone sialoprotein 2 | 35.1 | Binds tightly to hydroxyapatite. |
| | | | | | Appears to form an integral part of |
| | | | | | the mineralized bone matrix. |
| | | | | | Probably important to cell-matrix |
| | | | | | interaction. |
| 198 | IL-1 R9 | Q9NP60 | X-linked interleukin-1 receptor | 78.7 | Member of the interleukin 1 |
| | | | accessory protein-like 2 | | receptor family |
| 199 | Endothelin | P05305 | Endothelin | 24.4 | It is a potent vasoconstrictor and |
| | | | | | is produced by |
| | | | | | vascular endothelial cells |
| 200 | pro-Glucagon | P01275 | Glucagon preprotein | 20.9 | Regulates blood glucose by |
| | | | | | increasing gluconeogenesis and |
| | | | | | decreasing glycolysis |
| 201 | GADD45A | P24522 | Growth Arrest And DNA Damage | 18.3 | It stimulates DNA excision repair |
| | | | Inducible Alpha | | and inhibits entry of cells into S |
| | | | | | phase |
| 202 | Omentin | Q8WWA0 | Omentin | 34.9 | It enhances insulin-stimulated |
| | | | | | glucose uptake in adipocytes |

The present disclosure has been described with reference to one or more exemplary data embodiments. However, it will be understood by those skilled in the art that various changes can be made, and equivalents can be substituted, without departing from the scope of the present disclosure. In addition, modifications can be made to adapt a particular material to the teachings of the present disclosure without departing from the scope thereof. Therefore, the present disclosure is not intended to be limited to the particular material or embodiment disclosed, but includes all materials and embodiments falling within the scope hereof.

What is claimed is:

1. A topical skin composition formulated to achieve a desired skin benefit, the composition comprising:
a blend of proteins that have been selected based on the desired skin benefit to be derived by application of the topical skin composition,
wherein each one of the blend of proteins are from a homogenous donor group having two or more individuals;
a delivery vehicle for the proteins,
wherein the blend of proteins is present in an amount from 0.0001 wt. % to 10 wt. % based on the total weight of the topical skin composition.

2. The topical skin composition of claim 1, wherein the amount is 0.001 wt. % to 5.0 wt. %.

3. The topical skin composition of claim 1, wherein the amount is 0.001 wt. % to 1.0 wt. %.

4. The topical skin composition of claim 1, wherein the homogenous donor group is selected from the group consisting of: Asian, African, and Caucasian.

5. The topical skin composition of claim 4, wherein the homogenous donor group comprises donors who constitute a group whose bloodlines are 80% or greater of a single race or ethnicity.

6. The topical skin composition of claim 1, wherein the proteins are synthetically derived.

7. The topical skin composition of claim 1, wherein the desired skin effect is at least one skin effect selected from the group consisting of: pigmentation, wound healing, inflammation, dermal matrix, wrinkles, scar prevention, and epidermis.

8. The topical skin composition of claim 1, wherein the topical skin composition is an emulsion.

9. The topical skin composition of claim 8, wherein the emulsion is an oil in water emulsion.

10. The topical skin composition of claim 1, wherein the vehicle includes at least one ingredient selected from the group consisting of one or more antioxidants, polypeptides, vitamins, plant extracts, materials derived from plant stem cells, oils, preservatives, thickening agents, ceramides, skin lighteners, exfoliants, anti-aging and anti-wrinkle agents, sunscreens, skin barrier repair agents, moisturizing ingredients, essential fatty acids, humectants, emollients, solvents, surfactants, emulsifiers, fillers, polymers, buffers, temperature regulating agents, and the like, and combinations thereof.

11. The topical skin composition of claim 10, wherein the vehicle can include water.

12. The topical skin composition of claim 11, wherein the vehicle is up to 99 wt. % based on the total weight of the topical skin composition.

13. A topical skin composition formulated to achieve a desired skin benefit, the composition comprising:
a blend of proteins that have been selected based on the desired skin benefit to be derived by application of the topical skin composition,
wherein the blend of proteins is from one or more donors from at least two or more donor groups;
a delivery vehicle for the proteins,
wherein the blend of proteins is present in an amount from 0.0001 wt. % to 10 wt. % based on the total weight of the topical skin composition.

14. The topical skin composition of claim 13, wherein the donor group is heterogenous and is selected from the group consisting of: Asian and African; Asian and Caucasian; African and Caucasian; and Asia, African, and Caucasian.

15. The topical skin composition of claim 13, wherein the amount is 0.001 wt. % to 5.0 wt. %.

16. The topical skin composition of claim 13, wherein the amount is 0.001 wt. % to 1.0 wt. %.

17. The topical skin composition of claim 13, wherein the proteins are synthetically derived.

18. The topical skin composition of claim 13, wherein the desired skin effect is at least one skin effect selected from the group consisting of: pigmentation, wound healing, inflammation, dermal matrix, wrinkles, scar prevention, and epidermis.

19. The topical skin composition of claim 13, wherein the topical skin composition is an emulsion.

20. The topical skin composition of claim 13, wherein the vehicle includes at least one ingredient selected from the group consisting of one or more antioxidants, polypeptides, vitamins, plant extracts, materials derived from plant stem cells, oils, preservatives, thickening agents, ceramides, skin lighteners, exfoliants, anti-aging and anti-wrinkle agents, sunscreens, skin barrier repair agents, moisturizing ingredients, essential fatty acids, humectants, emollients, solvents, surfactants, emulsifiers, fillers, polymers, buffers, temperature regulating agents, and the like, and combinations thereof.
